SEQUENCE LISTING (51 sequences)

SEQ ID NO: 1 - protein, 121 aa, Artificial Sequence
  Synthetic Sequence_a heavy chain (HC) variable region
QVQLVQSGAEVKKPGASVKVSCKASGNTFTSYDINWVRQATGQGLEWMGWMNPNSGNTGYAQKFQGRVTMTRNTSISTAYMELSSLRSEDTAVYYCVYYNYVWGSFRFDDWGQGTLVTVSS

SEQ ID NO: 2 - protein, 107 aa, Artificial Sequence
  Synthetic Sequence_a light chain (LC) variable region
DIQLTQSPSFLSASVGDRVTITCRASQGISSYLAWYQQKPGKAPKLLIYAASTLQSGVPSRFSGSGSGTEFTLAVSSLQPEDFATYYCQQLNSYPYTFGQGTKLEIK

SEQ ID NO: 3 - protein, 118 aa, Artificial Sequence
  Synthetic Sequence_a heavy chain (HC) variable region
QVQLVESGGGVVQPGRSLRLSCTASGFTFSHYGMHWVRQAPGKGLEWVSFLWFDGSKDNYADSVKGRFTISRDNSKNMLYLRMDSLRAEDTAVYYCARNGGLTIFDYWGQGTLVTVSS

SEQ ID NO: 4 - protein, 107 aa, Artificial Sequence
  Synthetic Sequence_a light chain (LC) variable region
AIQLTQSPSSLSASVGDRVTITCRASQGINSWLAWYQQKPEKAPKSLIYAASSLQSGVPSRFSGSGSGTDFTLTISSLQPEDFATYYCQQYNFYPYTFGQGTKLEII

SEQ ID NO: 5 - protein, 5 aa, Artificial Sequence
  Synthetic Sequence_a heavy chain variable region comprising CDR1
SYDIN

SEQ ID NO: 6 - protein, 17 aa, Artificial Sequence
  Synthetic Sequence_a heavy chain variable region comprising CDR2
WMNPNSGNTGYAQKFQG

SEQ ID NO: 7 - protein, 12 aa, Artificial Sequence
  Synthetic Sequence_a heavy chain variable region comprising CDR3
YNYVWGSFRFDD

SEQ ID NO: 8 - protein, 11 aa, Artificial Sequence
  Synthetic Sequence_a light chain variable region comprising CDR1
RASQGISSYLA

SEQ ID NO: 9 - protein, 7 aa, Artificial Sequence
  Synthetic Sequence_a light chain variable region comprising CDR2
AASTLQS

SEQ ID NO: 10 - protein, 9 aa, Artificial Sequence
  Synthetic Sequence_a light chain variable region comprising CDR3
QQLNSYPYT

SEQ ID NO: 11 - protein, 5 aa, Artificial Sequence
  Synthetic Sequence_a heavy chain variable region comprising CDR1
HYGMH

SEQ ID NO: 12 - protein, 17 aa, Artificial Sequence
  Synthetic Sequence_a heavy chain variable region comprising CDR2
FLWFDGSKDNYADSVKG

SEQ ID NO: 13 - protein, 9 aa, Artificial Sequence
  Synthetic Sequence_a heavy chain variable region comprising CDR3
NGGLTIFDY

SEQ ID NO: 14 - protein, 11 aa, Artificial Sequence
  Synthetic Sequence_a light chain variable region comprising CDR1
RASQGINSWLA

SEQ ID NO: 15 - protein, 7 aa, Artificial Sequence
  Synthetic Sequence_a light chain variable region comprising CDR2
AASSLQS

SEQ ID NO: 16 - protein, 9 aa, Artificial Sequence
  Synthetic Sequence
QQYNFYPYT

SEQ ID NO: 17 - protein, 220 aa, Homo sapiens
MASAGMQILGVVLTLLGWVNGLVSCALPMWKVTAFIGNSIVVAQVVWEGLWMSCVVQSTGQMQCKVYDSLLALPQDLQAARALCVIALLVALFGLLVYLAGAKCTTCVEEKDSKARLVLTSGIVFVISGVLTLIPVCWTAHAIIRDFYNPLVAEAQKRELGASLYLGWAASGLLLLGGGLLCCTCPSGGSQGPSHYMARYSTSAPAISRGPSEYPTKNYV

SEQ ID NO: 18 - protein, 220 aa, Macaca fascicularis
MASAGMQILGVVLTLLGWVNGLVSCALPMWKVTAFIGNSIVVAQVVWEGLWMSCVVQSTGQMQCKVYDSLLALPQDLQAARALCVIALLVALFGLLVYLAGAKCTTCVEEKDSKARLVLTSGIVFVISGVLTLIPVCWTAHAIIRDFYNPLVAEAQKRELGASLYLGWAASGLLLLGGGLLCCTCPSGGSRGPSHYMARYSTSAPAISRGPSEYPTKNYV

SEQ ID NO: 19 - protein, 219 aa, Mus musculus
MASTGLQILGIVLTLLGWVNALVSCALPMWKVTAFIGNSIVVAQMVWEGLWMSCVVQSTGQMQCKVYDSLLALPQDLQAARALCVVTLLIVLLGLLVYLAGAKCTTCVEDRNSKSRLVLISGIIFVISGVLTLIPVCWTAHSIIQDFYNPLVADAQKRELGASLYLGWAASGLLLLGGGLLCCACSSGGTQGPRHYMACYSTSVPHSRGPSEYPTKNYV

SEQ ID NO: 20 - protein, 217 aa, Homo sapiens
MASTGLELLGMTLAVLGWLGTLVSCALPLWKVTAFIGNSIVVAQVVWEGLWMSCVVQSTGQMQCKVYDSLLALPQDLQAARALCVIALLLALLGLLVAITGAQCTTCVEDEGAKARIVLTAGVILLLAGILVLIPVCWTAHAIIQDFYNPLVAEALKRELGASLYLGWAAAALLMLGGGLLCCTCPPPQVERPRGPRLGYSIPSRSGASGLDKRDYV

SEQ ID NO: 21 - protein, 209 aa, Homo sapiens
MASMGLQVMGIALAVLGWLAVMLCCALPMWRVTAFIGSNIVTSQTIWEGLWMNCVVQSTGQMQCKVYDSLLALPQDLQAARALVIISIIVAALGVLLSVVGGKCTNCLEDESAKAKTMIVAGVVFLLAGLMVIVPVSWTAHNIIQDFYNPLVASGQKREMGASLYVGWAASGLLLLGGGLLCCNCPPRTDKPYSAKYSAARSAAASNYV

SEQ ID NO: 22 - protein, 220 aa, Homo sapiens
MSMGLEITGTALAVLGWLGTIVCCALPMWRVSAFIGSNIITSQNIWEGLWMNCVVQSTGQMQCKVYDSLLALPQDLQAARALIVVAILLAAFGLLVALVGAQCTNCVQDDTAKAKITIVAGVLFLLAALLTLVPVSWSANTIIRDFYNPVVPEAQKREMGAGLYVGWAAAALQLLGGALLCCSCPPREKKYTATKVVYSAPRSTGPGASLGTGYDRKDYV

SEQ ID NO: 23 - protein, 121 aa, Artificial Sequence
  Synthetic Sequence_a variable heavy chain sequence
QVQLVQSGAEVKKPGASVKVSCKASGNTFTSYDINWVRQATGQGLEWMGWMNPNSGNTGYAQKFQGRVTMTRDTSISTAYMELSSLRSEDTAVYYCVYYNYVWGSFRFDDWGQGTLVTVSS

SEQ ID NO: 24 - protein, 124 aa, Artificial Sequence
  Synthetic Sequence_a variable heavy chain sequence
QVQLVESGGGVVQPGRSLRLSCAASGFTFSGYGMHWVRQAPGKGLEWVAAIWYDGSNKYYADSVKGRFTISSDNSKNTLYLQMKSLRAEDTAVYYCAREWYSSGWSMYDVFDIWGQGTMVTVSS

SEQ ID NO: 25 - protein, 110 aa, Artificial Sequence
  Synthetic Sequence_a variable light chain sequence
QTVVTQEPSFSVSPGGTVTLTCGLSSGSVSTSYYPSWYQQTPGQAPRTLIYSTNTRSSGVPDRFSGSILGNKAALTITGAQADDESDYYCVLYMGSGIWVFGGGTKLTVL

SEQ ID NO: 26 - protein, 128 aa, Artificial Sequence
  Synthetic Sequence_a variable heavy chain sequence
QVQLVESGGGVVQPGRSLRLSCAASGFIFSSYGMHWVRQAPGKGLEWVAVIWYDGSNKFYADSVKGRFTISRDNSRKTLYLQMNSLRAEDTAVYYCARDGRYFDWGGSYYYYYGMDVWGQGTTVTVSS

SEQ ID NO: 27 - protein, 107 aa, Artificial Sequence
  Synthetic Sequence_a variable light chain sequence
EIVLTQSPATLSLSPGERATLSCGASQSVSSSYLAWYQQKPGLAPRLLIYDTSSRATGIPDRFSGSGSGTDFTLTISRLEPEDFAVYYCQQYGSSYTFGQGTKLEIK

SEQ ID NO: 28 - protein, 128 aa, Artificial Sequence
  Synthetic Sequence_a variable heavy chain sequence
QVQLVESGGGAVQPGRSLRLSCAASGFIFSSYGMHWVRQAPGKGLEWVAIIWFDGRNKNYADSVKGRFTISRDNSKKTLYLEMSSLRAEDTAVYYCARDGRYFDWGGSYYYYYGMDVWGQGTTVTVSS

SEQ ID NO: 29 - protein, 107 aa, Artificial Sequence
  Synthetic Sequence_a variable light chain sequence
EIVLTQSPATLSLSPGERATLSCGASQSVSSSYLAWYQQKPGLAPRLLIYDASSRATGIPDRFSGSGSGTDFTLTISRLEPEDFAVYYCQQYGSSYTFGQGTKLEIK

SEQ ID NO: 30 - protein, 126 aa, Artificial Sequence
  Synthetic Sequence_a variable heavy chain sequence
QVQLQESGPGLVKPSETLSLTCTVSGGSISSYYWSWIRQPPGKGLEWIGYIYYSGSTNYNPSLKSRVTISLDTSKNQFSLKLTSVTAADTAVYYCARGSIPVAGTSYFYYYGMDVWGQGTTVTVSS

SEQ ID NO: 31 - protein, 108 aa, Artificial Sequence
  Synthetic Sequence_a variable light chain sequence
EIVMTQSPATLSLSPGERATLSCRASQSVSSSYLSWYQQKPGQAPRLLIYGASTRATGIPARFSGSGSGTDFTLTISSLQPEDFAVYYCQQDYNLPWTFGQGTKVEIK

SEQ ID NO: 32 - protein, 5 aa, Artificial Sequence
  Synthetic Sequence_a heavy chain variable region comprising CDR1
GYGMH

SEQ ID NO: 33 - protein, 17 aa, Artificial Sequence
  Synthetic Sequence_a heavy chain variable region comprising CDR2
AIWYDGSNKYYADSVKG

SEQ ID NO: 34 - protein, 15 aa, Artificial Sequence
  Synthetic Sequence_a heavy chain variable region comprising CDR3
EWYSSGWSMYDVFDI

SEQ ID NO: 35 - protein, 14 aa, Artificial Sequence
  Synthetic Sequence_a light chain variable region comprising CDR1
GLSSGSVSTSYYPS

SEQ ID NO: 36 - protein, 7 aa, Artificial Sequence
  Synthetic Sequence_a light chain variable region comprising CDR2
STNTRSS

SEQ ID NO: 37 - protein, 10 aa, Artificial Sequence
  Synthetic Sequence_a light chain variable region comprising CDR3
VLYMGSGIWV

SEQ ID NO: 38 - protein, 5 aa, Artificial Sequence
  Synthetic Sequence_a heavy chain variable region comprising CDR1
SYGMH

SEQ ID NO: 39 - protein, 17 aa, Artificial Sequence
  Synthetic Sequence_a heavy chain variable region comprising CDR2
VIWYDGSNKFYADSVKG

SEQ ID NO: 40 - protein, 19 aa, Artificial Sequence
  Synthetic Sequence_a heavy chain variable region comprising CDR3
DGRYFDWGGSYYYYYGMDV

SEQ ID NO: 41 - protein, 12 aa, Artificial Sequence
  Synthetic Sequence_a light chain variable region comprising CDR1
GASQSVSSSYLA

SEQ ID NO: 42 - protein, 7 aa, Artificial Sequence
  Synthetic Sequence_a light chain variable region comprising CDR2
DTSSRAT

SEQ ID NO: 43 - protein, 8 aa, Artificial Sequence
  Synthetic Sequence_a light chain variable region comprising CDR3
QQYGSSYT

SEQ ID NO: 44 - protein, 17 aa, Artificial Sequence
  Synthetic Sequence_a heavy chain variable region comprising CDR2
IIWFDGRNKNYADSVKG

SEQ ID NO: 45 - protein, 7 aa, Artificial Sequence
  Synthetic Sequence_a light chain variable region comprising CDR2
DASSRAT

SEQ ID NO: 46 - protein, 5 aa, Artificial Sequence
  Synthetic Sequence_a heavy chain variable region comprising CDR1
SYYWS

SEQ ID NO: 47 - protein, 16 aa, Artificial Sequence
  Synthetic Sequence_a heavy chain variable region comprising CDR2
YIYYSGSTNYNPSLKS

SEQ ID NO: 48 - protein, 18 aa, Artificial Sequence
  Synthetic Sequence_a heavy chain variable region comprising CDR3
GSIPVAGTSYFYYYGMDV

SEQ ID NO: 49 - protein, 12 aa, Artificial Sequence
  Synthetic Sequence_a light chain variable region comprising CDR1
RASQSVSSSYLS

SEQ ID NO: 50 - protein, 7 aa, Artificial Sequence
  Synthetic Sequence_a light chain variable region comprising CDR2
GASTRAT

SEQ ID NO: 51 - protein, 9 aa, Artificial Sequence
  Synthetic Sequence_a light chain variable region comprising CDR3
QQDYNLPWT

(19) United States
(12) Patent Application Publication
(10) Pub. No.: US 2025/0277026 A1
(43) Pub. Date: Sep. 4, 2025

(54) ANTIBODIES AGAINST CLAUDIN-6 AND USES THEREOF

(71) Applicant: NOVAROCK BIOTHERAPEUTICS, LTD., Ewing, NJ (US)

(72) Inventors: Haichun HUANG, Fremont, CA (US); Ming LEI, Princeton, NJ (US); Yi PEI, Paoli, PA (US); Han LI, Newton, PA (US); Huarui LU, Skillman, NJ (US)

(21) Appl. No.: 18/278,373

(22) PCT Filed: Mar. 1, 2022
(86) PCT No.: PCT/US22/18383

§ 371 Date: Aug. 22, 2023

Related U.S. Application Data
(60) Provisional application No. 63/240,399, filed on Sep. 3, 2021.
(60) Provisional application No. 63/155,304, filed on Mar. 2, 2021.

(51) Int. Cl. C07K 16/28; A61K 35/00; A61K 47/68; G01N 33/574

(52) U.S. Cl. CPC C07K 16/28; A61K 35/00; A61K 47/6849; G01N 33/574; C07K 2317/24; C07K 2317/565; C07K 2317/732; C07K 2317/77; C07K 2317/92; G01N 2333/705

(57) ABSTRACT

The present disclosure provides antibodies and antibody fragments thereof that bind to human CLDN6. The disclosed antibodies may be used in antibody-based immunotherapy methods to direct a cytotoxic response against, or to target CLDN6 expressing cancers for destruction by an antibody drug conjugates.

### CROSS REFERENCE TO RELATED APPLICATIONS

This PCT application claims priority to U.S. Application Ser. No. 63/240,399, entitled “ANTIBODIES AGAINST CLAUDIN-6 AND USES THEREOF,” filed on Sep. 3, 2021, and U.S. Application Ser. No. 63/155,304, entitled “ANTIBODIES AGAINST CLAUDIN-6 AND USES THEREOF,” filed on Mar. 2, 2021, the entirety of which are incorporated herein by reference.

### SEQUENCE LISTING

The instant application contains a Sequence Listing which has been submitted electronically in ASCII format and is hereby incorporated by reference in its entirety. Said ASCII copy, created on Mar. 11, 2024, is named “122863-5006-US_Sequence_Listing_ST25.txt” and is 42 kilobytes in size.

### FIELD

The present disclosure is in the field of immunotherapy and relates to antibodies and fragments thereof which bind to human Claudin-6 (CLDN6), to polynucleotide sequences encoding these antibodies and to cells producing them. The disclosure further relates to therapeutic compositions comprising these antibodies, and to methods of their use for cancer detection, prognosis and antibody-based immunotherapy.

### BACKGROUND

The Claudin (CLDN) family is composed of 27 members and displays distinct expression patterns in cell- and tissue-type-selective manners. Claudins are integral membrane proteins located within the tight junctions (TJs) of epithelia and endothelia. CLDNs interact with each other, both in the same cell (cis-interaction) and on adjacent cells (trans-interaction), resulting in the constitution of TJs with tissue-specific barrier functions. Individual cell types express more than one of the claudin family members. In normal physiology, the claudins interact with multiple proteins and are intimately involved in signal transduction to and from the tight junction (Lal-Nag, M and Morin, P. J., Genome Biol 10:235, 2009).

CLDN proteins comprise four transmembrane (TM) helices (TM1, TM2, TM3, and TM4) and two extracellular loops (ELI and EL2). The extracellular loops of claudins from adjacent cells interact with each other to seal the cellular sheet and regulate paracellular transport between the luminal and basolateral spaces. The claudin protein structure is highly conserved among the different family members and CLDN6 comprises 220 amino acids, is 23 kDa in size and exhibits a claudin-typical protein structure.

Unlike the majority of Claudin proteins that are broadly expressed, CLDN6 is characterized by selective expression (Hewitt, et al., BMC Cancer, 6:186, 2006). CLDN6 is an oncofetal tight junction molecule expressed in several types of embryonic epithelial cells. Disturbance of tight junctions and dysregulation of tight junction molecules is a frequent hallmark of cancer cells and frequently associated with malignant transformation. CLDN6 expression is aberrantly activated in various cancer types, including gastric, lung and ovarian adenocarcinomas, endometrial and embryonal carcinomas, pediatric tumors of the brain (e.g., atypical teratoid/rhabdoid tumors) and germ cell tumors (Hassimoto et al., J Pharmacol Exp Ther 368:179-186, 2019; Kojima et al., Cancers 2020, 12, 2748) Accordingly, CLDN6 has been identified as a tumor-associated antigen. As a tumor-associated antigen it can be classified as a differentiation antigen due to its expression during early stage of epidermal morphogenesis where it is crucial for epidermal differentiation and barrier formation. The distinct expression pattern of CLDN6 in cancer but not in adult normal tissues combined with its cell surface accessibility to antibodies qualifies CLDN6 as a promising target for diagnostic as well as immunotherapeutic approaches in a wide variety of cancer types.

There is a high degree of sequence conservation between CLDN6 to other claudin proteins. The high homology of CLDN6 with other Claudin proteins (e.g., CLDN9, CLDN4 and CLDN3) makes it difficult to provide CLDN6 antibodies which have properties suitable for therapeutic use such as specificity, affinity and safety.

Targeting Claudin-6 for antibody-based immunotherapy antibodies can modulate the activity of CLDN6 and/or direct a cytotoxic response against CLDN6 expressing cancer. Thus, there is a need for anti-CLDN6 antibodies.

### SUMMARY

The present disclosure addresses the above need by providing anti-CLDN6 antibodies and fragments that are capable of binding to Claudin-6 present on cancer cells. These antibodies and fragments thereof are characterized by unique sets of CDR sequences, specificity for CLDN6 and are useful in cancer detection, prognosis and immunotherapy. More specifically, the disclosure relates to antibodies that bind to human CLDN6, and to their use to detect the presence of CLDN-6 expressing tumor cells and/or tumor stem cells, to modulate a CLDN6-mediated activity of cells localized to the tumor microenvironment or to direct a cytotoxic response against CLDN6 expressing cancer.

According to some embodiments, the antibody or antibody fragments comprise a set of six complementarity determining region (CDRs) sequences selected from the group consisting of three CDRs of a heavy chain (HC) variable region selected from SEQ ID NOs: 1, 3, 23, 24, 26, 28 and 30, and three light CDRs of a light chain (LC) variable region selected from SEQ ID NOs: 2, 4, 25, 27, 29 and 31 or an analog or derivative thereof having at least 90% sequence identity with the identified antibody or fragment sequence.

In some embodiments, the anti-CLDN6 antibodies or antibody fragments thereof comprise a heavy chain variable region comprising CDR1: SEQ ID NO: 5, CDR2: SEQ ID NO: 6 and CDR3: SEQ ID NO: 7; and/or a light chain variable region comprising CDR1: SEQ ID NO: 8, CDR2: SEQ ID NO: 9, and CDR3: SEQ ID NO: 10.

In some embodiments, the anti-CLDN6 antibodies or antibody fragments thereof comprise a heavy chain variable region comprising CDR1: SEQ ID NO: 11, CDR2: SEQ ID NO: 12, and CDR3: SEQ ID NO: 13; and/or a light chain variable region comprising CDR1: SEQ ID NO: 14, CDR2: SEQ ID NO: 15, and CDR3: SEQ ID NO: 16.

In some embodiments, the anti-CLDN6 antibodies or antibody fragments thereof comprise a heavy chain variable region comprising CDR1: SEQ ID NO: 32, CDR2: SEQ ID NO: 33 and CDR3: SEQ ID NO: 34; and/or a light chain variable region comprising CDR1: SEQ ID NO: 35, CDR2: SEQ ID NO: 36, and CDR3: SEQ ID NO: 37.

In some embodiments, the anti-CLDN6 antibodies or antibody fragments thereof comprise a heavy chain variable region comprising CDR1: SEQ ID NO: 38, CDR2: SEQ ID NO: 39 and CDR3: SEQ ID NO: 40; and/or a light chain variable region comprising CDR1: SEQ ID NO: 41, CDR2: SEQ ID NO: 42, and CDR3: SEQ ID NO: 43.

In some embodiments, the anti-CLDN6 antibodies or antibody fragments thereof comprise a heavy chain variable region comprising CDR1: SEQ ID NO: 38, CDR2: SEQ ID NO: 44 and CDR3: SEQ ID NO: 40; and/or a light chain variable region comprising CDR1: SEQ ID NO: 41, CDR2: SEQ ID NO: 45, and CDR3: SEQ ID NO: 43.

In some embodiments, the anti-CLDN6 antibodies or antibody fragments thereof comprise a heavy chain variable region comprising CDR1: SEQ ID NO: 46, CDR2: SEQ ID NO: 47 and CDR3: SEQ ID NO: 48; and/or a light chain variable region comprising CDR1: SEQ ID NO: 49, CDR2: SEQ ID NO: 50, and CDR3: SEQ ID NO: 51.

In some embodiments, the anti-CLDN6 antibodies or antibody fragments thereof comprise a variable heavy chain sequence selected from the group consisting of SEQ ID NOs: 1, 3, 23, 24, 26, 28 and 30.

In other embodiments, the anti-CLDN6 antibodies or antibody fragments thereof comprise a variable light chain sequence selected from the group consisting of SEQ ID NOs: 2, 4, 25, 27, 29 and 31.

In other embodiments, the anti-CLDN6 antibodies or antibody fragments thereof comprise a variable heavy chain sequence selected from the group consisting of SEQ ID NOs: 1, 3, 23, 24, 26, 28 and 30 and a variable light chain sequence selected from the group consisting of SEQ ID NOs: 2, 4, 25, 27, 29 and 31.

In some embodiments, the anti-CLDN6 antibodies or antibody fragment comprises a variable heavy chain and variable light chain sequence, selected from the following combinations:
(a) a variable heavy chain sequence comprising SEQ ID NO: 1 and a variable light chain sequence comprising SEQ ID NO: 2;
(b) a variable heavy chain sequence comprising SEQ ID NO: 3 and a variable light chain sequence comprising SEQ ID NO: 4
(c) a variable heavy chain sequence comprising SEQ ID NO: 23 and a variable light chain sequence comprising SEQ ID NO: 2;
(d) a variable heavy chain sequence comprising SEQ ID NO: 24 and a variable light chain sequence comprising SEQ ID NO: 25;
(e) a variable heavy chain sequence comprising SEQ ID NO: 26 and a variable light chain sequence comprising SEQ ID NO: 27;
(f) a variable heavy chain sequence comprising SEQ ID NO: 28 and a variable light chain sequence comprising SEQ ID NO: 29; and
(g) a variable heavy chain sequence comprising SEQ ID NO: 30 and a variable light chain sequence comprising SEQ ID NO: 31.

In some embodiments, an immunoconjugate comprising an antibody that binds CLDN6 covalently attached to a cytotoxic agent is provided, wherein the antibody comprises a variable heavy chain and a variable light chain sequences, selected from the following combinations:
(a) a variable heavy chain sequence comprising SEQ ID NO: 1 and a variable light chain sequence comprising SEQ ID NO: 2; and
(b) a variable heavy chain sequence comprising SEQ ID NO: 3 and a variable light chain sequence comprising SEQ ID NO: 4
(c) a variable heavy chain sequence comprising SEQ ID NO: 23 and a variable light chain sequence comprising SEQ ID NO: 2;
(d) a variable heavy chain sequence comprising SEQ ID NO: 24 and a variable light chain sequence comprising SEQ ID NO: 25;
(e) a variable heavy chain sequence comprising SEQ ID NO: 26 and a variable light chain sequence comprising SEQ ID NO: 27;
(f) a variable heavy chain sequence comprising SEQ ID NO: 28 and a variable light chain sequence comprising SEQ ID NO: 29; and
(g) a variable heavy chain sequence comprising SEQ ID NO: 30 and a variable light chain sequence comprising SEQ ID NO: 31.

In some embodiments, an immunoconjugate comprising an antibody that binds Claudin-6 covalently attached to a cytotoxic agent is provided, wherein the antibody comprises (a) a heavy chain variable region comprising CDR1: SEQ ID NO: 5, CDR2: SEQ ID NO: 6, and CDR3: SEQ ID NO: 7; and/or a light chain variable region comprising CDR1: SEQ ID NO: 8, CDR2: SEQ ID NO: 9, and CDR3: SEQ ID NO: 10; (b) a heavy chain variable region comprising CDR1: SEQ ID NO: 11, CDR2: SEQ ID NO: 12, and CDR3: SEQ ID NO: 13; and/or a light chain variable region comprising CDR1: SEQ ID NO: 14, CDR2: SEQ ID NO: 15, and CDR3: SEQ ID NO: 16; (c) a heavy chain variable region comprising CDR1: SEQ ID NO: 32, CDR2: SEQ ID NO: 33, and CDR3: SEQ ID NO: 34; and/or a light chain variable region comprising CDR1: SEQ ID NO: 35, CDR2: SEQ ID NO: 36, and CDR3: SEQ ID NO: 37; (d) a heavy chain variable region comprising CDR1: SEQ ID NO: 38, CDR2: SEQ ID NO: 39, and CDR3: SEQ ID NO: 40; and/or a light chain variable region comprising CDR1: SEQ ID NO: 41, CDR2: SEQ ID NO: 42, and CDR3: SEQ ID NO: 43; (e) a heavy chain variable region comprising CDR1: SEQ ID NO: 38, CDR2: SEQ ID NO: 44, and CDR3: SEQ ID NO: 40; and/or a light chain variable region comprising CDR1: SEQ ID NO: 41, CDR2: SEQ ID NO: 45, and CDR3: SEQ ID NO: 43; and (f) a heavy chain variable region comprising CDR1: SEQ ID NO: 46, CDR2: SEQ ID NO: 47, and CDR3: SEQ ID NO: 48; and/or a light chain variable region comprising CDR1: SEQ ID NO: 49, CDR2: SEQ ID NO: 50, and CDR3: SEQ ID NO: 51.

In some embodiments, the anti-CLDN6 antibodies and antibody fragments thereof comprise one or more heavy chain variable region CDRs disclosed in Table 1 and/or one or more light chain variable region CDRs disclosed in Table 2.

In some embodiments, the anti-CLDN6 antibodies or antibody fragments thereof exhibit one or more of the following structural and functional characteristics, alone or in combination: (a) bind to cells expressing human CLDN6 on their cell surface; (b) selectively bind to Claudin-6 CHO-K1 cells with a signal (e.g., MFI) that is approximately 20 to 25-fold greater than the binding activity of an isotype control antibody for CLDN6 expressed on Claudin-6-CHO-K1 cells, or 20 to 30-fold greater than the binding activity of an isotype control antibody for CLDN6 expressed on Claudin-6-HEK293 cell and to Claudin-9 HEK293 cells with a signal that is only 16-fold greater than the binding activity of an isotype control antibody; (c) bind to Claudin-6 (Claudin-6 CHO-K1 cells) and Claudin-9 (HEK293 cells) expressing cells equally with a signal that it at approximately 25 to 60 fold greater than the binding activity of an isotype control antibody; (d) binds weakly or not at all to cells expressing CLDN3, CLDN4; (e) bind to NEC8 cells endogenously expressing Claudin-6, but do not bind to NEC8 cells with a knockout of the Claudin 6 gene; (f) optionally cross-react with murine CLDN6; (g) are efficiently internalized from the surface of Claudin-6 positive cells after binding and induce endocytosis-mediated cell cytotoxicity in NEC8 cells endogenously expressing Claudin-6; and (h) exhibit one or more immune effector functions against a cell carrying CLDN6 in its native conformation, wherein the one or more immune effector functions is selected antibody-dependent cell-mediated cytotoxicity (ADCC), T cell dependent cellular cytotoxicity (TDCC), complement dependent cytotoxicity (CDC), or antibody-dependent cellular phagocytosis (ADCP).

In some embodiments, the anti-CLDN6 antibodies specifically bind to human cells expressing endogenous levels of Claudin-6 and to host cells engineered to overexpress Claudin-6, and do not demonstrate binding to Claudin-3 or Claudin-4. In one embodiment, the anti-CLDN6 antibodies bind endogenously expressed CLDN6 on human testicular embryonal carcinoma (NEC8 cells). In some embodiments, the anti-CLDN6 antibodies bind endogenously expressed CLDN6 on human ovarian carcinoma (OV90 cells). In some embodiments, the CLDN6-specific antibodies or antibody fragments bind human Claudin-6 with an affinity below 100 nM. In other embodiments, the CLDN6/9-specific antibodies or antibody fragments selectively bind to human CLDN6 and human CLDN9.

In some embodiments the antibodies are capable of binding to CLDN6 associated with the surface of a cell that expresses CLDN6. Preferably, the CLDN6-expressing cell is an intact cell, in particular a non-permeabilized cell, and the CLDN6 protein bound by the antibodies is associated with the surface of a cell has a native, e.g., non-denatured, conformation. In a particular embodiment, the antibodies are not substantially capable of: binding to CLDN9 associated with the surface of a cell that expresses CLDN9; or binding to CLDN4 associated with the surface of a cell that expresses CLDN4 and/or binding to CLDN3 associated with the surface of a cell that expresses CLDN3.

In one embodiment, the anti-CLDN6 antibodies or antibody fragments selectively bind to CLDN6 relative to CLDN9 and do not bind to Claudin-3 (CLDN3), Claudin-4 (CLDN4). In another embodiment, the anti-CLDN6 antibodies or antibody fragments bind to both CLDN6 and CLDN 9 equally (e.g., no preference for either CLDN). In some embodiments the antibodies exhibit an EC₅₀ of less than about 50 nM (e.g., less than about 75 nM, less than about 50 nM, less than about 25 nM, less than about 10 nM) in a FACS-based assay using either host cells engineered to over express CLDN6 (e.g., 293T-CLDN6 or CHO-CLDN6).

In some embodiments, the antibody or fragment thereof exhibits one or more immune effector functions against a cell carrying CLDN6 in its native conformation, wherein the one or more immune effector functions are preferably selected from the group consisting of antibody-dependent cell-mediated cytotoxicity (ADCC), complement dependent cytotoxicity (CDC), induction of apoptosis, and inhibition of proliferation, preferably the effector functions are ADCC and/or CDC.

In some embodiments the antibody or fragment thereof may be attached to one or more therapeutic effector moieties, e.g., radiolabels, cytotoxins, therapeutic enzymes, agents that induce apoptosis, and the like in order to provide for targeted cytotoxicity, e.g., killing of tumor cells. In one embodiment, an anti-CLDN6 antibody specifically binds human CLDN6 as it occurs on the surface of tumor cells and efficiently induces the internalization of CLDN6 or directs cell-mediated killing of the tumor cell.

In some embodiments, an anti-CLDN6 antibody is incorporated into an immunoconjugate comprising an anti-CLDN6 antibody conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes (e.g., a radioconjugate).

In some embodiments, the anti-CLDN6 antibodies or antibody fragments may comprise substitutions or modifications of the constant region (i.e. the Fc region), including without limitation, amino acid residue substitutions, mutations and/or modifications, which result in a compound with one or more enhanced ADCC, CDC, ADCP, TDCC antibody-mediated effector functions.

In some embodiments, the anti-CLDN6 antibodies or antibody fragments comprises six (6) CDRs or variants thereof, derived from the VH or VL domain of a single antiCLDN6 antibody disclosed herein. For example, a binding agent may comprise all six of the CDR regions of the anti-CLDN6 antibody designated “NR.N6.Ab1.” In a representative example, an antibody or antibody fragment thereof may comprise the amino acid sequences of SEQ ID NOs: 5-7 and SEQ ID NOs: 8-10, representing the CDR1, CDR2 and CDR3 of the variable heavy chain region and the CDR1, CDR2 and CDR3 of the variable light chain region of the anti-human CLDN6 antibody referred to herein as “NR.N6.Ab1.”

Any of the anti-CLDN6 antibodies disclosed herein may be a fully human, chimeric, CDR grafted, humanized, or recombinant antibody, or a fragment thereof. In alternative embodiments, the disclosed anti-CLDN6 antibodies or antibody fragments may be developed for use in various alternative formats, including a bispecific or multi-specific format.

In some embodiments, the CLDN6 antibody is a full-length antibody. In some embodiments, the antibody is an antibody fragment. In further embodiments, the antibody fragment is selected from the group consisting of: Fab, Fab′, F(ab′)₂, Fd, Fv, scFv and scFv-Fc fragment, a single-chain antibody, a minibody, and a diabody.

The disclosure also provides a nucleic acid encoding any of the anti-CLDN6 antibodies disclosed herein. In a related embodiment, the disclosure provides a vector comprising one or more of the nucleic acids encoding an anti-CLDN6 antibody disclosed herein or a host cell comprising said vector.

The CLDN6 antibodies and antibody fragments thereof may be used for the treatment of cancer. Cancer cells expressing CLDN6 are suitable targets for therapies targeting CLDN6 such as therapy with antibodies directed against CLDN6. Such methods for the treatment or cancer may comprise administering a composition or formulation that comprises an CLDN6 antibody or antibody fragment thereof to a subject in need thereof.

For example, the CLDN6 antibody or antibody fragment thereof may be administered either alone (e.g., as a monotherapy) or in combination with other immunotherapeutic agent and/or a chemotherapy. In an embodiment, the CLDN6 antibody or fragment is used to prepare an ADC suitable to mediate the killing of cancer cells expressing CLDN6. In an alternative embodiment, the CLDN6 antibody is used to engineer a recombinant antibody designed to kill tumor cells by enhanced ADCC, ADCP, TDCC, or CDD.

The disclosure also provides a method of treating cancer comprising administering to a subject in need thereof a pharmaceutical composition comprising an ADC, wherein the ADC comprises an anti-CLDN6 antibody or antibody fragment disclosed herein conjugated to a payload. In some embodiments, the methods comprise administering to a subject in need thereof a pharmaceutical composition comprising an anti-CLDN ADC, wherein the cancer is selected from uterine, testicular, ovarian and lung cancer.

### BRIEF DESCRIPTION OF THE OF THE DRAWINGS

The foregoing summary, as well as the following detailed description of the disclosure, will be better understood when read in conjunction with the appended figures. For the purpose of illustrating the disclosure, shown in the figures are embodiments which are presently preferred. It should be understood, however, that the disclosure is not limited to the precise arrangements, examples and instrumentalities shown.

FIG. 1 provides the amino acid sequences of the VH and VL domains of the human anti-Claudin-6 antibodies and their respective CDR sequences (Kabat numbering). Sequence identifiers are provided and the CDRs are underlined in the variable domain sequences.

FIGS. 2A and 2B show binding of anti-CLDN6 antibodies and an isotype control antibody to Claudin-6-CHO-K1 transfected cells and non-transfected parental CHO-K1 cells by FACS.

FIGS. 3A and 3B show binding of anti-CLDN6 antibodies and an isotype control antibody to Claudin-9-HEK293 transfected cells by FACS.

FIGS. 4A and 4B show binding of anti-Claudin antibodies to Claudin-3-CHO-K1 transfected cells by FACS.

FIGS. 5A and 5B show binding of anti-Claudin antibodies to Claudin-4-CHO-K1 transfected cells by FACS.

FIGS. 6A and 6B show binding of anti-CLDN6 antibodies to NEC8 tumor cells endogenously expressing human CLDN6 by FACS.

FIGS. 7A and 7B show binding of anti-CDN6 antibodies to OV90 tumor cells endogenously expressing human CLDN6 by FACS.

FIGS. 8A and 8B show binding of anti-CLDN antibodies to MCF7 cells endogenously expressing Claudin-3 (CLDN3) and Claudin-4 (CLDN4) by FACS.

FIG. 9 shows binding of a rabbit polyclonal anti-CLDN9 antibody to the human tumor cell lines, NEC8, OV90 and MCF7 and a Claudin-9-HEK293 transfected cell by FACS.

FIGS. 10A, 10B and 10C show dose response curves for the disclosed anti-Claudin-6 antibodies, NR.N6.Ab1 and NR.N6.Ab2, binding to Claudin-6 overexpressing cell lines by FACS. HEK293 cells overexpressing Claudin-6 (10A); HEK293 cells overexpressing Claudin-9 (10B); and CHO cells overexpressing Claudin-6 (10C).

FIGS. 11A and 11B demonstrates NR.N6.Ab1 and NR.N6.Ab2-mediated antibody-dependent cellular cytotoxicity (ADCC) on NEC8 cells (FIG. 11A) and OV90 cells (FIG. 11B) endogenously expressing human CLDN6.

FIGS. 12A, 12B and 12C demonstrate antibody-mediated endocytosis induced by anti-Claudin-6 antibodies NR.N6.Ab1 and NR.N6.Ab2 on NEC8 tumor cells (FIG. 12A); OV90 tumor cells (FIG. 12B) endogenously expressing human CLDN6 and on Claudin-6 overexpressing cell line HEK293 cell (FIG. 12C).

FIGS. 13A, 13B, 13C and 13D show binding of anti-CLDN6 antibodies, NR.N6.Ab3, NR.N6.Ab4, NR.N6.Ab5, NR.N6.Ab6, and an isotype control antibody to HEK293 cells overexpressing Claudin-6 and HEK293 cells overexpressing Claudin-9 by FACS.

FIGS. 14A, 14B, 14B, 14C, 14D and 14E show binding of anti-CLDN6 antibodies, NR.N6.Ab3, NR.N6.Ab4, NR.4.N6.Ab5, NR.N6.Ab6, NR.NR6.Ab1 and an isotype control antibody to NEC8 endogenously expressing Claudin-6 and NEC8 Cluadin-6 gene knock out cells (NEC8 Claudin-6 KO) by FACS.

FIGS. 15A, 15B, 15C, 15D, 15E and 15F show binding of anti-CLDN6 antibodies, NR.N6.Ab3, NR.N6.Ab4, NR.N6.Ab5 and NR.N6.Ab6, and two positive controls MAB4620 and MAB4219 to CHO-K1 cells overexpressing Claudin-6, CHO-K1 cells overexpressing Claudin-3 and CHO-K1 cells overexpressing Claudin 4 by FACS.

FIGS. 16A, 16B, 16C and 16D show dose response curves for the disclosed anti-CLDN6 antibodies, NR.N6.Ab3, NR.N6.Ab4, NR.N6.Ab5 and NR.N6.Ab6, to HEK293 cells overexpressing Claudin-6 and HEK293 cells overexpressing Claudin-9 by FACS.

FIGS. 17A, 17B, 17C and 17D show dose response curves for the disclosed anti-CLDN6 antibodies, NR.N6.Ab3, NR.N6.Ab4, NR.N6.Ab5 and NR.N6.Ab6, to NEC8 endogenously expressing Claudin-6 and NEC8 Cluadin-6 gene knock out (NEC8 Claudin-6 KO) cells by FACS.

FIGS. 18A, 18B, 18C and 18D show dose response curves for the disclosed anti-CLDN6 antibodies, NR.N6.Ab1, variant of NR.N6.Ab1N73D and an isotype control, to HEK293 cells overexpressing Claudin-6, HEK293 cells overexpressing Claudin-9, NEC8 cells endogenously expressing Claudin-6 and NEC8 Claudin-6 KO cells by FACS.

FIGS. 19A and 19B demonstrates NR.N6.Ab1, NR.N6.Ab3, NR.N6.Ab4 and NR.N6.Ab5 mediated antibody-dependent cellular cytotoxicity (ADCC) on NEC8 endogenously expressing human CLDN6 cells (FIG. 19A) and NEC8 Claudin-6 knock out cells (FIG. 19B)

FIGS. 20A and 20B showed internalization activity of NR.N6.PC1, NR.N6.Ab1, NR.N6.Ab3, NR.N6.Ab4, NR.N6.Ab5 on NEC8 and NEC8 Claudin-6 knock out cells.

FIGS. 21A and 21B demonstrate antibody-mediated endocytosis induced by anti-Claudin-6 antibodies NR.N6.Ab1, NR.N6.Ab3, NR.N6.Ab4 and NR.N6.Ab5 on NEC8 tumor cells (FIG. 21A) endogenously expressing human CLDN6 and on NEC8 Claudin-6 knock out cells (FIG. 21B).

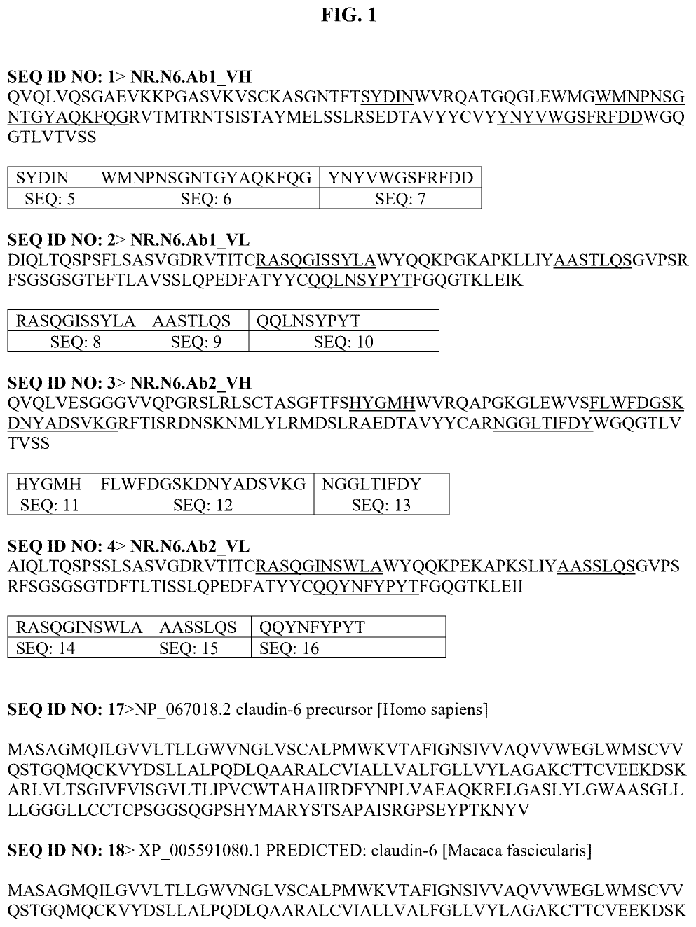

### DETAILED DESCRIPTION

So that the disclosure may be more readily understood, certain technical and scientific terms are specifically defined below. Unless specifically defined elsewhere in this document, all other technical and scientific terms used herein have the meaning commonly understood by one of ordinary skill in the art to which this disclosure belongs.

Throughout this disclosure the following abbreviations will be used:
mAb or Mab or MAb—Monoclonal antibody.
CDR—Complementarity determining region in the immunoglobulin variable regions.
VH or VH—Immunoglobulin heavy chain variable region.
VL or VL—Immunoglobulin light chain variable region.
FR—Antibody framework region, the immunoglobulin variable regions excluding the CDR regions

The terms “Claudin-6” or “CLDN6” (used interchangeably herein) preferably relates to human CLDN6, and, in particular, to a protein comprising the amino acid sequence according to SEQ ID NO: 1 of the sequence listing or a variant of said amino acid sequence. The term “CLDN6” includes any CLDN6 variants such as post-translationally modified variants and conformation variants. The amino acid sequences for human, cynomolgus, and murine CLDN6 are provided in NCBI Reference Sequences: NP_067018.2 (human) (SEQ ID NO: 17), XP_005591080.1 (cynomolgus monkey (SEQ ID NO: 18), and NP_061247.1 (mouse) (SEQ ID NO: 19). Orthologs of CLDN6 share >99% and ˜88% identity to the human protein in cynomolgus monkey and mice, respectively.

The term “CLDN9” relates to human CLDN9, and, in particular, to a protein comprising the amino acid sequence according to SEQ ID NO: 20 (NCBI Reference Sequence NP_066192.1) or a variant of said amino acid sequence. Human CLDN6 and human CLDN9 proteins share 71.8% identity.

The term “CLDN4” relates to human CLDN4, and, in particular, to a protein comprising the amino acid sequence according to SEQ ID NO: 21 (NCBI Reference Sequence NP_001296.1) or a variant of said amino acid sequence. Human CLDN6 and human CLDN4 proteins share 59.1% identity.

The term “CLDN3” relates to human CLDN3, and, in particular, to a protein comprising the amino acid sequence according to SEQ ID NO: 22 (NCBI Reference Sequence NP_001297.1) or a variant of said amino acid sequence. Human CLDN6 and human CLDN3 proteins share 56.7% identity.

The term “percentage identity” is intended to denote a percentage of amino acid residues which are identical between the two sequences to be compared, obtained after the best alignment, this percentage being purely statistical and the differences between the two sequences being distributed randomly and over their entire length. Sequence comparisons between two amino acid sequences are conventionally carried out by comparing these sequences after having aligned them optimally, said comparison being carried out by segment or by “window of comparison” in order to identify and compare local regions of sequence similarity. The optimal alignment of the sequences for comparison may be produced, besides manually, by means of the local homology algorithm of Smith and Waterman: 1981, Ads App. Math. 2, 482, by means of the local homology algorithm of Neddiernan and Wunsch, 1970, J. Mol. Biol. 48, 443, by means of the similarity search method of Pearson and Lipman, 1988, Proc. Natl Acad. Sci. USA 85, 2444, or by means of computer programs which use these algorithms (GAP, BESTFIT, FASTA, BLAST P, BLAST N and TFASTA in Wisconsin Genetics Software Package, Genetics Computer Group, 575 Science Drive, Madison, Wis.).

The term “antibody” herein is used in the broadest sense and encompasses various antibody structures, including but not limited to monoclonal antibodies, polyclonal antibodies, and multispecific antibodies (e.g., bispecific antibodies).

The term “cross-reacts,” as used herein, refers to the ability of anti-human CLDN6 antibodies described herein to bind to CLDN6 from a different species. For example, an antibody described herein may also bind CLDN6 from another species (e.g., or rat, or mouse CLDN6).

An exemplary antibody such as an IgG comprises two heavy chains and two light chains. Each heavy chain is comprised of a heavy chain variable region (abbreviated herein as VH) and a heavy chain constant region. Each light chain is comprised of a light chain variable region (abbreviated herein as VL) and a light chain constant region. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino terminus to carboxy-terminus in the following order: FR1, CDR1, FR2, CDR2, FR3, CDR3, FR4.

The hypervariable region generally encompasses amino acid residues from about amino acid residues 24-34 (LCDR1; “L” denotes light chain), 50-56 (LCDR2) and 89-97 (LCDR3) in the light chain variable region and around about 31-35B (HCDR1; “H” denotes heavy chain), 50-65 (HCDR2), and 95-102 (HCDR3) in the heavy chain variable region; Kabat et al., SEQUENCES OF PROTEINS OF IMMUNOLOGICAL INTEREST, 5th Ed. Public Health Service, National Institutes of Health, Bethesda, Md. (1991) and/or those residues forming a hypervariable loop (e.g. residues 26-32 (LCDR1), 50-52 (LCDR2) and 91-96 (LCDR3) in the light chain variable region and 26-32 (HCDR1), 53-55 (HCDR2) and 96-101 (HCDR3) in the heavy chain variable region; Chothia and Lesk (1987) J. Mol. Biol. 196:901-917.

The term “monoclonal antibody” as used herein refers to an antibody obtained from a population of substantially homogeneous antibodies, e.g., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies and is not to be construed as requiring production of the antibody by any method. For example, the monoclonal antibodies to be used in accordance with the present disclosure may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “chimeric” antibody refers to a recombinant antibody in which a portion of the heavy and/or light chain is identical with or homologous to corresponding sequences in antibodies derived from a particular species, or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity. In addition, complementarity determining region (CDR) grafting may be performed to alter certain properties of the antibody molecule including affinity or specificity. Typically, the variable domains are obtained from an antibody from an experimental animal (the “parental antibody”), such as a rodent, and the constant domain sequences are obtained from human antibodies, so that the resulting chimeric antibody can direct effector functions in a human subject and will be less likely to elicit an adverse immune response than the parental {e.g., mouse) antibody from which it is derived.

The term “humanized antibody” refers to an antibody that has been engineered to comprise one or more human framework regions in the variable region together with non-human (e.g., mouse, rat, or hamster) complementarity-determining regions (CDRs) of the heavy and/or light chain. In certain embodiments, a humanized antibody comprises sequences that are entirely human except for the CDR regions. Humanized antibodies are typically less immunogenic to humans, relative to non-humanized antibodies, and thus offer therapeutic benefits in certain situations. Those skilled in the art will be aware of humanized antibodies and will also be aware of suitable techniques for their generation. See for example, Hwang, W. Y. K., et al., Methods 36:35, 2005; Queen et al., Proc. Natl. Acad. Sci. USA, 86:10029-10033, 1989; Jones et al., Nature, 321:522-25, 1986; Riechmann et al., Nature, 332:323-27, 1988; Verhoeyen et al., Science, 239:1534-36, 1988; Orlandi et al., Proc. Natl. Acad. Sci. USA, 86:3833-37, 1989; U.S. Pat. Nos. 5,225,539; 5,530,101; 5,585,089; 5,693,761; 5,693,762; 6,180,370; and Selick et al., WO 90/07861, each of which is incorporated herein by reference in its entirety.

A “human antibody” is an antibody that possesses an amino-acid sequence corresponding to that of an antibody produced by a human and/or has been made using any of the techniques for making human antibodies known to one of skill in the art. This definition of a human antibody specifically excludes a humanized antibody comprising non-human antigen-binding residues. Human antibodies can be produced using various techniques known in the art, including methods described in Cole et al, Monoclonal Antibodies and Cancer Therapy, Alan R. Liss, p. 77 (1985); Boerner et al, J. Immunol, 147 (I): 86-95 (1991). See also van Dijk and van de Winkel, Curr. Opin. Pharmacol, 5:368-74 (2001). Human antibodies can be prepared by administering the antigen to a transgenic animal that has been modified to produce such antibodies in response to antigenic challenge, but whose endogenous loci have been disabled, e.g., immunized HuMab mice (see, e.g., Nils Lonberg et al., 1994, Nature 368:856-859, WO 98/24884, WO 94/25585, WO 93/1227, WO 92/22645, WO 92/03918 and WO 01/09187 regarding HuMab mice), xenomice (see, e.g., U.S. Pat. Nos. 6,075,181 and 6,150,584 regarding XENOMOUSE™ technology) or Trianni mice (see, e.g., WO 2013/063391, WO 2017/035252 and WO 2017/136734).

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively.

The terms “antigen-binding domain” of an antibody (or simply “binding domain”) of an antibody or similar terms refer to one or more fragments of an antibody that retain the ability to specifically bind to an antigen complex. Examples of binding fragments encompassed within the term “antigen-binding portion” of an antibody include (i) Fab fragments, monovalent fragments consisting of the VL, VH, CL and CH domains; (ii) F(ab′)2 fragments, bivalent fragments comprising two Fab fragments linked by a disulfide bridge at the hinge region; (iii) Fd fragments consisting of the VH and CH domains; (iv) Fv fragments consisting of the VL and VH domains of a single arm of an antibody, (v) dAb fragments (Ward et al., (1989) Nature 341:544-546), which consist of a VH domain; (vi) isolated complementarity determining regions (CDR), and (vii) combinations of two or more isolated CDRs which may optionally be joined by a synthetic linker.

The “variable domain” (V domain) of an antibody mediates binding and confers antigen specificity of a particular antibody. However, the variability is not evenly distributed across the 110-amino acid span of the variable domains. Instead, the V regions consist of relatively invariant stretches called framework regions (FRs) of 15-30 amino acids separated by shorter regions of extreme variability referred to herein as “hypervariable regions” or CDRs that are each 9-12 amino acids long. As will be appreciated by those in the art, the exact numbering and placement of the CDRs can be different among different numbering systems. However, it should be understood that the disclosure of a variable heavy and/or variable light sequence includes the disclosure of the associated CDRs. Accordingly, the disclosure of each variable heavy region is a disclosure of the vhCDRs (e.g. vhCDR1, vhCDR2 and vhCDR3) and the disclosure of each variable light region is a disclosure of the vCDRs (e.g. vlCDR1, vlCDR2 and vlCDR3).

“Complementarity determining region” or “CDR” as the terms are used herein refer to short polypeptide sequences within the variable region of both heavy and light chain polypeptides that are primarily responsible for mediating specific antigen recognition. There are three CDRs (termed CDR1, CDR2, and CDR3) within each VL and each VH. Unless stated otherwise herein, CDR and framework regions are annotated according to the Kabat numbering scheme (Kabat E. A., et al., 1991, Sequences of proteins of Immunological interest, In: NIH Publication No. 91-3242, US Department of Health and Human Services, Bethesda, Md).

In other embodiments, the CDRs of an antibody can be determined according to MacCallum R M et al, (1996) J Mol Biol 262:732-745, herein incorporated by reference in its entirety or according to the IMGT numbering system as described in Lefranc M-P, (1999) The Immunologist 7:132-136 and Lefranc M-P et al, (1999) Nucleic Acids Res 27:209-212, each of which is herein incorporated by reference in its entirety. See also, e.g. Martin A. “Protein Sequence and Structure Analysis of Antibody Variable Domains,” in Antibody Engineering, Kontermann and Diibel, eds., Chapter 31, pp. 422-439, Springer-Verlag, Berlin (2001), herein incorporated by reference in its entirety. In other embodiments, the CDRs of an antibody can be determined according to the AbM numbering scheme, which refers to AbM hypervariable regions, which represent a compromise between the Kabat CDRs and Chothia structural loops, and are used by Oxford Molecular's AbM antibody modeling software (Oxford Molecular Group, Inc.), herein incorporated by reference in its entirety.

“Framework” or “framework region” or “FR” refers to variable domain residues other than hypervariable region (HVR) residues. The FR of a variable domain generally consists of four FR domains: FR1, FR2, FR3, and FR4.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda Md. (1991), Vols. 1-3. In one embodiment, for the VL, the subgroup is subgroup kappa I as in Kabat et al., supra. In one embodiment, for the VH, the subgroup is subgroup Ill as in Kabat et al., supra.

The “hinge region” is generally defined as stretching from 216-238 (EU numbering) or 226-251 (Kabat numbering) of human IgG1. The hinge can be further divided into three distinct regions, the upper, middle (e.g., core), and lower hinge.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md. (1991).

The term “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab)₂; diabodies; linear antibodies; single-chain antibody molecules (e.g., scFv). Papain digestion of antibodies produces two identical antigen-binding fragments, called “Fab” fragments, and a residual “Fc” fragment, a designation reflecting the ability to crystallize readily. The Fab fragment consists of an entire light (L) chain along with the variable region domain of the heavy (H) chain (VH), and the first constant domain of one heavy chain (CH1). Pepsin treatment of an antibody yields a single large F(ab)₂ fragment which roughly corresponds to two disulfide linked Fab fragments having divalent antigen-binding activity and is still capable of cross-linking antigen. Fab fragments differ from Fab′ fragments by having additional few residues at the carboxy terminus of the CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

“Fv” consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody.

“Single-chain Fv” also abbreviated as “sFv” or “scFv” are antibody fragments that comprise the VH and VL antibody domains connected into a single polypeptide chain. Preferably, the sFv polypeptide further comprises a polypeptide linker between the VH and VL domains which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

The terms “antigen-binding domain” of an antibody (or simply “binding domain”) of an antibody or similar terms refer to one or more fragments of an antibody that retain the ability to specifically bind to an antigen complex. Examples of binding fragments encompassed within the term “antigen-binding portion” of an antibody include (i) Fab fragments, monovalent fragments consisting of the VL, VH, CL and CH domains; (ii) F(ab′)2 fragments, bivalent fragments comprising two Fab fragments linked by a disulfide bridge at the hinge region; (iii) Fd fragments consisting of the VH and CH domains; (iv) Fv fragments consisting of the VL and VH domains of a single arm of an antibody, (v) dAb fragments (Ward et al., (1989) Nature 341:544-546), which consist of a VH domain; (vi) isolated complementarity determining regions (CDR), and (vii) combinations of two or more isolated CDRs which may optionally be joined by a synthetic linker.

The term “multispecific antibody” is used in the broadest sense and specifically covers an antibody comprising a heavy chain variable domain (VH) and a light chain variable domain (VL), where the VH-VL unit has polyepitopic specificity (e.g., is capable of binding to two different epitopes on one biological molecule or each epitope on a different biological molecule). Such multispecific antibodies include, but are not limited to, full-length antibodies, antibodies having two or more VL and VH domains, bispecific diabodies and triabodies. “Polyepitopic specificity” refers to the ability to specifically bind to two or more different epitopes on the same or different target(s).

“Dual specificity” or “bispecificity” refers to the ability to specifically bind to two different epitopes on the same or different target(s). However, in contrast to bispecific antibodies, dual-specific antibodies have two antigen-binding arms that are identical in amino acid sequence and each Fab arm is capable of recognizing two antigens. Dual-specificity allows the antibodies to interact with high affinity with two different antigens as a single Fab or IgG molecule. According to one embodiment, the multispecific antibody in an IgG1 form binds to each epitope with an affinity of 5 μM to 0.001 pM, 3 μM to 0.001 pM, 1 μM to 0.001 pM, 0.5 μM to 0.001 pM or 0.1 μM to 0.001 pM. “Monospecific” refers to the ability to bind only one epitope. Multi-specific antibodies can have structures similar to full immunoglobulin molecules and include Fc regions, for example IgG Fc regions. Such structures can include, but are not limited to, IgG-Fv, IgG-(scFv)₂, DVD-Ig, (scFv)₂-(scFv)₂-Fc and (scFv)₂-Fc-(scFv)₂. In case of IgG-(scFv) 2, the scFv can be attached to either the N-terminal or the C-terminal end of either the heavy chain or the light chain.

As used herein, the term “bispecific antibodies” refers to monoclonal, often human or humanized, antibodies that have binding specificities for at least two different antigens. In the disclosure, one of the binding specificities can be directed towards CLDN6, the other can be for any other antigen, e.g., for a cell-surface protein, receptor, receptor subunit, tissue-specific antigen, virally derived protein, virally encoded envelope protein, bacterially derived protein, or bacterial surface protein, etc.

As used herein, the term “diabodies” refers to bivalent antibodies comprising two polypeptide chains, in which each polypeptide chain includes VH and VL domains joined by a linker that is too short (e.g., a linker composed of five amino acids) to allow for intramolecular association of VH and VL domains on the same peptide chain. This configuration forces each domain to pair with a complementary domain on another polypeptide chain so as to form a homodimeric structure. Accordingly, the term “triabodies” refers to trivalent antibodies comprising three peptide chains, each of which contains one VH domain and one VL domain joined by a linker that is exceedingly short (e.g., a linker composed of 1-2 amino acids) to permit intramolecular association of VH and VL domains within the same peptide chain.

The term an “isolated antibody” when used to describe the various antibodies disclosed herein, means an antibody that has been identified and separated and/or recovered from a cell or cell culture from which it was expressed. An isolated antibody or antibody fragment may include variants of the antibody or antibody fragment having one or more post-translational modifications (e.g., C-terminal lysine clipping) that arise during production, purification, and/or storage of the antibody or antibody fragment. Contaminant components of its natural environment are materials that would typically interfere with diagnostic or therapeutic uses for the polypeptide, and can include enzymes, hormones, and other proteinaceous or non-proteinaceous solutes. In some embodiments, an isolated antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC) approaches. For a review of methods for assessment of antibody purity, see, for example, Flatman et al., J. Chromatogr. B 848:79-87 (2007). In a preferred embodiment, the antibody will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain.

With regard to the binding of an antibody to a target molecule, the term “specific binding” or “specifically binds to” or is “specific for” a particular polypeptide or an epitope on a particular polypeptide target means binding that is measurably different from a non-specific interaction. Specific binding can be measured, for example, by determining binding of a molecule compared to binding of a control molecule. For example, specific binding can be determined by competition with a control molecule that is similar to the target, for example, an excess of non-labeled target. In this case, specific binding is indicated if the binding of the labeled target to a probe is competitively inhibited by excess unlabeled target. The term “specific binding” or “specifically binds to” or is “specific for” a particular polypeptide or an epitope on a particular polypeptide target as used herein can be exhibited, for example, by a molecule having a Kd for the target of 10-4 M or lower, alternatively 10-5 M or lower, alternatively 10-6 M or lower, alternatively 10-7 M or lower, alternatively 10-8 M or lower, alternatively 10-9 M or lower, alternatively 10-10 M or lower, alternatively 10-11 M or lower, alternatively 10-12 M or lower or a Kd in the range of 10-4 M to 10-6 M or 10-6 M to 10-10 M or 10-7 M to 10-9 M. As will be appreciated by the skilled artisan, affinity and KD values are inversely related. A high affinity for an antigen is measured by a low KD value. In one embodiment, the term “specific binding” refers to binding where a molecule binds to CLDN6 or to a CLDN6 epitope without substantially binding to any other polypeptide or polypeptide epitope.

As used herein the term “binds CLDN6” refers to the ability of an antibody, or antigen-binding fragment to recognize and bind endogenous human CLDN6 as it occurs on the surface of normal or malignant cells or on the surface of recombinant host cells engineered to overexpress CLDN6.

The term “affinity,” as used herein, means the strength of the binding of an antibody to an epitope. The affinity of an antibody is given by the dissociation constant Kd, defined as [Ab]×[Ag]/[Ab-Ag], where [Ab-Ag] is the molar concentration of the antibody-antigen complex, [Ab] is the molar concentration of the unbound antibody and [Ag] is the molar concentration of the unbound antigen. The affinity constant Ka is defined by 1/Kd. Methods for determining the affinity of mAbs can be found in Harlow, et al., Antibodies: A Laboratory Manual, Cold Spring Harbor Laboratory Press, Cold Spring Harbor, N. Y., 1988), Coligan et al., eds., Current Protocols in Immunology, Greene Publishing Assoc. and Wiley Interscience, N.Y., (1992, 1993), and Muller, Meth. Enzymol. 92:589-601 (1983), which references are entirely incorporated herein by reference. One standard method well known in the art for determining the affinity of mAbs is the use of surface plasmon resonance (SPR) screening (such as by analysis with a BIAcore™ SPR analytical device).

An “epitope” is a term of art that indicates the site or sites of interaction between an antibody and its antigen(s). As described by (Janeway, C, Jr., P. Travers, et al. (2001). Immunobiology: the immune system in health and disease. Part II, Section 3-8. New York, Garland Publishing, Inc.): “An antibody generally recognizes only a small region on the surface of a large molecule such as a protein . . . [Certain epitopes] are likely to be composed of amino acids from different parts of the [antigen] polypeptide chain that have been brought together by protein folding. Antigenic determinants of this kind are known as conformational or discontinuous epitopes because the structure recognized is composed of segments of the protein that are discontinuous in the amino acid sequence of the antigen but are brought together in the three-dimensional structure. In contrast, an epitope composed of a single segment of polypeptide chain is termed a continuous or linear epitope” (Janeway, C. Jr., P. Travers, et al. (2001). Immunobiology: the immune system in health and disease. Part II, Section 3-8. New York, Garland Publishing, Inc.).

The term “KD”, as used herein, refers to the equilibrium dissociation constant, which is obtained from the ratio of kd to ka (e.g., kd/ka) and is expressed as a molar concentration (M). KD values for antibodies can be determined using methods well established in the art. Preferred methods for determining the KD of an antibody include biolayer interferometry (BLI) analysis, preferably using a Fortebio Octet RED device, surface plasmon resonance, preferably using a biosensor system such as a BIACORE® surface plasmon resonance system, or flow cytometry and Scatchard analysis.

“EC₅₀” with respect to an agent and a particular activity (e.g. binding to a cell, inhibition of enzymatic activity, activation or inhibition of an immune cell), refers to the efficient concentration of the agent which produces 50% of its maximum response or effect with respect to such activity. “EC₁₀₀” with respect to an agent and a particular activity refers to the efficient concentration of the agent which produces its substantially maximum response with respect to such activity.

As used herein the term “antibody-drug conjugate” (ADC) refers to immunoconjugates consisting of recombinant monoclonal antibodies covalently linked to cytotoxic agents (known as payloads) via synthetic linkers. Immunoconjugates (Antibody-drug conjugates, ADCs) are a class of highly potent antibody-based cancer therapeutics. ADCs consist of recombinant monoclonal antibodies covalently linked to cytotoxic agents (known as payloads) via synthetic linkers. ADCs combine the specificity of monoclonal antibodies and the potency of small-molecule chemotherapy drugs, and facilitate the targeted delivery of highly cytotoxic small molecule drug moieties directly to tumor cells.

As used herein the term “endocytosis” refers to the process where eukaryotic cells internalize segments of the plasma membrane, cell-surface receptors, and components from the extracellular fluid. Endocytosis mechanisms include receptor-mediated endocytosis. The term “receptor-mediated endocytosis” refers to a biological mechanism by which a ligand, upon binding to its target, triggers membrane invagination and pinching, gets internalized and delivered into the cytosol or transferred to appropriate intracellular compartments.

The term “bystander effect” refers to target-cell mediated killing of healthy cells adjacent to tumor cells targeted for by an antibody drug conjugate. The bystander effect is generally caused by cellular efflux of hydrophobic cytotoxic drugs, capable of diffusing out of an antigen-positive target cell and into adjacent antigen-negative healthy cells. The presence or absence of the bystander effect can be attributed to aspects of the linker and conjugation chemistries used to produce an immunoconjugate.

The term “effector functions,” deriving from the interaction of an antibody Fc region with certain Fc receptors, include but are not necessarily limited to Clq binding, complement dependent cytotoxicity (CDC), Fc receptor binding, FcyR-mediated effector functions such as ADCC, antibody dependent cell-mediated phagocytosis (ADCP), T cell dependent cellular cytotoxicity (TCDD) and down regulation of a cell surface receptor. Such effector functions generally require the Fc region to be combined with an antigen binding domain (e.g., an antibody variable domain).

As used herein the terms “antibody-based immunotherapy” and “immunotherapies” are used to broadly refer to any form of therapy that relies on the targeting specificity of an anti-CLDN6 antibody, bispecific molecule, antigen-binding domain, or fusion protein comprising an anti-CLDN6 antibody or antibody fragments or CDRs thereof, to mediate a direct or indirect effect on a CLDN6 expressing cell. The terms are meant to encompass methods of treatment using naked antibodies, bispecific antibodies (including T-cell engaging, NK cell engaging and other immune cell/effector cell engaging formats) antibody drug conjugates, cellular therapies using T cells (CAR-T) or NK cells (CAR-NK) engineered to comprise an anti-CLDN6 chimeric antigen receptor and oncolytic viruses comprising a CLDN6 specific binding agent, and gene therapies by delivering the antigen binding sequences of the anti-CLDN6 antibodies and express the corresponding antibody fragments in vivo.

### Claudin Protein Family

Claudins are integral membrane proteins comprising a major structural protein of tight junctions, the most apical cell-cell adhesion junction in polarized cell types such as those found in epithelial or endothelial cell sheets.

The claudin family of proteins in humans is comprised of at least 24 members, ranging in size from 22-34 kDa. All claudins possess a tetraspanin topology in which both protein termini are located on the intracellular face of the membrane, resulting in the formation of two extracellular (EC) loops, EC1 and EC2. Typically, EC1 is about 50-60 amino acids in size and EC2 is smaller than EC1 and usually comprises approximately 25 amino acids. The EC loops mediate head-to-head homophilic, and for certain combinations of claudins, heterophilic interactions that lead to formation of tight junctions.

### Claudin-6

Claudin-6 (CLDN6) is generally expressed in humans as a 220-amino acid precursor protein; the first 21 amino acids of which constitute the signal peptide. The amino acid sequence of the CLDN6 precursor protein is publicly available at the National Center for Biotechnology Information (NCBI) website as NCBI Reference Sequence NP 067018.2 and is provided herein as SEQ ID NO: 17.

Expression CLDN6 is highly expressed in germ cell tumors, including seminomas, embryonal carcinomas and yolk sac tumors, as well as in some cases of gastric adenocarcinomas, lung adenocarcinomas, ovarian adenocarcinomas, and endometrial carcinomas (Ushiku T, et al., Histopathology 61 (6): 1043-1056, 2012, Hewitt K J, Agarwal R, Morin P J. The claudin gene family: expression in normal and neoplastic tissues. BMC Cancer 2006; 6; 186; Micke, P. et al. (2014) Aberrantly activated Claudin-6 and 18.2 as potential therapy targets in non-small-cell lung cancer. Int. J. Cancer 135, 2206-2214; Lal-Nag, M. et al. (2012) Claudin-6: a novel receptor for CPE-mediated cytotoxicity in ovarian cancer. Oncogenesis 1, e33; Ben-David, U. et al. (2013) Immunologic and chemical targeting of the tight junction protein Claudin-6 eliminates tumorigenic human pluripotent stem cells. Nat. Commun. 4, 1992).

Human CLDN6 protein is very closely related to the human CLDN9 protein sequence in the extracellular domains (ECD), with >98% identity in ECD1 and >91% identity in ECD2. Human CLDN4 is also closely related to human CLDN6 in the ECD sequences, with >84% identity in ECD1 and >78% identity in ECD2. Monoclonal antibody (MAb) discovery against CLDN6 has been encumbered by the high homology of endogenously expressed Claudin-9 (CLDN9), which varies from CLDN6 by only 3 amino acids (2 in ECD1 and 1 in ECD2) in their extracellular domains. Deduced cynomolgus monkey protein ECD sequences for CLDN4, CLDN6, and CLDN9 proteins are 100% identical to the respective human ECD sequences. Accordingly, it is expected that the disclosed anti-human CLDN6 antibodies and fragments are cross-reactive with cynomolgus monkey CLDN6 (data not shown). In addition, the Claudin-6 gene is highly conserved among different species, for example, human and murine genes exhibit 88% homology at DNA and protein level.

### Targeting CLDN6 for Cancer Treatment

In the last few years, it became more and more convincing that tight junctions play a role in proliferation, transformation and metastasis of cancer cells. Dysregulation of claudins leads to disruption of tight junctions in epithelial cells which in turn results in loss of cell polarity and impairment of the epithelial integrity. The overexpression of CLDN6 by tumor cells may be linked to disregulate localization of claudins as a consequence of the dedifferentiation of tumor cells, or the requirement of rapidly growing cancerous tissues to efficiently absorb nutrients within a tumor mass with abnormal vascularization (Morin P J., Cancer Res. 1; 65 (21): 9603-6, 2005). Decreased cell-cell adhesion and increased mobility of cancer cells are suggested to be main events of epithelial to mesenchymal transition (EMT), an important step in cancer progression and metastasis.

### Anti-CLDN6 Antibodies

The disclosed anti-CLDN6 antibodies (NR.N6.Ab1, NR.N6.Ab2, NR.N6.Ab3, NR.N6.Ab4, NR.N6.Ab5 and NR.N6.Ab6) selectively bind to human CLDN6 or to human CLDN6/9. These antibodies and fragments thereof are characterized by unique sets of CDR sequences for CLDN6 and are useful in cancer immunotherapy as monotherapy or in combination with other anti-cancer agents.

In some embodiments, the anti-CLDN6 antibodies or antibody fragments thereof exhibit one or more of the following structural and functional characteristics, alone or in combination: (a) bind to cells expressing human CLDN6 on their cell surface; (b) selectively bind to Claudin-6 CHO-K1 cells with a signal (e.g., MFI) that is approximately 20 to 25-fold greater than the binding activity of an isotype control, or 20 to 30-fold greater than the binding activity of an isotype control antibody for CLDN6 expressed on Claudin-6-HEK293 cell; (c) bind to Claudin-6 (Claudin-6 CHO-K1 cells) and Claudin-9 (HEK293 cells) expressing cells equally with a signal that it at approximately 25 to 60 fold greater than the binding activity of an isotype control antibody; (d) binds weakly or not at all to cells expressing CLDN3, CLDN4; (e) bind to NEC8 cells endogenously expressing Claudin-6, but do not bind to NEC8 cells with a knockout of the Claudin 6 gene NEC8; (f) optionally cross-react with murine CLDN6; (g) are efficiently internalized from the surface of Claudin-6 positive cells after binding and inducing endocytosis-mediated cell cytotoxicity in NEC8 cells endogenously expressing Claudin-6; and (h) exhibit one or more immune effector functions against a cell carrying CLDN6 in its native conformation, wherein the one or more immune effector functions is selected antibody-dependent cell-mediated cytotoxicity (ADCC), T-cell dependent cellular cytotoxicity (TDCC), complement dependent cytotoxicity (CDC), or antibody-dependent cellular phagocytosis (ADCP).

In an embodiment, the anti-CLDN6 antibodies or antibody fragments thereof comprise a VH having a set of CDRs (HCDR1, HCDR2, and HCDR3) disclosed in Table 1. For example, the anti-CLDN6 antibodies or antibody fragments thereof may comprise a set of CDRs corresponding to those CDRs in one of the anti-CLDN6 antibodies disclosed in Table 1 (e.g., the CDRs of the NR.N6.Ab1).

In another embodiment, the anti-CLDN6 antibodies comprise a VL having a set of CDRs (LCDR1, LCDR2, and LCDR3) as disclosed in Table 2. For example, the anti-CLDN6 antibodies or antibody fragments thereof may comprise a set of CDRs corresponding to those CDRs in one of the anti-CLDN6 antibodies disclosed in Table 2 (e.g., the CDRs of the NR.N6.Ab2).

In an alternative embodiment, the anti-CLDN6 antibodies or antibody fragments thereof comprise a VH having a set of CDRs (HCDR1, HCDR2, and HCDR3) as disclosed in Table 1, and a VL having a set of CDRs (LCDR1, LCDR2, and LCDR3) as disclosed in Table 2.

In an embodiment, the antibody may be a monoclonal, chimeric, humanized or human antibody, or antigen-binding portions thereof that specifically binds to human CLDN6. In one embodiment, the anti-CLDN6 antibody or antibody fragment thereof comprises all six of the CDR regions of the NR.N6.Ab1 or NR.N6.Ab2 antibody formatted as a chimeric or a humanized antibody.

TABLE 1
CDR Sequences of Anti-CLDN6 Antibody Variable Heavy Chains

| Anti-CLDN6 | | | |
|---|---|---|---|
| Ab | HCDR1 | HCDR2 | HCDR3 |
| NR.N6.Ab1 | SEQ ID NO: 5 | SEQ ID NO: 6 | SEQ ID NO: 7 |
| NR.N6.Ab2 | SEQ ID NO: 11 | SEQ ID NO: 12 | SEQ ID NO: 13 |
| NR.N6.Ab3 | SEQ ID NO: 32 | SEQ ID NO: 33 | SEQ ID NO: 34 |
| NR.N6.Ab4 | SEQ ID NO: 38 | SEQ ID NO: 39 | SEQ ID NO: 40 |
| NR.N6.Ab5 | SEQ ID NO: 38 | SEQ ID NO: 44 | SEQ ID NO: 40 |
| NR.N6.Ab6 | SEQ ID NO: 46 | SEQ ID NO: 47 | SEQ ID NO: 48 |

TABLE 2
CDR Sequences of Anti-CLDN6 Variable Light Chains

| Anti-CLDN6 | | | |
|---|---|---|---|
| Ab | LCDR1 | LCDR2 | LCDR3 |
| NR.N6.Ab1 | SEQ ID NO: 8 | SEQ ID NO: 9 | SEQ ID NO: 10 |
| NR.N6.Ab2 | SEQ ID NO: 14 | SEQ ID NO: 15 | SEQ ID NO: 16 |
| NR.N6.Ab3 | SEQ ID NO: 35 | SEQ ID NO: 36 | SEQ ID NO: 37 |
| NR.N6.Ab4 | SEQ ID NO: 41 | SEQ ID NO: 42 | SEQ ID NO: 43 |
| NR.N6.Ab5 | SEQ ID NO: 41 | SEQ ID NO: 45 | SEQ ID NO: 43 |
| NR.N6.Ab6 | SEQ ID NO: 49 | SEQ ID NO: 50 | SEQ ID NO: 51 |

In an embodiment, the anti-CLDN6 antibodies or antibody fragments thereof comprise a VH having a set of complementarity-determining regions (CDR1, CDR2, and CDR3) selected from the group consisting of:
(i) CDR1: SEQ ID NO: 5, CDR2: SEQ ID NO: 6, CDR3: SEQ ID NO: 7;
(ii) CDR1: SEQ ID NO: 11, CDR2: SEQ ID NO: 12, CDR3: SEQ ID NO: 13;
(iii) CDR1: SEQ ID NO: 32, CDR2: SEQ ID NO: 33, CDR3: SEQ ID NO: 34;
(iv) CDR1: SEQ ID NO: 38, CDR2: SEQ ID NO: 39, CDR3: SEQ ID NO: 40;
(v) CDR1: SEQ ID NO: 38, CDR2: SEQ ID NO: 44, CDR3: SEQ ID NO: 40; and
(vi) CDR1: SEQ ID NO: 46, CDR2: SEQ ID NO: 47, CDR3: SEQ ID NO: 48.

In an embodiment, the anti-CLDN6 antibodies or antibody fragments thereof comprise a VL having a set of complementarity-determining regions (CDR1, CDR2, and CDR3) selected from the group consisting of:
(i) CDR1: SEQ ID NO: 8, CDR2: SEQ ID NO: 9, CDR3: SEQ ID NO: 10;
(ii) CDR1: SEQ ID NO: 14, CDR2: SEQ ID NO: 15, CDR3: SEQ ID NO: 16
(iii) CDR1: SEQ ID NO: 35, CDR2: SEQ ID NO: 36, CDR3: SEQ ID NO: 37;
(iv) CDR1: SEQ ID NO: 41, CDR2: SEQ ID NO: 42, CDR3: SEQ ID NO: 43;
(v) CDR1: SEQ ID NO: 41, CDR2: SEQ ID NO: 45, CDR3: SEQ ID NO: 43; and
(vi) CDR1: SEQ ID NO: 49, CDR2: SEQ ID NO: 50, CDR3: SEQ ID NO: 51.

In another embodiment, the anti-CLDN6 antibodies or antibody fragments thereof comprise:
(a) a VH having a set of complementarity-determining regions (CDR1, CDR2, and CDR3) selected from the group consisting of:
   (i) CDR1: SEQ ID NO: 5, CDR2: SEQ ID NO: 6, CDR3: SEQ ID NO: 7; and
   (ii) CDR1: SEQ ID NO: 11, CDR2: SEQ ID NO: 12, CDR3: SEQ ID NO: 13;
   (iii) CDR1: SEQ ID NO: 32, CDR2: SEQ ID NO: 33, CDR3: SEQ ID NO: 34;
   (iv) CDR1: SEQ ID NO: 38, CDR2: SEQ ID NO: 39, CDR3: SEQ ID NO: 40;
   (v) CDR1: SEQ ID NO: 38, CDR2: SEQ ID NO: 44, CDR3: SEQ ID NO: 40; and
   (vi) CDR1: SEQ ID NO: 46, CDR2: SEQ ID NO: 47, CDR3: SEQ ID NO: 48;
   and
(b) a VL having a set of complementarity-determining regions (CDR1, CDR2, and CDR3) selected from the group consisting of:
   (i) CDR1: SEQ ID NO: 8, CDR2: SEQ ID NO: 9, CDR3: SEQ ID NO: 10;
   (ii) CDR1: SEQ ID NO: 14, CDR2: SEQ ID NO: 15, CDR3: SEQ ID NO: 16;
   (iii) CDR1: SEQ ID NO: 35, CDR2: SEQ ID NO: 36, CDR3: SEQ ID NO: 37;
   (iv) CDR1: SEQ ID NO: 41, CDR2: SEQ ID NO: 42, CDR3: SEQ ID NO: 43;
   (v) CDR1: SEQ ID NO: 41, CDR2: SEQ ID NO: 45, CDR3: SEQ ID NO: 43; and
   (vi) CDR1: SEQ ID NO: 49, CDR2: SEQ ID NO: 50, CDR3: SEQ ID NO: 51.

In an embodiment, the antibodies comprise a combination of a VH and a VL having a set of complementarity-determining regions (CDR1, CDR2 and CDR3) selected from the group consisting of:
(i) VH: CDR1: SEQ ID NO: 5, CDR2: SEQ ID NO: 6, CDR3: SEQ ID NO: 7, VL: CDR1: SEQ ID NO: 8, CDR2: SEQ ID NO: 9, CDR3: SEQ ID NO: 10,
ii) VH: CDR1: SEQ ID NO: 11, CDR2: SEQ ID NO: 12, CDR3: SEQ ID NO: 13, VL: CDR1: SEQ ID NO: 14, CDR2: SEQ ID NO: 15, CDR3: SEQ ID NO: 16,
iii) VH: CDR1: SEQ ID NO: 32, CDR2: SEQ ID NO: 33, CDR3: SEQ ID NO: 34, VL: CDR1: SEQ ID NO: 35, CDR2: SEQ ID NO: 36, CDR3: SEQ ID NO:37,
iv) VH: CDR1: SEQ ID NO: 38, CDR2: SEQ ID NO: 39, CDR3: SEQ ID NO: 40, VL: CDR1: SEQ ID NO: 41, CDR2: SEQ ID NO: 42, CDR3: SEQ ID NO: 43,
v) VH: CDR1: SEQ ID NO: 38, CDR2: SEQ ID NO: 44, CDR3: SEQ ID NO: 40, VL: CDR1: SEQ ID NO: 41, CDR2: SEQ ID NO: 45, CDR3: SEQ ID NO: 43, and
vi) VH: CDR1: SEQ ID NO: 46, CDR2: SEQ ID NO: 47, CDR3: SEQ ID NO: 48, VL: CDR1: SEQ ID NO: 49, CDR2: SEQ ID NO: 50, CDR3: SEQ ID NO: 51.

In an embodiment, the anti-CLDN6 antibodies or antibody fragments thereof comprise a variable heavy chain sequence selected from: SEQ ID NOs: 1, 3, 23, 24, 26, 28 and 30, and/or a variable light chain sequence selected from SEQ ID NOs: 2, 4, 25, 27, 29 and 31.

In an embodiment, the anti-CLDN6 antibodies or antibody fragments thereof comprise a pair of variable heavy chain and variable light chain sequences, selected from the following combinations: a variable heavy chain sequence comprising SEQ ID NO: 1 and a variable light chain sequence comprising SEQ ID NO: 2; a variable heavy chain sequence comprising SEQ ID NO: 3 and a variable light chain sequence comprising SEQ ID NO: 4; a variable heavy chain sequence comprising SEQ ID NO: 23 and a variable light chain sequence comprising SEQ ID NO: 2; a variable heavy chain sequence comprising SEQ ID NO: 24 and a variable light chain sequence comprising SEQ ID NO: 25; a variable heavy chain sequence comprising SEQ ID NO: 26 and a variable light chain sequence comprising SEQ ID NO: 27; a variable heavy chain sequence comprising SEQ ID NO: 28 and a variable light chain sequence comprising SEQ ID NO: 29; and a variable heavy chain sequence comprising SEQ ID NO: 30 and a variable light chain sequence comprising SEQ ID NO: 31. The skilled person will further understand that the variable light and variable heavy chains may be independently selected, or mixed and matched, to prepare an anti-CLDN6 antibody comprising a combination of variable heavy and variable light chain that is distinct from the pairings identified above.

In an alternative embodiment, the anti-CLDN6 antibodies or antibody fragments thereof comprise a pair of variable heavy chain and variable light chain sequences, selected from the following combinations: a variable heavy chain sequence that is 90%, 95%, or 99% identical to SEQ ID NO: 1 and a variable light chain sequence that is 90%, 95%, or 99% identical to SEQ ID NO: 2; a variable heavy chain sequence that is 90%, 95%, or 99% identical to SEQ ID NO: 3 and a variable light chain sequence that is 90%, 95%, or 99% identical to SEQ ID NO: 4; a variable heavy chain sequence that is 90%, 95%, or 99% identical to SEQ ID NO: 23 and a variable light chain sequence that is 90%, 95%, or 99% identical to SEQ ID NO: 2; a variable heavy chain sequence that is 90%, 95%, or 99% identical to SEQ ID NO: 24 and a variable light chain sequence that is 90%, 95%, or 99% identical to SEQ ID NO: 25; a variable heavy chain sequence that is 90%, 95%, or 99% identical to SEQ ID NO: 26 and a variable light chain sequence that is 90%, 95%, or 99% identical to SEQ ID NO: 27; a variable heavy chain sequence that is 90%, 95%, or 99% identical to SEQ ID NO: 28 and a variable light chain sequence that is 90%, 95%, or 99% identical to SEQ ID NO: 29; and a variable heavy chain sequence that is 90%, 95%, or 99% identical to SEQ ID NO: 30 and a variable light chain sequence that is 90%, 95%, or 99% identical to SEQ ID NO: 31. The skilled person will further understand that the variable light and variable heavy chains may be independently selected, or mixed and matched, to prepare an anti-CLDN6 antibody comprising a combination of variable heavy and variable light chain that is distinct from the pairings identified above.

In some embodiments, the antibody is a full-length antibody. In other embodiments, the antibody is an antibody fragment including, for example, an antibody fragment selected from the group consisting of: Fab, Fab′, F(ab)₂, Fv, domain antibodies (dAbs), and complementarity determining region (CDR) fragments, single-chain antibodies (scFv), chimeric antibodies, diabodies, triabodies, tetrabodies, miniantibodies, and polypeptides that contain at least a portion of an immunoglobulin that is sufficient to confer CLDN6 selective binding to the polypeptide.

In some embodiments, a variable region domain of an anti-CLDN6 antibody disclosed herein may be covalently attached at a C-terminal amino acid to at least one other antibody domain or a fragment thereof. Thus, for example, a VH domain that is present in the variable region domain may be linked to an immunoglobulin CHI domain, or a fragment thereof. Similarly, a VL domain may be linked to a CK domain or a fragment thereof. In this way, for example, the antibody may be a Fab fragment wherein the antigen binding domain contains associated VH and VL domains covalently linked at their C-termini to a CHI and CK domain, respectively. The CHI domain may be extended with further amino acids, for example to provide a hinge region or a portion of a hinge region domain as found in a Fab fragment, or to provide further domains, such as antibody CH2 and CH3 domains.

Thus, in one embodiment, the antibody fragment comprises at least one CDR as described herein. The antibody fragment may comprise at least two, three, four, five, or six CDRs as described herein. The antibody fragment further may comprise at least one variable region domain of an antibody described herein. The variable region domain may be of any size or amino acid composition and will generally comprise at least one CDR sequence responsible for binding to human CLDN6, for example, CDR-H1, CDR-H2, CDR-H3, CDR-L1, CDR-L2, and/or CDR-L3 as described herein, and which is adjacent to or in frame with one or more framework sequences.

In some embodiments, the anti-CLDN6 antibody is a monoclonal antibody. In some embodiments, the anti-CLDN6 antibody is a human antibody. In alternative embodiments, the anti-CLDN6 antibody is a murine antibody. In some embodiments, the anti-CLDN6 antibody is a chimeric antibody, a bispecific antibody, or a humanized antibody.

In some embodiments, the anti-CLDN6 antibodies or antibody fragments thereof comprise one or more conservative amino acid substitutions. A person of skill in the art will recognize that a conservative amino acid substitution is a substitution of one amino acid with another amino acid that has similar structural or chemical properties, such as, for example, a similar side chain. Exemplary conservative substitutions are described in the art, for example, in Watson et al., Molecular Biology of the Gene, The Benjamin/Cummings Publication Company, 4th Ed. (1987).

“Conservative modifications” refer to amino acid modifications that do not significantly affect or alter the binding characteristics of the antibody containing the amino acid sequences. Conservative modifications include amino acid substitutions, additions and deletions. Conservative substitutions are those in which the amino acid is replaced with an amino acid residue having a similar side chain. The families of amino acid residues having similar side chains are well defined and include amino acids with acidic side chains (e.g., aspartic acid, glutamic acid), basic side chains (e.g., lysine, arginine, histidine), nonpolar side chains (e.g., alanine, valine, leucine, isoleucine, proline, phenylalanine, methionine), uncharged polar side chains (e.g., glycine, asparagine, glutamine, cysteine, serine, threonine, tyrosine, tryptophan), aromatic side chains (e.g., phenylalanine, tryptophan, histidine, tyrosine), aliphatic side chains (e.g., glycine, alanine, valine, leucine, isoleucine, serine, threonine), amide (e.g., asparagine, glutamine), beta-branched side chains (e.g., threonine, valine, isoleucine) and sulfur-containing side chains (cysteine, methionine). Furthermore, any native residue in the polypeptide may also be substituted with alanine, as has been previously described for alanine scanning mutagenesis (MacLennan et al. (1998) Acta Physiol Scan Suppl 643:55-67; Sasaki et al. (1998) Adv Biophys 35:1-24). Amino acid substitutions to the antibodies of the disclosure may be made by known methods for example by PCR mutagenesis (U.S. Pat. No. 4,683,195).

In some embodiments, the anti-CLDN6 antibodies or antibody fragments thereof comprise a variable heavy chain sequence that comprises an amino acid sequence with at least about 95%, about 96%, about 97%, about 98%, or about 99%, sequence identity to the amino acid sequence set forth in SEQ ID NOs: 1, 3, 23, 24, 26, 28 or 30. In other embodiments, the anti-CLDN6 antibodies or antibody fragments thereof retains the binding and/or functional activity of an anti-CLDN6 antibody or antibody fragment thereof that comprises the variable heavy chain sequence of SEQ ID Nos: 1, 3, 23, 24, 26, 28 or 30. In still further embodiments, the anti-CLDN6 antibodies or antibody fragments thereof comprise the variable heavy chain sequence of SEQ ID Nos: 1, 3, 23, 24, 26, 28 or 30 and have one or more conservative amino acid substitutions, e.g., 1, 2, 3, 4, 5, 1-2, 1-3, 1-4 or 1-5 conservative amino acid substitutions in the heavy chain variable sequence. In yet further embodiments, the one or more conservative amino acid substitutions fall within one or more framework regions in SEQ ID NOs: 1, 3, 23, 24, 26, 28 or 30 (based on the numbering system of Kabat).

In particular embodiments, the anti-CLDN6 antibody or antibody fragment thereof comprises a variable heavy chain sequence with at least about 95%, about 96%, about 97%, about 98%, or about 99% sequence identity to the anti-CLDN6 heavy chain variable region sequence set forth in SEQ ID NOs: 1, 3, 23, 24, 26, 28 or 30, comprises one or more conservative amino acid substitutions in a framework region (based on the numbering system of Kabat), and retains the binding and/or functional activity of an anti-CLDN6 antibody or antibody fragment thereof that comprises a variable heavy chain sequence as set forth in SEQ ID NOs: 1, 3, 23, 24, 26, 28 or 30 and a variable light chain sequence as set forth in SEQ ID NOs: 2, 4, 25, 27, 29 or 31.

In some embodiments, the anti-CLDN6 antibodies or antibody fragments thereof comprise a variable light chain sequence that comprises an amino acid sequence with at least about 95%, about 96%, about 97%, about 98%, or about 99%, sequence identity to the amino acid sequence set forth in SEQ ID NOs: 2, 4, 25, 27, 29 or 31. In other embodiments, the anti-CLDN6 antibodies or antibody fragments thereof retains the binding and/or functional activity of an anti-CLDN6 antibody or antibody fragment thereof that comprises the variable light chain sequence of SEQ ID Nos: 2, 4, 25, 27, 29 or 31. In still further embodiments, the anti-CLDN6 antibodies or antibody fragments thereof comprise the variable light chain sequence of SEQ ID Nos: 2, 4, 25, 27, 29 or 31 and have one or more conservative amino acid substitutions, e.g., 1, 2, 3, 4, 5, 1-2, 1-3, 1-4 or 1-5 conservative amino acid substitutions in the light chain variable sequence. In yet further embodiments, the one or more conservative amino acid substitutions fall within one or more framework regions in SEQ ID NOs: 2, 4, 25, 27, 29 or 31 (based on the numbering system of Kabat).

In particular embodiments, the anti-CLDN6 antibody or antibody fragment thereof comprises a variable light chain sequence with at least about 95%, about 96%, about 97%, about 98%, or about 99% sequence identity to the anti-CLDN6 light chain variable region sequence set forth in SEQ ID NOs: 2, 4, 25, 27, 29 or 31, comprises one or more conservative amino acid substitutions in a framework region (based on the numbering system of Kabat), and retains the binding and/or functional activity of an anti-CLDN6 antibody or antibody fragment thereof that comprises a variable heavy chain sequence as set forth in SEQ ID NOs: 1, 3, 23, 24, 26, 28 or 30 and a variable light chain sequence as set forth in SEQ ID NOs: 2, 4, 25, 27, 29 or 31.

The therapeutic value of the antibodies of the disclosure can be enhanced by conjugation to a cytotoxic drug or agent that improves its effectiveness and potency. In some embodiments the antibody is an antibody drug conjugate (ADC) comprising a CLDN6-specific antibody coupled to a cytotoxic effector agent such as a radioisotope, a drug, or a cytotoxin.

The anti-CLDN6 antibodies of the disclosure can also be used for developing antibody-based immunotherapeutics that rely on CLDN6 or CLDN6/9 selective binding to direct patient effector cells (e.g., T-cells or NK cells) to tumors including bispecific T cell engaging antibodies, or bispecific molecules that redirect NK cells, or cell therapies, such as CAR-T therapy.

In exemplary aspects, the disclosed anti-CLN6 antibodies or fragments thereof may be incorporated into an antigen-binding protein in the form of a bispecific antibody that is capable of binding two different and distinct antigens. Over fifty formats of bispecific antigen-binding proteins are known in the art, some of which are described in Kontermann and Brinkmann, Drug Discovery Today 20 (7): 838-847 (2015); Zhang et al., Exp Hematol Oncol 6:12 (2017); Spiess et al., Mol Immunol.; 67 (2 Pt A): 95-106 (2015). In one exemplary aspect the anti-CDLN6 antigen binding protein component of the bispecific antibody is a full-length antibody. In an alternative embodiment, the bispecific antigen-binding protein comprises an anti-CLDN6 scFv comprising the LC and HC variable regions of any of the presently disclosed antibodies.

In various aspects, the antigen binding fragment is based on the heavy chain variable region and in other aspects, the antigen binding fragment is based on the light chain variable region. In exemplary aspects, the antigen binding fragment comprises at least part of both HC variable region and LC variable region. In exemplary aspects, the bispecific antigen-binding protein comprises at least one if not both of the LC or HC variable regions of the presently disclosed CLDN6 antibodies and at least one if not both of the LC and HC variable regions of a second antibody specific for a second antigen. In exemplary instances, the bispecific antigen-binding protein comprises an scFV comprising the LC and HC variable regions of the presently disclosed CLDN6 antibodies and the LC and HC variable regions of a second antibody specific for a second antigen.

In exemplary embodiments, the antigen binding protein is bispecific and binds to CLDN6 and a second antigen. In exemplary instances, the second antigen is a cell surface protein expressed by a T-cell. In exemplary aspects, the cell surface protein is a component of the T-cell receptor (TCR), for example, CD3. In exemplary instances, the second antigen is a costimulatory molecule which assists in T-cell activation, e.g., CD40 or 4-1BB (CD137). In alternative exemplary instances, the second antigen is an immune checkpoint molecule (e.g., a protein involved in an immune checkpoint pathway) selected from B7-H3, B7-H4, BTLA, CTLA4, IDO, KIR, LAG3, NOX2, PD-1, TIM3, VISTA, or SIGLEC7. Optionally, the immune checkpoint molecule is PD-1, LAG3, TIM3, or CTLA4.

The anti-CLDN6 antibodies described herein, or antigen binding portions, bispecific molecules, or fusion proteins comprising CLDN6 binding agents may be used for antibody-based therapies of diseases associated with cells expressing CLDN6. For example, the antibodies may be used for treating solid tumor cancer diseases associated with cells expressing CLDN6, such as breast, lung, ovarian, testicular, pancreatic, gastric, gallbladder and urothelial cancer.

In various embodiments, anti-CLDN6 antibodies provided herein may comprise substitutions or modifications of the constant region (i.e. the Fc region), including without limitation, amino acid residue substitutions, mutations and/or modifications, which result in a compound with preferred characteristics including, but not limited to: altered pharmacokinetics, increased serum half-life, increase binding affinity, reduced immunogenicity, increased production, altered Fc ligand binding to an Fc receptor (FcR), enhanced or reduced ADCC, CDC, ADCP, TDCC, altered glycosylation and/or disulfide bonds and modified binding specificity. Antibodies or antibody fragments—with improved Fc effector functions can be generated, for example, through changes in amino acid residues involved in the interaction between the Fc domain and an Fc receptor (e.g., FcyRI, FcyRIIA and B, FCYRIII and FcRn), which may lead to increased cytotoxicity.

A critical step in the activation of cytotoxic cells is the binding of mAbs to FcγRIIIa (CD16A) on immune effector cells, and the strength of this interaction is determined by antibody isotype, the glycosylation pattern of the antibody Fc region and FcγRIIIa polymorphisms. Numerous publications have reported findings that demonstrate the role of FcγR-mediated effector function in antibody-based cancer therapies derived from clinical studies. The study results indicate an association between clinical response (e.g., antibody efficacy) and specific alloforms of activating human FcγRs. Patients that carry the 158F allele have been reported to show diminished clinical responses to certain therapeutic antibodies, including trastuzumab, rituximab, cetuximab, infliximab and ipilimumab and other therapeutic antibodies that utilize ADCC as a major mechanism of action. Antibodies engineered to have improved FcgR binding profiles have been reported to drive superior anti-tumor responses and confer greater clinical benefit.

The discovery of activating and inhibitory FcγRs resulted in translational research efforts focused on designing therapeutic antibodies that were “fit for purpose” based on having FcγR binding activities characterized by an activating/inhibiting (A:I) ratio designed to activate immune effector cells to perform particular functions. Immunotherapy of cancer with monoclonal antibodies (mAb) promotes elimination of tumor cells by a variety of mechanisms including ADCC, ADCP and/or CDC activities. In practice, the therapeutic activity of several approved mAbs depends on the binding of the Fcγ regions to low-affinity Fcγ receptors expressed on effector cells.

Several publications report the successful use of protein engineering strategies to design variant human IgG1 Fc domain (CH regions) with optimized FcgR binding profiles and activating/inhibiting (A:I) ratios suitable to optimize cell-mediated effector functions. In particular efforts have focused on increasing the affinity of the Fc domain for the low affinity receptor FcγIIIa. A number of mutations within the Fc domain have been identified that either directly or indirectly enhance binding of Fc receptors and as a result significantly enhance cellular cytotoxicity (Lazar, G. A. PNAS 103:4005-4010 (2006), Shields, R. L. et al, J. Biol. Chem. 276:6591-6604 (2001) Stewart, R. et al., Protein Engineering Design and Selection 24:671-678 (2011) (Richards, J. O. et al, Mol. Cancer Ther. 7:2517-2575 (2008).

### CLDN6 Binding

The anti-CLDN6 antibodies or antibody fragments thereof provided herein bind to CLDN6 in a non-covalent and reversible manner. In various embodiments, the binding strength of the antigen binding protein to CLDN6 may be described in terms of its affinity, a measure of the strength of interaction between the binding site of the antigen-binding protein and the epitope. In various aspects, the affinities of the antigen-binding proteins are measured or ranked using a flow cytometry- or Fluorescence-Activated Cell Sorting (FACS)-based assay. Flow cytometry-based binding assays are known in the art. See, e.g., Cedeno-Arias et. al., Sci Pharm 79(3): 569-581 (2011); Rathanaswami et. al., Analytical Biochem 373: 52-60 (2008); and Geuijen et. al., J Immunol Methods 302(1-2): 68-77 (2005). Selectivity may be based on the KD exhibited by the antigen binding protein for CLDN6, or a CLDN family member, wherein the KD may be determined by techniques known in the art, e.g., surface plasmon resonance, FACS-based affinity assays.

In various aspects, the relative affinity of a CLDN6 antibody is determined via a FACS-based assay in which different concentrations of a CLDN6 antibody are incubated with cells expressing CLDN6 and the fluorescence emitted (which is a direct measure of antibody-antigen binding) is determined. A curve plotting the fluorescence for each dose or concentration is made. The max value is the lowest concentration at which the fluorescence plateaus or reaches a maximum, which is when binding saturation occurs. Half of the max value is considered an EC₅₀ or an IC₅₀ and the antibody with the lowest EC₅₀/IC₅₀ is considered as having the highest affinity relative to other antibodies tested in the same manner.

In one aspect, the cells are genetically-engineered to overexpress CLDN6. For example, the cells are HEK293T or CHO cells engineered to express CLDN6. In alternative aspects, the cells are established humor tumor cell lines endogenously expressing CLDN6. In various aspects, the cells are cells from a human cell line (e.g., an ovarian cell line, endometrial cell line, germ cell tumor cell line, lung cell line, gastrointestinal (GI) cell line, liver cell line, lung cell line, and the like).

In one embodiment, the anti-CLDN6 antibodies or antibody fragments of the present disclosure selectively bind to CLDN6 relative to CLDN9 and do not bind to Claudin-3 (CLDN3) or to Claudin-4 (CLDN4). In an alternative embodiment, the anti-CLDN6 antibodies or antibody fragments bind to both CLDN6 and CLDN 9 equally (e.g., no preference for either CLDN) and do not bind to Claudin-3 (CLDN3) or to Claudin-4 (CLDN4).

### CLDN6 Internalization and Dose Dependent Cytotoxicity

Preclinical characterization of the safety and antitumor activity of IMAB027-vcMMAE, an ADC comprising the anti-Claudin-6 antibody-drug antibody IMAb027, included studies evaluating: the internalization of IMAB027 in various CLDN6+ human ovarian (OC) and testicular cancer (TC) cell lines; binding characteristics (via FACS) and cell viability and IMAB027-vcMMAE-mediated cytotoxic effects (direct and indirect bystander) assessed in cell cultures by the XTT metabolic assay (Türeci, et al, AACR; Cancer Res 2018; 78 (13Suppl): Abstract #1778).

Türeci, et al. report that IMAB027 ACD binds robustly to, and is internalized by, cell lines expressing CLDN6, and can reduce the viability of CLDN6+ OC and TC cells by up to 100% with EC50 values in the ng/ml order. Additionally, after conjugation, IMAB027-vcMMAE retained IMAB027's ability to induce CLDN6+ cell death via antibody-dependent cellular cytotoxicity and complement-dependent cytotoxicity. Cell lines that did not express CLDN6 were unaffected by IMAB027-vcMMAE in monocultures; however, in cocultures of CLDN6+ and CLDN6-negative cells, IMAB027-vcMMAE exerted bystander effect, resulting in the death of cocultured CLDN6-negative cells in addition to the target-bearing CLDN6+ cells. Therefore, it is known that monoclonal antibodies specific for CLDN6 are capable of mediating the inducible and efficient internalization of CLDN6 and are useful to deliver cytotoxic agent to tumor cells expressing CLDN6.

Based on in vitro assessment of maximum binding capacity, EC50, cell surface internalization and cytotoxicity the disclosed anti-CLDN6 antibodies can be evaluated for suitability for use as an ADC-based targeting antibody for the treatment of cancer. Therefore, the disclosed anti-CLDN6 antibodies are suitable for use as ADC-based targeting antibodies for the development of an internalizing site-specific ADC for use in a method of antibody-based immunotherapies for the treatment of cancer.

The disclosed antibodies specific for CLDN6 are capable of mediating the inducible and efficient internalization of CLDN6, which lead to dose-dependent cytotoxicity when an ADC-conjugated secondary antibody is present. In HEK293 cell line overexpressing CLDN6, the observed EC₅₀ for cell killing ranges from 1.73 nM to 2.19 nM, In cancer cell line NEC8, the EC₅₀ for cell killing ranges from 0.1 nM to 0.2 nM. In cancer cell line OV90, the EC₅₀ for cell killing ranges from 1.08 nM and 2.32 nM.

### CLDN6 ADCC

As a consequence of binding CLDN6 expressed on the surface of a target cell, the disclosed antibodies can mediate target cell killing by one or more mechanisms of action, such as delivery of a cytotoxic agent, or by directing ADCC-, CDC-, or TDCC-mediated lysis. In one embodiment, the target cells are primary or metastatic cancer cells.

In some aspects, the disclosed anti-CLDN6 produced antibodies can be assessed for their ability to mediate killing (e.g., antibody dependent cell mediated cytotoxicity (ADCC), complement dependent cytotoxicity (CDC) T-cell dependent cellular cytotoxicity (TDCC), and/or inhibition of cell proliferation) and/or phagocytosis of cells expressing CLDN6.

The disclosed anti-CLDN6 antibodies are also capable of directing ADCC against target cells expressing CLDN6 either endogenously or by host cells engineered to overexpress human CLDN6. In cancer cell line NEC8, the EC₅₀ for ADCC activity ranges from 0.40 nM to 9.83 nM. In cancer cell line OV90, the EC₅₀ for ADCC activity ranges from 0.3 nM and 0.75 nM.

### Antibody-Based Immunotherapy

The goal of antibody-based immunotherapy using tumor-antigen-targeting antibodies is to eliminate cancer cells without harming normal tissue. Therefore, the efficacy and safety of antibody-based immunotherapies in oncology vary depending in large part on the intended mechanism of action, the relevant effector function of the immune system and the nature of the tumor-specific or tumor-associated target antigen.

Antibodies of the disclosure can also be used to target payloads (e.g., radioisotopes, drugs or toxins) to directly kill tumor cells or can be used synergistically with traditional chemotherapeutic agents, attacking tumors through complementary mechanisms of action that may include anti-tumor immune responses that may have been compromised owing to a chemotherapeutic's cytotoxic side effects on immune effector cells.

Antibody-drug conjugates (ADCs) are a class of highly potent antibody-based cancer therapeutics. ADCs consist of recombinant monoclonal antibodies covalently linked to cytotoxic agents (known as payloads) via synthetic linkers. ADCs combine the specificity of monoclonal antibodies and the potency of small-molecule chemotherapy drugs and facilitate the targeted delivery of highly cytotoxic small molecule drug moieties directly to tumor cells. The targeted nature of ADCs allows for increased drug potency coupled with limited systemic exposure. Together, these features provide ADCs with the desirable characteristics of having fewer side effects and a wider therapeutic window (Peters et al., Biosci Rep, 35 (4): e00225, 2015).

Cell surface antigens suitable for use as ADC targets are characterized by two important properties: (i) high expression level by the target cell and limited or no expression in normal tissue and (ii) efficient internalization in response to antibody binding. CLDN6 is overexpressed in multiple cancers including endometrial, ovarian and testis cancer and lung cancer (NSCLC). There is evidence that a substantial portion of expressed CLDN protein remains associated with the tumorigenic cell surface, thereby allowing for localization and internalization of the disclosed antibodies or ADCs.

As used herein, an antibody that “internalizes” is one that is taken up (along with any cytotoxin) by the cell upon binding to an associated antigen or receptor. For therapeutic applications, internalization will preferably occur in vivo in a subject in need thereof. The number of ADCs internalized may be sufficient to kill an antigen-expressing cell, especially an antigen-expressing cancer stem cell. Depending on the potency of the cytotoxin or ADC as a whole, in some instances, the uptake of a single antibody molecule into the cell is sufficient to kill the target cell to which the antibody binds. For example, certain drugs are so highly potent that the internalization of a few molecules of the toxin conjugated to the antibody is sufficient to kill the tumor cell. Whether an antibody internalizes upon binding to a mammalian cell can be determined by various art-recognized assays including those described in the Examples below.

The generation of antibody-drug conjugates can be accomplished by any technique known to the skilled artisan using any suitable payload drug, synthetic linker and conjugation chemistry. Those skilled in the art will be aware of ADCs and will also be aware that the development of an ADC requires an evaluation of several factors including target antigen biology, specificity of the antibody, cytotoxicity and mechanism of action of the payload drug, the stability and cleavage of the linker, the sites of linker attachment, and the levels of ADC heterogeneity produced by the conjugation chemistry. Heterogeneity, with respect to the number of cytotoxic molecules attached per antibody can result in the production of a drug product containing non-potent species (no drug payload) and species with more than 4 drug moieties (high loading) per antibody that have the potential to be cleared more rapidly and contribute to toxicity. Further, the presence of non-potent species (antibodies with no cytotoxic payload) can decrease potency by competing for binding to the ADC target antigen. Therefore, it is desirable to produce ADC drug products with homogenous mixtures of antibodies characterized by a consistent drug: antibody ratio (DAR).

A majority of the ADC candidates currently under clinical evaluation employ one of the three major classes of drugs as cytotoxic payloads, namely maytansinoids, auristatins, and PBD dimers; but other classes of payloads, such as calicheamicin (for gemtuzumab ozogamicin and inotuzumab ozogamicin), duocarmycin, exatecan or SN-38 are also used (Shim et al., Biomolecules, 10 (3): 360, 2020). Generally speaking, the cytotoxic drugs act either as tubulin inhibitors (auristatins and maytansinoids) or as disruptors of DNA structure, including duocarmycin (DNA alkylation), calicheamicin (DNA double strand cleavage), camptothecin analogues (topoisomerase inhibitor) such as SN-38 and exatecan, or pyrrolobenzodiazepine (PBD) dimers (DNA strand crosslinking) (Shim et al.).

One of the key functions of the linker is to maintain ADC stability in the blood circulation, while allowing toxin release upon internalization by the target cells. Important parameters to be considered during for the identification of a suitable linker include the cleavability of the linker and the details of the conjugation chemistry (i.e., the position and nature of the linkage). Broadly speaking linkers are classified into two broad categories: cleavable and non-cleavable. Cleavable linkers exploit the differences between normal physiologic conditions in the bloodstream and the intracellular conditions present in the cytoplasm of cancer cells (Peters et al., Biosci Rep, 35 (4): e00225, 2015). Changes in the microenvironment after an ADC-antigen complex is internalized, triggers cleavage of the linker and releases the cytotoxic payload, effectively targeting toxicity to cancer cells expressing the target antigen. Broadly speaking there are three types of cleavable linkers: hydrazone, disulfide and peptide linkers. In contrast, non-cleavable linkers depend solely on the process of lysosomal degradation following ADC-antigen internalization. After internalization of the ADC-antigen complex protease enzymes within the lysosome degrade the protein structure of the antibody, leaving a single amino acid (typically a cysteine or a lysine) attached to the linker and the cytotoxic agent that is released into the cytoplasm as the active drug. It is well known that linker chemistry is an important determinant of the specificity, potency, activity and safety of ADCs.

One of skill in the art will recognize that there are many techniques for chemical modification of proteins suitable for use in the conjugation of the linker-payload to a TSA- or TAA-specific antibody. The same person will recognize that different methods of conjugation chemistry will afford different levels of control over the number and site of drug attachment and potentially impact the pharmacokinetics, toxicity and therapeutic window of the anti-CLDN6 ADC that is produced. Antibody-drug conjugates can be prepared by binding the drug to an antibody in accordance with a conventional technique. Techniques for conjugating a therapeutic moiety to antibodies are well known to those of skill in the art, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies ‘84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982).

One of skill in the art will also appreciate that in addition to the conventional conjugation techniques (involving conjugation to surface exposed lysine or cysteine residues present in an antibody either as a consequence of the native amino acid sequence composition) there are numerous methods of site-specific drug conjugation that can be used to prepare anti-CLDN6 specific immunoconjugates. Site-specific conjugation chemistry methods are intended to produce relatively homogenous ADC products without altering the binding affinity of the antibody. Generally speaking, three strategies are mainly used for site-specific conjugation on antibodies: use of engineered cysteines, incorporation of unnatural amino acids and enzymatic conjugations using reaction sites of antibodies that are designed to react specifically to a bacterial enzyme (e.g. transglutaminases, glycotransferases, sortases or formyl glycine generating enzyme) that generate post-translational modifications of proteins in a site-specific manner. Techniques for the site-specific conjugation a therapeutic moiety to antibodies are well known to those of skill in the art and include, but are not limited to the methods disclosed in U.S. Pat. Nos. 7,723,485; 8,937,161; 9,000,130; 9,884,127; 9,717,803; 10,639,291; 10,357,472 U.S. Patent Application Publication Nos: US 2015/0283259; US 2017/0362334; US 2018/0140714; and International Publication Nos.: WO2013/092983; WO2013/092998; WO2014/072482; WO2014/202773; WO2014/202775; WO2015/155753; WO2015/191883; WO2016/102632; WO2017/059158; WO 2018/140590 and WO 2018/185526.

In alternative embodiments, the disclosed anti-CLDN6 antibodies or antibody fragments may interact with effector cells of the immune system, preferably through ADCC, TDCC, CDC, or ADCP (Kubota, T. et al. (2009) Cancer Sci. 100 (9), 1566-1572; Nazarian et al., J. Bio. Scre., 2015, 20(4) 519-527).

The term “immune effector functions” in the context of the present disclosure includes any functions mediated by components of the immune system that result in the inhibition of tumor growth and/or inhibition of tumor development, including inhibition of tumor dissemination and metastasis. Preferably, immune effector functions result in killing of cancer cells. Preferably, the immune effector functions in the context of the present disclosure are antibody-mediated effector functions. Such functions comprise complement dependent cytotoxicity (CDC), antibody-dependent cell-mediated cytotoxicity (ADCC), antibody-dependent cell-mediated phagocytosis (ADCP), induction of apoptosis in the cells carrying the tumor-associated antigen.

Antibody-dependent cell-mediated cytotoxicity (ADCC) describes the cell-killing ability of effector cells, which preferably requires the target cell being marked by an antibody. Effector cells may include B cells, T cells, killer cells, NK cells, macrophages, monocytes, eosinophils, neutrophils, polymorphonuclear cells, granulocytes, mast cells, and/or basophils; more specifically effector cells are T cells or NK cells. In certain aspects, ADCC occurs when antibodies bind to antigens on tumor cells, and the antibody Fc domains engage Fc receptors (FcR) on the surface of immune effector cells. Several families of Fc receptors have been identified, and specific cell populations characteristically express defined Fc receptors. ADCC can be viewed as a mechanism to directly induce a variable degree of immediate tumor destruction that leads to antigen presentation and the induction of tumor-directed T-cell responses. Preferably, in vivo induction of ADCC will lead to tumor-directed T-cell responses and host-derived antibody responses.

Complement-dependent cytotoxicity (CDC) is another cell-killing method that can be directed by antibodies. IgM is the most effective isotype for complement activation, but IgG1 and IgG3 are also both very effective at directing CDC via the classical complement-activation pathway.

Alternatively, the disclosed anti-CLDN6 antibodies provided herein may be utilized in adoptive immunity gene therapy to treat tumors. In one embodiment the antibodies of the disclosure (e.g. ScFv fragments) may be used to generate a chimeric antigen receptor (CAR). A “CAR” is a fused protein made up of an ECD comprising the anti-CLDN antibodies of the disclosure or immunoreactive fragments thereof (e.g., ScFv fragments), a transmembrane domain, and at least one intracellular domain. In one embodiment, T-cells, natural killer cells or dendritic cells that have been genetically engineered to express CARs can be introduced into a subject suffering from cancer in order to stimulate the immune system of the subject to specifically target tumor cells expressing CLDN6.

### Methods of Producing Antibodies

Anti-CLDN6 antibodies or antibody fragments thereof may be made by any method known in the art. For example, a recipient may be immunized with soluble recombinant Claudin-6 (CLDN6) protein or a fragment of a CLDN6 peptide conjugated with a carrier protein thereof. Any suitable method of immunization can be used. Such methods can include adjuvants, other immune stimulants, repeat booster immunizations, and the use of one or more immunization routes.

Any suitable source of human CLDN6 can be used as the immunogen for the generation of the non-human or human anti-CLDN6 antibodies of the compositions and methods disclosed herein.

Different forms of a CLDN6 antigens may be used to elicit an immune response for the identification of a biologically active anti-CLDN6 antibody. Thus, the eliciting CLDN6 antigen may be a single epitope, multiple epitopes, or the entire protein alone or in combination with one or more immunogenicity enhancing agents. In some aspects, the eliciting antigen is an isolated soluble full-length protein, or a soluble protein comprising less than the full-length sequence (e.g., immunizing with a peptide comprising the extracellular domains/loops of CLDN6, ECD1 and/or ECD2 alone or in combination). As used herein, the term “portion” refers to the minimal number of amino acids or nucleic acids, as appropriate, to constitute an immunogenic epitope of the antigen of interest. Any genetic vectors suitable for transformation of the cells of interest may be employed, including, but not limited to adenoviral vectors, plasmids, and non-viral vectors, such as cationic lipids.

It is desirable to prepare monoclonal antibodies (mAbs) from various mammalian hosts, such as mice, rodents, primates, humans, etc. Description of techniques for preparing such monoclonal antibodies may be found in, e.g., Sties et al. (eds.) BASIC AND CLINICAL IMMUNOLOGY (4th ed.) Lance Medical Publication, Los Altos, C A, and references cited therein; Harlow and Lane (1988) ANTIBODIES: A LABORATORY MANUAL CSH Press; Goding (1986) MONOCLONAL ANTIBODIES: PRINCIPLES AND PRACTICE (2nd ed.) Academic Press, New York, NY. Typically, spleen cells from an animal immunized with a desired antigen are immortalized, commonly by fusion with a myeloma cell. See Kohler and Milstein (196) Eur. J. Immunol. 6:511-519. Alternative methods of immortalization include transformation with Epstein Barr Virus, oncogene, or retroviruses, or other methods known in the art. See. e.g., Doyle et al. (eds. 1994 and periodic supplements) CELL AND TISSUE CULTURE: LABORATORY PROEDURES, John Wiley and Sons, New York, NY. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or an antigen binding fragment thereof by screening a DNA library from human B cells according, e.g., to the general protocol outlined by Huse et al. (1989) Science 246:1275-1281. Thus, antibodies may be obtained by a variety of techniques familiar to researchers skilled in the art.

Other suitable techniques involve selection of libraries of antibodies in phage, yeast, virus or similar vector. See e.g., Huse et al. supra; and Ward et al. (1989) Nature 341:544-546. The polypeptides and antibodies disclosed herein may be used with or without modification, including chimeric or humanized antibodies. Frequently, the polypeptides and antibodies will be labeled by joining, either covalently or non-covalently, a substance which provides for a detectable signal. A wide variety of labels and conjugation techniques are known and are reported extensively in both the scientific and patent literatures. Suitable labels include radionuclides, enzymes, substrates, cofactors, inhibitors, fluorescent moieties, chemiluminescent moieties, magnetic particles, and the like. Patents teaching the use of such labels include U.S. Pat. Nos. 3,817,837; 3,850,752; 3,9396,345; 4,277,437; 4,275,149; and 4,366,241. Also, recombinant immunoglobulins may be produced, see Cabilly U.S. Pat. No. 4,816,567; and Queen et al. (1989) Proc. Nat'l Acad. Sci. USA 86:10029-10023; or made in transgenic mice, see Nils Lonberg et al. (1994), Nature 368:856-859; and Mendez et al. (1997) Nature Genetics 15: 146-156; TRANSGENIC ANIMALS AND METHODS OF USE (WO 2012/62118), Medarex, Trianni, Abgenix, Ablexis, OminiAb, Harbour and other technologies.

In some embodiments, the ability of the produced antibody to bind to CLDN6 and/or other related members of the Claudin family can be assessed using standard binding assays, such as surface plasmon resonance (SPR), FoteBio (BLI), Gator (BLI), ELISA, Western Blot, Immunofluorescence, flow cytometric analysis (FACS) or an internalization assay.

The antibody composition prepared from the hybridoma or host cells can be purified using, for example, hydroxylapatite chromatography, gel electrophoresis, dialysis, and affinity chromatography, with affinity chromatography being a typical purification technique. The suitability of protein A as an affinity ligand depends on the species and isotype of any immunoglobulin Fc domain that is present in the antibody. Protein A can be used to purify antibodies that are based on human gamma1, gamma2, or gamma4 heavy chains (see, e.g., Lindmark et al., 1983 J. Immunol. Meth. 62:1-13). Protein G is recommended for all mouse isotypes and for human gamma3 (see, e.g., Guss et al., 1986 EMBO J. 5:1567-1575). A matrix to which an affinity ligand is attached is most often agarose, but other matrices are available. Mechanically stable matrices such as controlled pore glass or poly (styrenedivinyl)benzene allow for faster flow rates and shorter processing times than can be achieved with agarose. Where the antibody comprises a CH3 domain, the Bakerbond ABX™ resin (J. T. Baker, Phillipsburg, N.J.) is useful for purification. Other techniques for protein purification such as fractionation on an ion-exchange column, ethanol precipitation, reverse phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, typically performed at low salt concentrations (e.g., from about 0-0.25M salt).

Also included are nucleic acids that hybridize under low, moderate, and high stringency conditions, as defined herein, to all or a portion (e.g., the portion encoding the variable region) of the nucleotide sequence represented by isolated polynucleotide sequence(s) that encode an antibody or antibody fragment of the present disclosure. The hybridizing portion of the hybridizing nucleic acid is typically at least 15 (e.g., 20, 25, 30 or 50) nucleotides in length. The hybridizing portion of the hybridizing nucleic acid is at least 80%, e.g., at least 90%, at least 95%, or at least 98%, identical to the sequence of a portion or all of a nucleic acid encoding an anti-CLDN6 polypeptide (e.g., a heavy chain or light chain variable region), or its complement. Hybridizing nucleic acids of the type described herein can be used, for example, as a cloning probe, a primer, e.g., a PCR primer, or a diagnostic probe.

### Polynucleotides, Vectors, and Host Cells

Other embodiments encompass isolated polynucleotides that comprise a sequence encoding an anti-CLDN6 antibody or antibody fragment thereof, vectors, and host cells comprising the polynucleotides, and recombinant techniques for production of the antibody. The isolated polynucleotides can encode any desired form of the anti-CLDN6 antibody including, for example, full length monoclonal antibodies, Fab, Fab′, F(ab′)₂, and Fv fragments, diabodies, linear antibodies, single-chain antibody molecules, and multispecific antibodies formed from antibody fragments.

Some embodiments include isolated polynucleotides comprising sequences that encode the heavy chain variable region of an antibody or antibody fragment having the amino acid sequence of SEQ ID NOs: 1, 3, 23, 24, 26, 28 and 30. Some embodiments include isolated polynucleotides comprising sequences that encode the light chain variable region of an antibody or antibody fragment having the amino acid sequence of any of SEQ ID NOs: 2, 4, 25, 27, 29 and 31.

In an embodiment, the isolated polynucleotide sequence(s) encodes an antibody or antibody fragment having a light chain and a heavy chain variable region comprising the amino acid sequences of:
(a) a variable heavy chain sequence comprising SEQ ID NO: 1 and a variable light chain sequence comprising SEQ ID NO: 2;
(b) a variable heavy chain sequence comprising SEQ ID NO: 3 and a variable light chain sequence comprising SEQ ID NO: 4;
(c) a variable heavy chain sequence comprising SEQ ID NO: 23 and a variable light chain sequence comprising SEQ ID NO: 2;
(d) a variable heavy chain sequence comprising SEQ ID NO: 24 and a variable light chain sequence comprising SEQ ID NO: 25;
(e) a variable heavy chain sequence comprising SEQ ID NO: 26 and a variable light chain sequence comprising SEQ ID NO: 27;
(f) a variable heavy chain sequence comprising SEQ ID NO: 28 and a variable light chain sequence comprising SEQ ID NO: 29; and
(g) a variable heavy chain sequence comprising SEQ ID NO: 30 and a variable light chain sequence comprising SEQ ID NO: 31.

In another embodiment, the isolated polynucleotide sequence(s) encodes an antibody or antibody fragment having a light chain and a heavy chain variable region comprising the amino acid sequences of:
(a) a variable heavy chain sequence that is 90%, 95%, or 99% identical to SEQ ID NO: 1 and a variable light chain sequence that is 90%, 95%, or 99% identical to SEQ ID NO: 2; (b) a variable heavy chain sequence that is 90%, 95%, or 99% identical to SEQ ID NO: 3 and a variable light chain sequence that is 90%, 95%, or 99% identical to SEQ ID NO: 4;
(c) a variable heavy chain sequence that is 90%, 95%, or 99% identical to SEQ ID NO: 23 and a variable light chain sequence that is 90%, 95%, or 99% identical to SEQ ID NO: 2;
(d) a variable heavy chain sequence that is 90%, 95%, or 99% identical to SEQ ID NO: 24 and a variable light chain sequence that is 90%, 95%, or 99% identical to SEQ ID NO: 25;
(e) a variable heavy chain sequence that is 90%, 95%, or 99% identical to SEQ ID NO: 26 and a variable light chain sequence that is 90%, 95%, or 99% identical to SEQ ID NO: 27;
(f) a variable heavy chain sequence that is 90%, 95%, or 99% identical to SEQ ID NO: 28 and a variable light chain sequence that is 90%, 95%, or 99% identical to SEQ ID NO: 29; and
(g) a variable heavy chain sequence that is 90%, 95%, or 99% identical to SEQ ID NO: 30 and a variable light chain sequence that is 90%, 95%, or 99% identical to SEQ ID NO: 31.

The polynucleotide(s) that comprise a sequence encoding an anti-CLDN6 antibody or antibody fragment thereof can be fused to one or more regulatory or control sequence, as known in the art, and can be contained in suitable expression vectors or host cell as known in the art. Each of the polynucleotide molecules encoding the heavy or light chain variable domains can be independently fused to a polynucleotide sequence encoding a constant domain, such as a human constant domain, enabling the production of intact antibodies. Alternatively, polynucleotides, or portions thereof, can be fused together, providing a template for production of a single chain antibody.

For recombinant production, a polynucleotide encoding the antibody is inserted into a replicable vector for cloning (amplification of the DNA) or for expression. Many suitable vectors for expressing the recombinant antibody are available. The vector components generally include, but are not limited to, one or more of the following: a signal sequence, an origin of replication, one or more marker genes, an enhancer element, a promoter, and a transcription termination sequence.

The anti-CLDN6 antibodies or antibody fragments thereof can also be produced as fusion polypeptides, in which the antibody or fragment is fused with a heterologous polypeptide, such as a signal sequence or other polypeptide having a specific cleavage site at the amino terminus of the mature protein or polypeptide. The heterologous signal sequence selected is typically one that is recognized and processed (i.e., cleaved by a signal peptidase) by the host cell. For prokaryotic host cells that do not recognize and process the anti-CLDN6 antibody signal sequence, the signal sequence can be substituted by a prokaryotic signal sequence. The signal sequence can be, for example, alkaline phosphatase, penicillinase, lipoprotein, heat-stable enterotoxin II leaders, and the like. For yeast secretion, the native signal sequence can be substituted, for example, with a leader sequence obtained from yeast invertase alpha-factor (including Saccharomyces and Kluyveromyces α-factor leaders), acid phosphatase, C. albicans glucoamylase, or the signal described in WO90/13646. In mammalian cells, mammalian signal sequences as well as viral secretory leaders, for example, the herpes simplex gD signal, can be used. The DNA for such precursor region is ligated in reading frame to DNA encoding the anti-CLDN6 antibody.

Expression and cloning vectors contain a nucleic acid sequence that enables the vector to replicate in one or more selected host cells. Generally, in cloning vectors this sequence is one that enables the vector to replicate independently of the host chromosomal DNA, and includes origins of replication or autonomously replicating sequences. Such sequences are well known for a variety of bacteria, yeast, and viruses. The origin of replication from the plasmid pBR322 is suitable for most Gram-negative bacteria, the 2-ν. plasmid origin is suitable for yeast, and various viral origins (SV40, polyoma, adenovirus, VSV, and BPV) are useful for cloning vectors in mammalian cells. Generally, the origin of replication component is not needed for mammalian expression vectors (the SV40 origin may typically be used only because it contains the early promoter).

Expression and cloning vectors may contain a gene that encodes a selectable marker to facilitate identification of expression. Typical selectable marker genes encode proteins that confer resistance to antibiotics or other toxins, e.g., ampicillin, neomycin, methotrexate, or tetracycline, or alternatively, are complement auxotrophic deficiencies, or in other alternatives supply specific nutrients that are not present in complex media, e.g., the gene encoding D-alanine racemase for Bacilli.

### Compositions and Methods of Treatment

The disclosure also provides compositions including, for example, pharmaceutical compositions that comprise an anti-CLDN6 antibody or antibody fragment thereof for use as a therapeutic drug for the treatment of patients having an epithelial cell-derived primary or metastatic cancer. In a particular embodiment, the compositions described herein are administered to cancer patients to kill tumor cells. For example, the compositions described herein can be used to treat a patient with a solid tumor characterized by the presence of cancer cells expressing or overexpressing CLDN6. In some aspects, the disclosed compositions can be used to treat breast, lung, ovarian, testicular, pancreatic, gastric, gallbladder and urothelial cancer.

In some aspects, the treatment of cancer represents a field where combination strategies are especially desirable since frequently the combined action of two, three, four or even more cancer drugs/therapies generates synergistic effects which are considerably stronger than the impact of a mono-therapeutic approach. The agents and compositions (e.g., pharmaceutical compositions) provided herein may be used alone or in combination with conventional therapeutic regimens such as surgery, irradiation, chemotherapy and/or bone marrow transplantation (autologous, syngeneic, allogeneic or unrelated). The agents and compositions may also be used in combination with one or more of an antineoplastic agent, a chemotherapeutic agent, a growth inhibitory agent, a cytotoxic agent, an immune checkpoint inhibitor, costimulatory molecule, kinase inhibitors, angiogenesis inhibitors, small molecule targeted therapy drugs, and multi-epitope strategies. Thus, in another embodiment, a cancer treatment may be effectively combined with various other drugs.

In one treatment method, pharmaceutical compositions comprising the anti-CLDN6 antibody can further comprise a therapeutic or toxic agent, either conjugated or unconjugated to the anti-CLDN6 antibody or antibody fragment. In a particular embodiment an anti-CLDN6 antibody is used to target an ADC with a cytotoxic payload to tumors expressing and/or overexpressing CLDN6. In an alternative embodiment an anti-CLDN6 antibody is used to target an ADC with a cytotoxic payload to tumors expressing and/or overexpressing CLDN6 and CLDN9.

The disclosed CLDN6 antibodies can be administered either alone or in combination with other compositions that are useful for treating cancer. In one embodiment, the disclosed antibodies can be administered either alone or in combination with other immunotherapeutics including other antibodies useful for treating cancer. For example, in an embodiment the other immunotherapeutic is an antibody against an immune checkpoint molecule selected from the group consisting of human programmed cell death protein 1 (PD-1), PD-L1 and PD-L2, lymphocyte activation gene 3 (LAG3), NKG2A, B7-H3, B7-H4, CTLA-4, GITR, VISTA, CD137, TIGIT and any combination thereof. In an alternative embodiment, the second immunotherapeutic is an antibody to a tumor specific antigen (TSA) or a tumor associated antigen (TAA). Each combination representing a separate embodiment of the disclosure.

The combination of therapeutic agents discussed herein can be administered concurrently as components of a bispecific or multi-specific binding agent or fusion protein or as a single composition in a pharmaceutically acceptable carrier. Alternatively, a combination of therapeutics can be administered concurrently as separate compositions with each agent in a pharmaceutically acceptable carrier. In another embodiment, the combination of therapeutic agents can be administered sequentially.

The pharmaceutical compositions may be formulated with pharmaceutically acceptable carriers or diluents as well as any other known adjuvants and excipients in accordance with conventional techniques such as those disclosed in Remington: The Science and Practice of Pharmacy, 19th Edition, Gennaro, Ed., Mack Publishing Co., Easton, Pa., 1995. In some aspects, the pharmaceutical composition is administered to a subject to treat cancer.

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion). Depending on the route of administration, the active compound, i.e., antibody, bispecific and multispecific molecule, may be coated in a material to protect the compound from the action of acids and other natural conditions that may inactivate the compound.

Typically, compositions for administration by injection are solutions in sterile isotonic aqueous buffer. Where necessary, the pharmaceutical can also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachet indicating the quantity of the active agent. Where the pharmaceutical is to be administered by infusion, it can be dispensed with an infusion bottle containing sterile pharmaceutical grade water or saline. Where the pharmaceutical is administered by injection, an ampoule of sterile water for injection or saline can be provided so that the ingredients can be mixed prior to administration.

A composition of the present disclosure can be administered by a variety of methods known in the art. As will be appreciated by the skilled artisan, the route and/or mode of administration will vary depending upon the desired results. The active compounds can be prepared with carriers that will protect the compound against rapid releases, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Methods for the preparation of such formulations are generally known to those skilled in the art. See, e.g., Sustained and Controlled Release Drug Delivery Systems, J. R. Robinson, ed., Marcel Dekker, Inc., New York, 1978.

In an alternative embodiment, conventional viral and non-viral based gene transfer methods can be used to introduce nucleic acids encoding the antibodies or fragments thereof, as described herein, in mammalian cells or target tissues. Such methods can be used to administer nucleic acids encoding the antibodies to cells in vitro. In some embodiments, the nucleic acids encoding the antibodies or fragments thereof are administered for in vivo or ex vivo gene therapy uses. In other embodiments, gene delivery techniques are used to study the activity of the antibodies in cell-based or animal models. Non-viral vector delivery systems include DNA plasmids, naked nucleic acid, and nucleic acid complexed with a delivery vehicle such as a liposome. Viral vector delivery systems include DNA and RNA viruses, which have either episomal or integrated genomes after delivery to the cell. Such methods are well known in the art.

Methods of non-viral delivery of nucleic acids encoding engineered polypeptides of the disclosure include lipofection, microinjection, biolistics, virosomes, liposomes, immunoliposomes, polycation or lipid: nucleic acid conjugates, naked DNA, artificial virions, and agent-enhanced uptake of DNA. Lipofection methods and lipofection reagents are well known in the art (e.g., Transfectam™ and Lipofectin™). Cationic and neutral lipids that are suitable for efficient receptor-recognition lipofection of polynucleotides include those of Felgner, WO 91/17424, WO 91/16024. Delivery can be to cells (ex vivo administration) or target tissues (in vivo administration). The preparation of lipid: nucleic acid complexes, including targeted liposomes such as immunolipid complexes, is well known to one of skill in the art.

The use of RNA or DNA viral based systems for the delivery of nucleic acids encoding the antibodies described herein take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of polypeptides of the disclosure could include retroviral, lentivirus, adenoviral, adeno-associated and herpes simplex virus vectors for gene transfer. Viral vectors are currently the most efficient and versatile method of gene transfer in target cells and tissues. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

Dosage levels of the active ingredients in the pharmaceutical compositions may be varied so as to obtain an amount of the active ingredient which is effective to achieve the desired therapeutic response for a particular subject, composition, and mode of administration, without being toxic to the subject. The selected dosage level will depend upon a variety of pharmacokinetic factors including the activity of the particular compositions of the present disclosure employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

The pharmaceutical compositions described herein may be administered in effective amounts. An “effective amount” refers to the amount which achieves a desired reaction or the desired effect alone or together with further doses. In the case of treatment of a particular disease or of a particular condition, the desired reaction preferably relates to inhibition of the course of the disease. This comprises slowing down the progress of the disease and, in particular, interrupting or reversing the progress of the disease.

The broad scope of this disclosure is best understood with reference to the following examples, which are not intended to limit the disclosures to the specific embodiments. The specific embodiments described herein are offered by way of example only, and the disclosure is to be limited by the terms of the appended claims, along with the full scope of the equivalents to which such claims are entitled.

### EXAMPLES

### General Methods

Methods for protein purification including immunoprecipitation, chromatography, and electrophoresis are described. See, e.g., Coligan et al. (2000) Current Protocols in Protein Science, Vol. 1, John Wiley and Sons, Inc., New York. Chemical analysis, chemical modification, post-translational modification, production of fusion proteins, and glycosylation of proteins are described. See, e.g., Coligan et al. (2000) Current Protocols in Protein Science, Vol. 2, John Wiley and Sons, Inc., New York; Ausubel et al. (2001) Current Protocols in Molecular Biology, Vol. 3, John Wiley and Sons, Inc., NY, N. Y., pp. 16.0.5-16.22.17; Sigma-Aldrich, Co. (2001) Products for Life Science Research, St. Louis, Mo.; pp. 45-89; Amersham Pharmacia Biotech (2001) BioDirectory, Piscataway, N.J., pp. 384-391. Production, purification, and fragmentation of polyclonal and monoclonal antibodies are described. Coligan et al. (2001) Current Protocols in Immunology, Vol. 1, John Wiley and Sons, Inc., New York; Harlow and Lane (1999) Using Antibodies, Cold Spring Harbor Laboratory Press, Cold Spring Harbor, N.Y.; Harlow and Lane, supra.

Hybridoma or cell culture supernatant containing an anti-Claudin-6 antibody was purified via HiTrap protein G column (GE, cat. No. 17040401) according to the manufacturer's procedures. Briefly, supernatant was equilibrated with DPBS (Gibco, cat. No. 14190-136) for 5 CV and loaded via syringe/infusion pump (Legato 200, KDS) at ambient temperature and 3 minute residence time. The column was washed with 5 CV of DPBS and elution was performed with 4 CV of pH 2.8 elution buffer (Fisher Scientific, cat. No. PI21004). Elution was fractionated, and fractions were neutralized with 1M Tris-HCL, pH 8.5 (Fisher Scientific, cat No. 50-843-270) and assayed by A280 (DropSense96, Trinean). Peak fractions were pooled, and buffer exchanged into DPBS. Centrifugal filters (EMD Millipore, cat. No. UFC803024) were equilibrated in DPBS at 4,000×g for 2 mins. Purified sample was loaded. DPBS was added and the sample was spun at 4,000×g for 5-10 minute spins until total DPBS volume reached ≥6 DV. The final pool was analyzed by A280.

Standard methods in molecular biology are described. See, e.g., Maniatis et al. (1982) Molecular Cloning, A Laboratory Manual, Cold Spring Harbor Laboratory Press, Cold Spring Harbor, N.Y.; Sambrook and Russell (2001) Molecular Cloning, 3rd ed., Cold Spring Harbor Laboratory Press, Cold Spring Harbor, N.Y.; Wu (1993) Recombinant DNA, Vol. 217, Academic Press, San Diego, Calif. Standard methods also appear in Ausbel et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Stable cell lines expressing human Claudin-6, Claudin-9, Claudin-3, or Claudin-4 were generated by transfecting a selected host cell (i.e., CHO-K1 or HEK293) with pcDNA3.1-based plasmids expressing Homo sapiens Claudin proteins (reference protein sequences in Table 3) using electroporation- or lipid-based transfection. Geneticin or Puromycin was used to select the integrated cells. After 7-10 days of antibiotic selection, stable clones were isolated by FACS or serial dilution using a labelled antibody. After expansion, the stable clones were further confirmed for Claudin protein expression by flow cytometry. Mouse and cynomolgus Claudin-6 (reference sequence in Table 3) were respectively transiently expressed in HEK293T cells using lipid-based transfection.

The NEC8\CLDN6 knockout cell line was generated using CRISPR-Cas9 system. Briefly, a sgRNA targeting CLDN6 Exon 2 was used as ribonucleoprotein complex to transfect NEC8 cells via electroporation. Knockout cell pools were obtained by a sorter and verified by NGS. The KO cell pools were further confirmed by flow cytometry.

TABLE 3
Claudin Protein Sequences

| SEQ ID NO: | NCBI Ref. Seq | Amino Acid Sequence |
|---|---|---|
| SEQ ID NO: | NP_067018.2 | MASAGMQILGVVLTLLGWVNGLVSCALPMW |
| 17 | Human CLDN | KVTAFIGNSIVVAQVVWEGLWMSCVVQSTGQ |
| | 6 | MQCKVYDSLLALPQDLQAARALCVIALLVAL |
| | | FGLLVYLAGAKCTTCVEEKDSKARLVLTSGIV |
| | | FVISGVLTLIPVCWTAHAIIRDFYNPLVAEAQK |
| | | RELGASLYLGWAASGLLLLGGGLLCCTCPSGG |
| | | SQGPSHYMARYSTSAPAISRGPSEYPTKNYV |
| SEQ ID NO: | XP_005591080.1 | MASAGMQILGVVLTLLGWVNGLVSCALPMW |
| 18 | Cyno CLDN 6 | KVTAFIGNSIVVAQVVWEGLWMSCVVQSTGQ |
| | | MQCKVYDSLLALPQDLQAARALCVIALLVAL |
| | | FGLLVYLAGAKCTTCVEEKDSKARLVLTSGIV |
| | | FVISGVLTLIPVCWTAHAIIRDFYNPLVAEAQK |
| | | RELGASLYLGWAASGLLLLGGGLLCCTCPSGG |
| | | SRGPSHYMARYSTSAPAISRGPSEYPTKNYV |
| SEQ ID NO: | NP_061247.1 | MASTGLQILGIVLTLLGWVNALVSCALPMWK |
| 19 | Mouse CLDN | VTAFIGNSIVVAQMVWEGLWMSCVVQSTGQ |
| | 6 | MQCKVYDSLLALPQDLQAARALCVVTLLIVLL |
| | | GLLVYLAGAKCTTCVEDRNSKSRLVLISGIIFVI |
| | | SGVLTLIPVCWTAHSIIQDFYNPLVADAQKREL |
| | | GASLYLGWAASGLLLLGGGLLCCACSSGGTQ |
| | | GPRHYMACYSTSVPHSRGPSEYPTKNYV |
| SEQ ID NO: | NP_066192.1 | MASTGLELLGMTLAVLGWLGTLVSCALPLWK |
| 20 | Human CLDN | VTAFIGNSIVVAQVVWEGLWMSCVVQSTGQM |
| | 9 | QCKVYDSLLALPQDLQAARALCVIALLLALLG |
| | | LLVAITGAQCTTCVEDEGAKARIVLTAGVILLL |
| | | AGILVLIPVCWTAHAIIQDFYNPLVAEALKREL |
| | | GASLYLGWAAAALLMLGGGLLCCTCPPPQVE |
| | | RPRGPRLGYSIPSRSGASGLDKRDYV |
| SEQ ID NO: | NP_001296.1 | MASMGLQVMGIALAVLGWLAVMLCCALPM |
| 21 | Human CLDN | WRVTAFIGSNIVTSQTIWEGLWMNCVVQSTG |
| | 4 | QMQCKVYDSLLALPQDLQAARALVIISIIVAAL |
| | | GVLLSVVGGKCTNCLEDESAKAKTMIVAGVV |
| | | FLLAGLMVIVPVSWTAHNIIQDFYNPLVASGQ |
| | | KREMGASLYVGWAASGLLLLGGGLLCCNCPP |
| | | RTDKPYSAKYSAARSAAASNYV |
| SEQ ID NO: | NP_001297.1 | MSMGLEITGTALAVLGWLGTIVCCALPMWRV |
| 22 | Human CLDN | SAFIGSNIITSQNIWEGLWMNCVVQSTGQMQC |
| | 3 | KVYDSLLALPQDLQAARALIVVAILLAAFGLL |
| | | VALVGAQCTNCVQDDTAKAKITIVAGVLFLLA |
| | | ALLTLVPVSWSANTIIRDFYNPVVPEAQKREM |
| | | GAGLYVGWAAAALQLLGGALLCCSCPPREKK |
| | | YTATKVVYSAPRSTGPGASLGTGYDRKDYV |

The sequences for the heavy and light chain variable regions for hybridoma clones were determined as described below. Total RNA was extracted from 1-2×10⁶ hybridoma cells using the RNeasy Plus Mini Kit from Qiagen (Germantown, MD, USA). CDNA was generated by performing 5′ RACE reactions using the SMARTer RACE 5′/3′ Kit from Takara (Mountainview, CA, USA). PCR was performed using the Q5 High-Fidelity DNA Polymerase from NEB (Ipswich, MA, USA) to amplify the variable regions from the heavy and light chains using the Takara Universal Primer Mix in combination with gene specific primers for the 3′ mouse constant region of the appropriate immunoglobulin. The amplified variable regions for the heavy and light chains were run on 2% agarose gels, the appropriate bands excised and then gel purified using the Mini Elute Gel Extraction Kit from Qiagen. The purified PCR products were cloned using the Zero Blunt PCR Cloning Kit from Invitrogen (Carlsbad, CA, USA), transformed into Stellar Competent E. Coli cells from Takara and plated onto LB Agar+50 μg/ml kanamycin plates. Direct colony Sanger sequencing was performed by GeneWiz (South Plainfield, NJ, USA). The resulting nucleotide sequences were analyzed using IMGT V-QUEST to identify productive rearrangements and analyze translated protein sequences. CDR determination was based on Kabat numbering.

Selected VH or VL chains were PCR amplified and cloned into a pcDNA3.4-based expression vector, which harbors the constant region from human IgG1 (Uniprot P01857) or human Kappa light chain (UniProt P01834). Paired heavy chain- and light chain-expressing plasmids were transfected into Expi293 cells (Thermo Fisher Scientific) following provider's Expi293 expression system protocol. Five days after transfection culture supernatants were collected by centrifugation. Recombinant antibodies were purified by 1-step affinity purification using Protein A column and buffer exchanged to PBS pH 7.2.

Methods for flow cytometry, including fluorescence activated cell sorting detection systems (FACS®), are available. See, e.g., Owens et al. (1994) Flow Cytometry Principles for Clinical Laboratory Practice, John Wiley and Sons, Hoboken, N.J.; Givan (2001) Flow Cytometry, 2nd ed.; Wiley-Liss, Hoboken, N.J.; Shapiro (2003) Practical Flow Cytometry, John Wiley and Sons, Hoboken, N.J. Fluorescent reagents suitable for modifying nucleic acids, including nucleic acid primers and probes, polypeptides, and antibodies, for use, e.g., as diagnostic reagents, are available. Molecular Probes (2003) Catalogue, Molecular Probes, Inc., Eugene, Oreg.; Sigma-Aldrich (2003) Catalogue, St. Louis, Mo.

Standard techniques for characterizing ligand/receptor interactions are available. See, e.g., Coligan et al. (2001) Current Protocols in Immunology, Vol. 4, John Wiley, Inc., New York. Standard methods of antibody functional characterization appropriate for the characterization of antibodies with particular mechanisms of action are also well known to those of skill in the art.

An in-house anti-CLDN6 antibody based on the anti-CLDN6 antibody (64A) referred to herein as “NR.N6.PC1” (PC1), was prepared based on the publicly available information published in WO2012/156018 (VH SEQ ID NO: 36 and VL SEQ ID NO: 35 therein). The PC1 antibody was used to confirm Claudin-6 expression by the transfected cells and tumor cell lines used in the examples and to establish the binding and functional assays used to evaluate and characterize the anti-CLDN6 specific antibodies disclosed herein. A second in-house CLDN6/9 reactive antibody (hsC27.22), referred to herein as “NR.N6.PC2” (PC2), was prepared based on publicly available information published in WO2015/069794 (VH SEQ ID NO: 67 and VL SEQ ID NO: 65 therein).

Software packages and databases for determining, e.g., antigenic fragments, leader sequences, protein folding, functional domains, CDR annotation, glycosylation sites, and sequence alignments, are available.

### Example 1: Generation of Anti-CLDN-6 Antibodies

Fully human anti-human CLDN6 antibodies were generated by immunizing human Ig transgenic mice, Trianni mice that express human antibody VH and VL genes (see, e.g., WO 2013/063391, TRIANNI® mice).

Immunization-TRIANNI mice described above were immunized by injection with the immunogens, which including the DNA containing the human Claudin-6 gene and CHO cells stably transfected with the human Claudin-6 gene. The TRIANI mice were immunized with the DNA via tail vein injection. The CHO cells transfected with the human Claudin-6 via intraperitoneally (IP), subcutaneously (SC), based on tail or footpad injections.

The immune response was monitored by retroorbital bleeds. The plasma was screened by flow cytometry (FACS) or Imaging (as described below). Mice with sufficient anti-Claudin-6 titers were used for fusions. Mice were boosted intraperitoneally, at the base of the tail or intravenously with the immunogen before sacrifice and removal of the spleen and lymph nodes.

Selection of mice producing anti-Claudin-6 Antibodies—to select mice producing antibodies that bound Claudin-6, sera from immunized mice were screened by FACS or imaging for binding to cells expressing Claudin-6 protein (CHO transfected with the Claudin-6 gene) not the control cells that do not express Claudin-6 (CHO cells).

For FACS, briefly, Claudin-6-CHO cells or parental CHO cells were incubated with dilutions of serum from immunized mice for 2 hours at 4° C. Cells were fixed with 2% PFA (Alfa Aesar, catalog number: J61899) for 15 minutes at 4° C. and then washed. Specific antibody binding was detected with Alexa 647 labeled goat anti-mouse IgG antibody (ThemoFisher Scientific, catalog number: A-21235) after one-hour incubation at 4° C. Flow cytometric analyses were performed on a flow cytometry instrument (Intellicyte, IQue plus, Sartorius).

In addition, mice serum was tested by imaging. Briefly, Claudin-6-CHO cells were incubated with dilutions of serum from immunized mice. Cells were washed, fixed with paraformaldehyde, washed, specific antibody binding was detected with secondary Alexa488 goat anti-mouse antibody and Hoechst (Invitrogen). Plates were scanned and analyzed on an imaging machine (Cytation 5, Biotek).

Generation of Hybridomas Producing antibodies to CLDN6—to generate hybridomas producing human antibodies of the disclosure, splenocytes and lymph node cells were isolated from an immunized mouse and fused to an appropriate immortalized cell line, such as a mouse myeloma cell line. The resulting hybridomas were screened for the production of antigen-specific antibodies. For example, single cell suspensions of splenocytes, lymph node cells from immunized mice were fused to equal number of Sp2/0 non-secreting mouse IgG myeloma cells (ATCC, CRL 1581) by electrofusion. Cells were plated in flat bottom 96-well tissue culture plates, followed by about one week of incubation in selection medium (HAT medium), then switched to hybridoma culture media. Approximately 10-14 days after cell plating, supernatants from individual wells were screened by Imaging or FACS as described above. The antibody secreting-hybridomas were transferred to 24-well plates, screened again, and if still positive for anti-Claudin-6, the positive hybridomas were subcloned by sorting using a single cell sorter. The subclones were screened again by Imaging or FACS as described above. The stable subclones were then cultured in vitro to generate small amounts of antibodies for purification and characterization.

### Example 2: Binding Specificity of Anti-CLDN6 Antibodies

The binding specificity of the disclosed anti-Claudin-6 antibodies, NR.N6.Ab1 and NR.N6.Ab2), were assessed by FACS using a Claudin-6 transfected cell line Claudin-6-CHO-K1 (GenScript, Item #U3288DL180_3) and parental CHO cell (CHO-K1, ATCC, CCL-61). Briefly, Claudin-6-CHO-K1 cells verse parental CHO-K1 cells were incubated with anti-CLDN6 antibodies for 2 hours at 4° C. Cells were fixed with 2% PFA (Alfa Aesar, cat #: J61899) for 15 minutes at 4° C. and then washed. Specific antibody binding was detected with Alexa 647 labeled goat anti human IgG antibody (ThermoFisher Scientific, cat #: A21445) after one-hour incubation at 4° C. Flow cytometric analyses were performed on a flow cytometry instrument (Intellicyte, IQue plus, Sartorius).

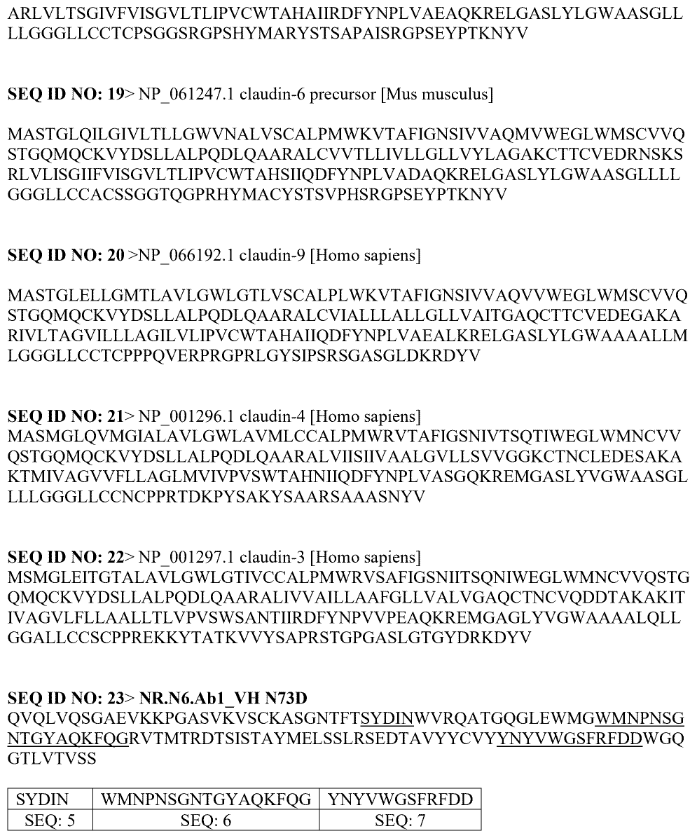

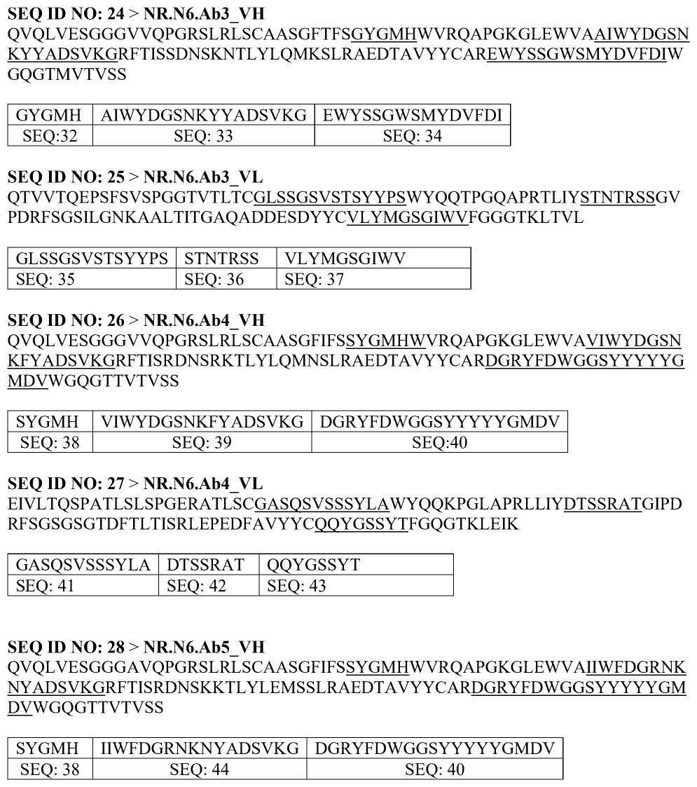

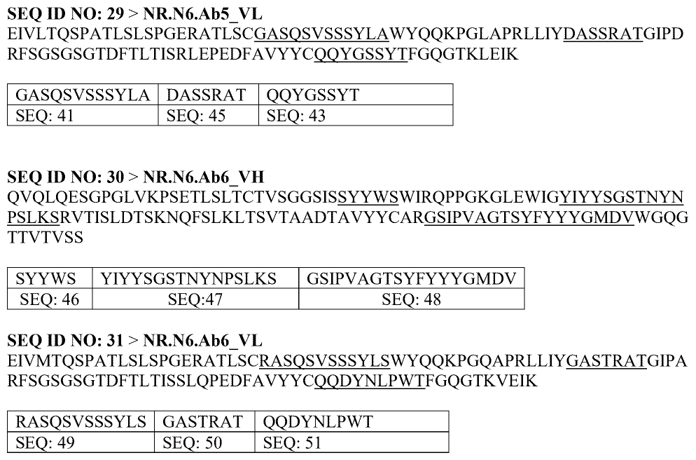

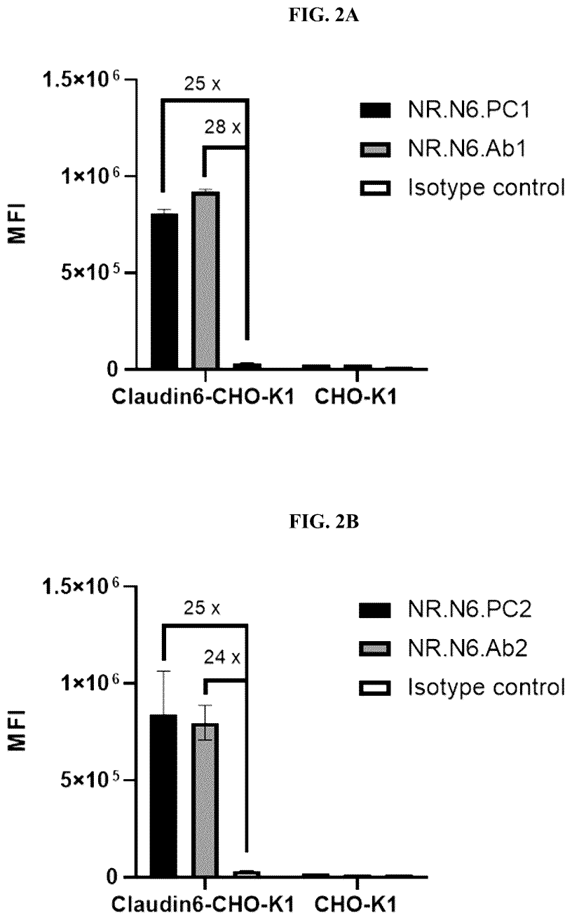

FIGS. 2A and 2B showed that the disclosed anti-Claudin-6 antibodies, NR.N6.Ab1 and NR.N6.Ab2, bound to Claudin-6-CHO-K1 transfected cells (GenScript, Item #U3288DL180_3) with 28-fold and 24-fold MFI, respectively, compared to the isotype control antibody staining at 5 μg/ml. The control antibodies NR.N6.PCI and NR.N6.PC2 bound to Claudin-6-CHO-KI with 25-fold MFI compared to the isotype control antibody. All the antibodies did not bind to the parental CHO-K1 cells (FIGS. 2A and 2B).

The binding specificity of the disclosed anti-Claudin-6 antibodies were further assessed for binding to Claudin-9 by FACS. Briefly, Claudin-9-HEK293 cells (GenScript, Item #U3288DL180_4) were incubated with recombinant Claudin-6 antibodies for 2 hours at 4° C. Cells were fixed with 2% PFA (Alfa Aesar, cat #: J61899) for 15 minutes at 4° C. and then washed. Specific antibody binding was detected with secondary antibody goat-anti-human IgG conjugated with Alexa Fluor 647 (ThermoFisher Scientific, cat #: A21445) after one-hour incubation at 4° C. Flow cytometric analyses were performed on a flow cytometry instrument (Intellicyte, IQue plus, Sartorius).

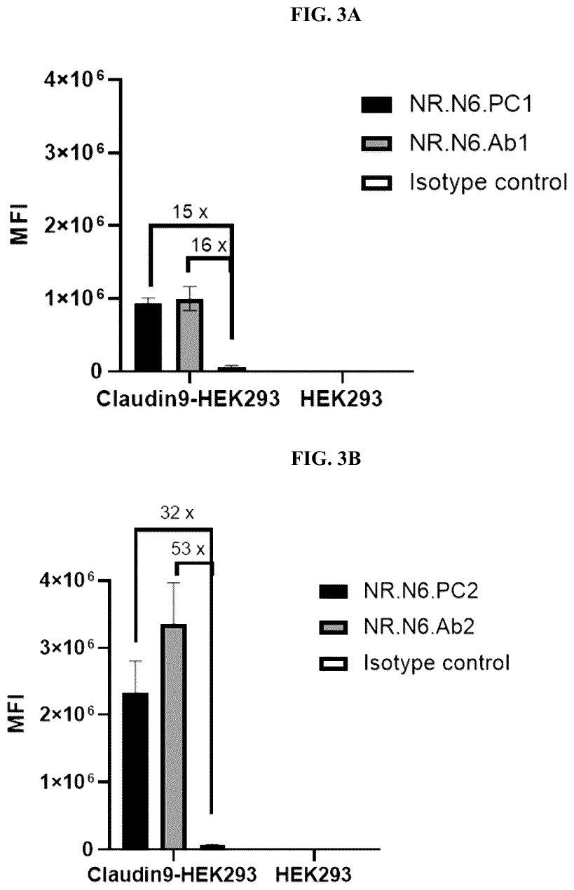

FIGS. 3A and 3B showed the binding activities of the disclosed Claudin-6 antibodies, NR.N6.Ab1 and NR.N6.Ab2 along with the positive controls, NR.N6.PC1 and NR.N6.PC2, to Claudin-9-HEK293 cells versus HEK293 parental cells by FACS (antibody at the concentration of 5 μg/ml). NR.N6.Ab1 bound to Claudin-9-HEK293 cells with 16-fold MFI compared to the isotype control; NR.N6.Ab2 bound to Claudin-9-HEK293 cells with 53-fold MFI compared to the isotype control. The control antibodies NR.N6.PC1 and NR.N6.PC2 bound to human Claudin-9-HEK293 cells with 15-fold and 32-fold MFI higher than the isotype control antibody, respectively. The binding patten of NR.N6.Ab1 was similar to NR.N6.PC1, and the biding patten of NR.N6.Ab2 was similar to NR.N6.PC2. All the tested antibodies did not bind to the parental HEK293 cells (FIGS. 3A and 3B). Previous research showed that the amino acid sequence of Claudin-6 is highly homologues with Claudin-9, 3, and 4 in extracellular (ECL) loop 1 (ECL-1) and loop 2 (ECL-2) (see Table 4 and 5 summarizing % identity between amino acid sequences of the ECL1 and EC2 loops of human CLDN 6, 9, 3 and 4) (Biochemical et Biophysica Acta 1778 (2008) 631-645). Therefore, it is important to evaluate the ability of anti-Claudin-6 antibodies to bind to cells expressing these Claudin family members.

TABLE 4
% Identity of ECL1 (53 aa) of human CLDN6 vs. 9, 3, and 4

| | CLDN6 | CLDN9 | CLDN4 | CLDN3 |
|---|---|---|---|---|
| CLDN6 | | 98.1% | 84.9% | 81.1% |
| CLDN9 | 98.1% | | 83.0% | 79.2% |
| CLDN4 | 84.9% | 83.0% | | 94.3% |
| CLDN3 | 81.1% | 79.2% | 94.3% | |

TABLE 5
% Identity of ECL2 (23 aa) of human CLDN6 vs. 9, 3, and 4

| | CLDN6 | CLDN9 | CLDN4 | CLDN3 |
|---|---|---|---|---|
| CLDN6 | | 91.3% | 78.3% | 73.9% |
| CLDN9 | 91.3% | | 78.3% | 65.2% |
| CLDN4 | 78.3% | 78.3% | | 65.2% |
| CLDN3 | 73.9% | 65.2% | 65.2% | |

To further evaluate the binding characteristics of the disclosed anti-CLDN6 antibodies, NR.N6.Ab1 and NR.N6.Ab2 (purified from hybridoma supernatants) and the two in-house positive controls NR.N6.PC1 and NR.N6.PC2 were tested for binding to Claudin-3-CHO-K1 and Claudin-4-CHO-K1 cells. The binding was evaluated by FACS as described above using anti-claudin 3 (R&D, cat #MAB4620) and anti-Claudin-4 (R&D, cat #MAB4219) antibodies which recognize the native epitopes as Claudin-3 and 4 positive control antibodies.

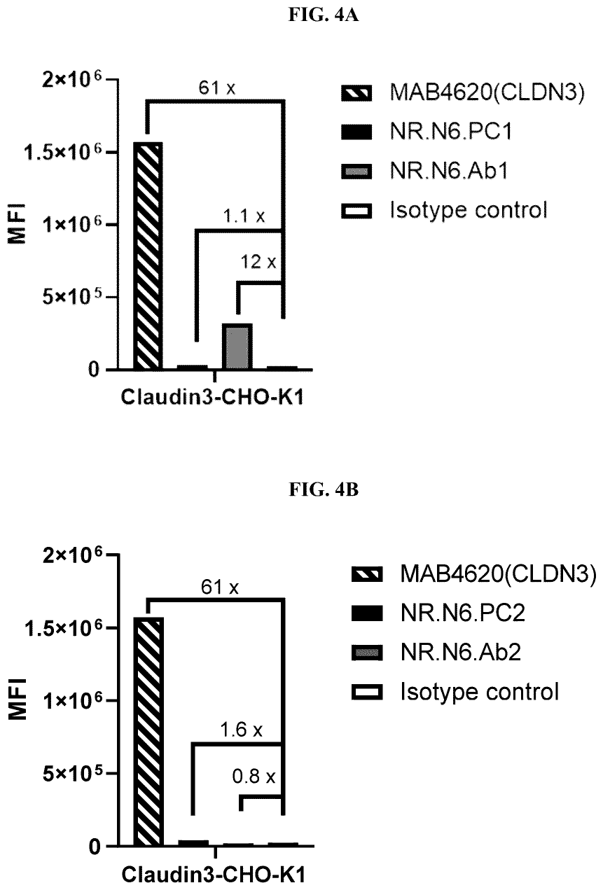

FIGS. 4A and 4B showed that NR.N6.Ab1 bound to Claudin-3-CHO-K1 transfected cells with 12-fold higher MFI than the isotype control. In comparison, the anti-claudin 3 control antibody MAB4620 bound to Claudin-3-CHO-K1 cells with 61-fold higher MFI than the isotype control at the concentration of 5 μg/ml. This observation suggests that NR.N6.Ab1 could be characterized as selective for CLDN3 however NR.N6.Ab1 binding was not observed in a follow-up FACS analysis using MCF7 cells endogenously expressing human CLDN3. The discrepancy may be attributed to a conformational difference in CLDN6 expression by CHO-K1 transfected cells compared to endogenous expression by a human cell. The other anti-Claudin-6 antibody, NR.N6. Ab2, did not bind to Claudin-3 transfected CHO-K1 cells. The two positive control antibodies, NR.N6.PC1 and NR.N6.PC2, also did not bind to Claudin-3-CHO-K1 cells.

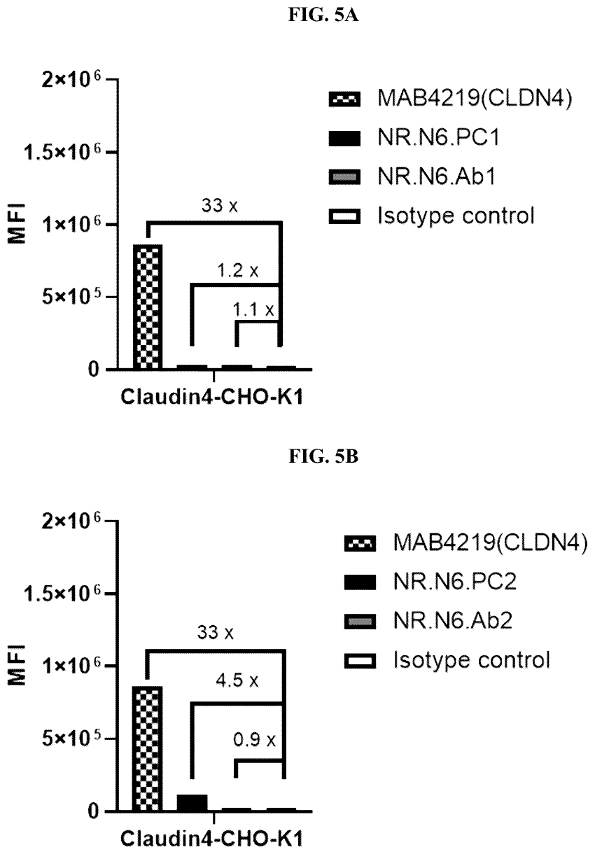

FIGS. 5A and 5B showed that NR.N6.Ab1 and NR.N6.Ab2 did not bind to Claudin-4-CHO-K1 cells. The positive control anti-Claudin-4 antibody MAB4219 bound to Claudin-4-CHO-K1 with 33-fold higher MFI than the isotype control. NR.N6. PC1 did not bind to Claudin-4-CHO-K1 cells while NR.N6.PC2 bound to Claudin-4-CHO-K1 cells with 4.5-fold higher MFI than the isotype control.

Previous research has established that NEC8 (a testicular germ cell tumor cell line) highly expresses endogenous human Claudin-6 and that OV90 (ovarian cancer cell line) expresses lower level of Claudin-6. To determine whether the disclosed anti-Claudin-6 antibodies, NR.N6.Ab1 and NR.N6.Ab2, can bind to CLDN6 expressed on NEC8 and OV90 cells, these two antibodies, along with the two positive control antibodies, NR.N6.PC1 and NR.N6.PC2, were assessed by FACS. Briefly, NEC8 and OV90 cells were incubated with the Claudin-6 recombinant antibodies NR.N6.Ab1, NR.N6.Ab2, NR.N6.PC1 and NR.N6.PC2 for 2 hours at 4° C. Cells were fixed with 2% PFA (Alfa Aesar, catalog number: J61899) for 15 minutes at 4° C. and then washed. Specific antibody binding was detected with secondary antibody goat-anti-human IgG conjugated with Alexa Fluor 647 (Thermo Fisher Scientific, catalog number: A21445) after one-hour incubation at 4° C. Flow cytometric analyses were performed on a flow cytometry instrument (Intellicyte, IQue plus, Sartorius).

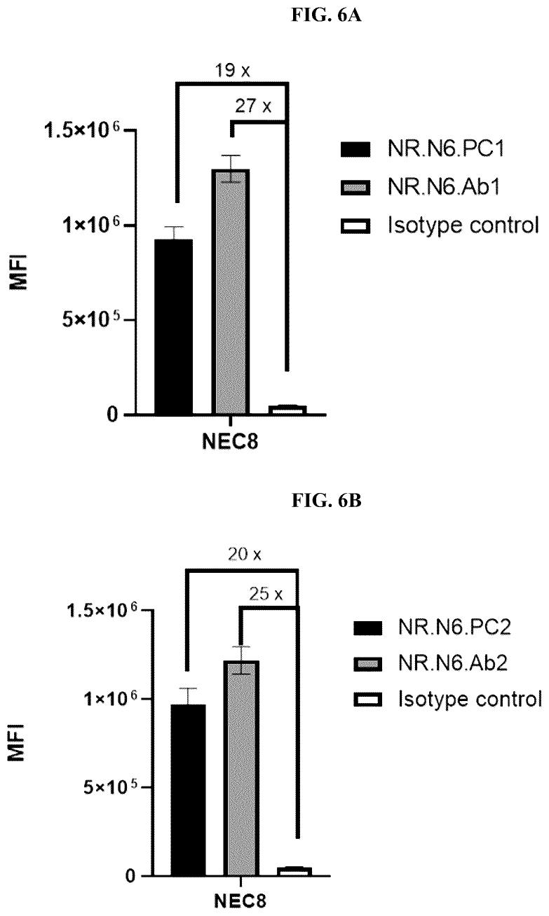

The results from FIGS. 6A and 6B indicated that the disclosed anti-Claudin-6 antibodies NR.N6.Ab1 and NR.N6.Ab2 were able to bind to NEC8 cells with 27-fold and 25-fold higher MFI compared to the isotype control, respectively. The positive control antibodies NR.N6.PC1 and NR.N6.PC2 bound to NEC8 cells with 19-fold and 20-fold binding activities, respectively, compared to the isotype control.

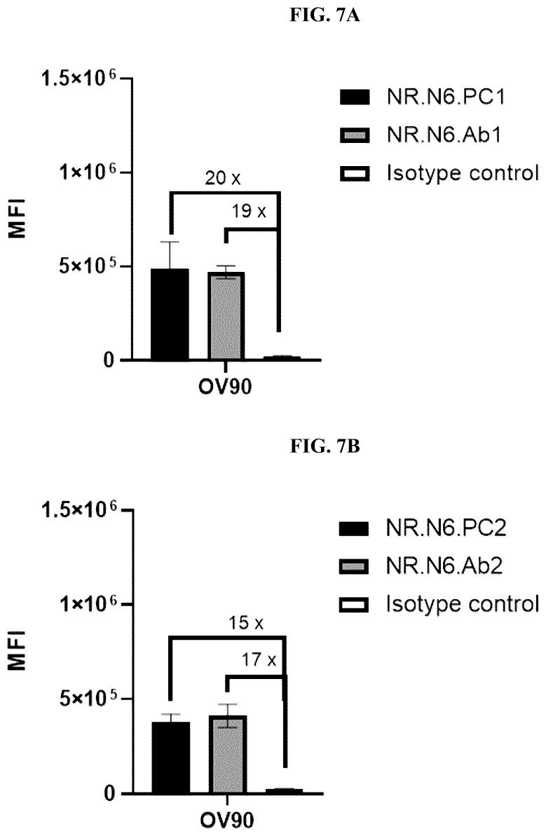

FIGS. 7A and 7B showed that the disclosed anti-Claudin-6 antibodies, NR.N6.Ab1 and NR.N6.Ab2, bound to OV90 cells with 19-fold and 17-fold higher MFI than the isotype control antibody, respectively. The positive control antibodies, NR.N6.PC1 and NR.N6.PC2, bound to OV90 cells with 20-fold and 15-fold higher MFI than the isotype control, respectively.

The disclosed anti-Claudin-6 antibodies, NR.N6.Ab1 and NR.N6.Ab2 (purified from hybridoma), and the two positive control antibodies, NR.N6.PC1 and NR.N6.PC2, were also assessed for binding to MCF7 cell line (an endogenous cell line that is known to express Claudin-3 and 4, WO2019/056023) by FACS using anti-Claudin-3 (R&D, MAB4620) and anti-Claudin-4 (R&D, MAB4219) antibodies as positive control antibodies.

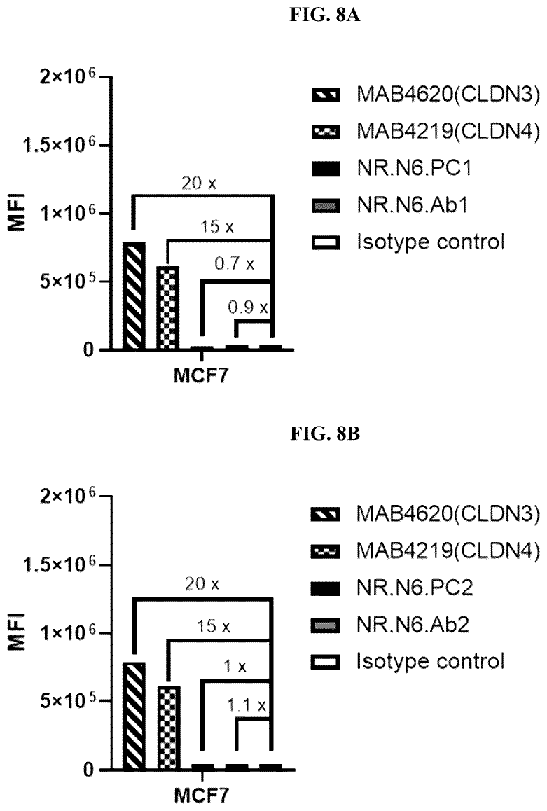

FIGS. 8A and 8B showed that the disclosed anti-Claudin-6 antibodies, NR.N6.Ab1 and NR.N6.Ab2, did not bind to MCF7 cells while the anti-Claudin-3 (MAB4620) and anti-Claudin-4 (MAB4219) antibodies bound to Claudin-3 and Claudin-4 with 20-fold and 15-fold higher MFI than the isotype control, respectively. The control antibodies NR.N6.PC1 and NR.N6.PC2 also did not bind to MCF7 cells.

In order to validate 3 tumor cell lines, NEC8, OV90 and MCF7, for their expression level of Claudin-9, these 3 cell lines along with Claudin-9-HEK293 cell line were tested by FACS using an anti-Claudin-9 polyclonal antibody specific for an intracellular C-terminal epitope (Invitrogen, cat #PA5-67431) as a positive control, via the FACS protocol as described above.

FIG. 9 showed that the anti-Claudin-9 positive control antibody did not bind to NEC8 and OV90 cell lines and had very low binding signal on MCF7 cell line compared to the isotype control antibody whereas the anti-Claudin-9 antibody strongly bound to Claudin-9-HEK293 cells (13 folds higher MFI compared to the isotype control). These results suggest that the human cell lines, NEC8, OV90 and MCF7, do not express Claudin-9.

Overall, the results of the FACS binding experiments described above indicate that the disclosed antibody NR.N6.Ab1 binds strongly to CLDN6 and weakly to Claudin-9 compared to the isotype control. NR.N6.Ab1 binds to CLDN3 with a detectable but weak binding signal (20% of the positive control) whereas the CLDN3 positive control antibody had much higher binding signal (61 fold) compared to the isotype control. NR.N6.Ab1 does not bind to CLDN4-CHO-K1 cells and MCF7 cells (which expressed CLDN3 and CLDN4). These results demonstrate that NR.N6.Ab1 selectively binds to Claudin-6.

The data further demonstrate that the anti-CLDN6 antibody NR.N6.Ab2 binds strongly to Claudin-6 and 9 and does not bind to Claudin-3 or Claudin-4.

The binding specificity of anti-Claudin-6 antibodies NR.N6.Ab1 and NR.N6.Ab2 and the relevant positive control (PC) antibodies on Claudin-6-CHO-K1 cells, Claudin-9-HEK293 cells, Claudin-3-CHO-K1 cells, Claudin-4-CHO-K1 cells, Claudin-6 endogenously expressing cell lines NEC8 and OV90, and Claudin-3 and 4 endogenously expressing cell line MCF7 are summarized in Table 6 below. Binding selectivity was determined by comparing MFI of the anti-CLDN antibodies to MFI of the isotype control antibodies. Note: [-] denotes no binding observed compared to the isotype controls; n/d indicates no data; and entry marked with an asterick (*) provides data that is not showed in the figures.

TABLE 6
Summary of anti-CLDN6 binding profiles

| | | | | | | | | | MCF7 |
|---|---|---|---|---|---|---|---|---|---|
| | | | | | | | | | (CLDN3+ |
| | CLDN6- | NEC8 | OV90 | CLDN9- | | | CLDN3- | CLDN4- | and |
| Antibody | CHO | (CLDN6+) | (CLDN6+) | Hek293 | CHO | HEK293 | CHO | CHO | CLDN4+) |
| CLDN3 PC | — | — | — | — | n/d | n/d | 61-fold | — | 20-fold |
| CLDN4 PC | — | — | — | — | n/d | n/d | 9.7-fold* | 33-fold | 15-fold |
| NR.N6.Ab1 | 28-fold | 27-fold | 19-fold | 16-fold | — | — | 12-fold | — | — |
| NR.N6.Ab2 | 24-fold | 25-fold | 17-fold | 53-fold | — | — | — | — | — |
| NR.N6.PC1 | 25-fold | 19-fold | 20-fold | 15-fold | — | — | — | — | — |
| NR.N6.PC2 | 25-fold | 20-fold | 15-fold | 32-fold | — | — | — | 4.5-fold | — |

NR.N6.Ab1 and NR.N6.Ab2 were evaluated for their binding affinity to Claudin-6 overexpressing cell lines by FACS. Briefly, NR.N6.Ab1 and NR.N6.Ab2 along with NR.N6.PC1 and NR.N6.PC2 were serially diluted and tested by FACS as described above for binding to HEk293 overexpressing Claudin-6, HEK293 overexpressing Claudin-9, and CHO over expressing Claudin-6.

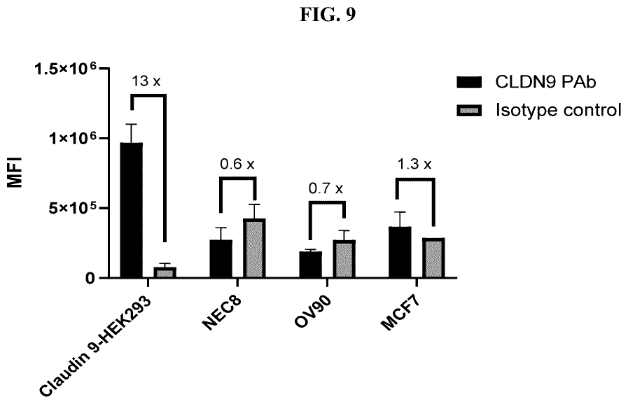

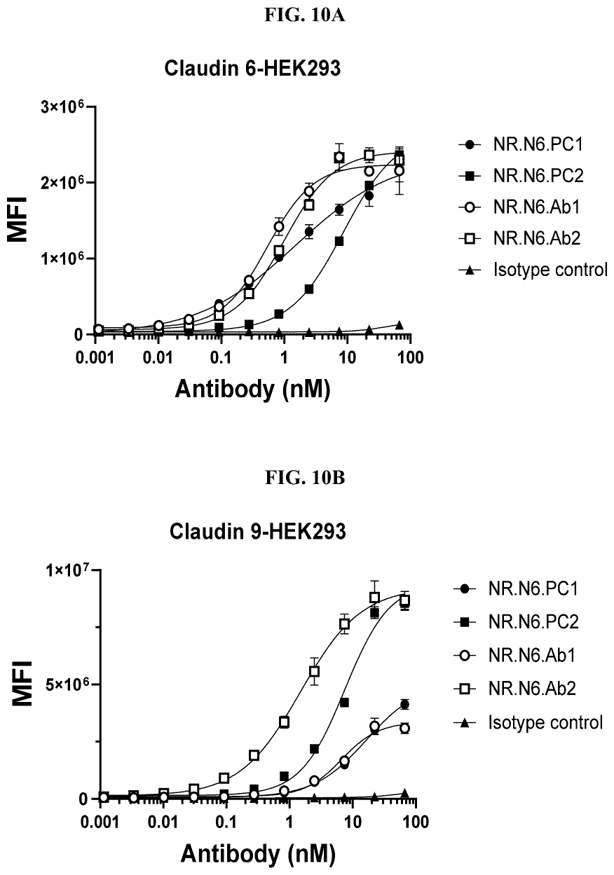

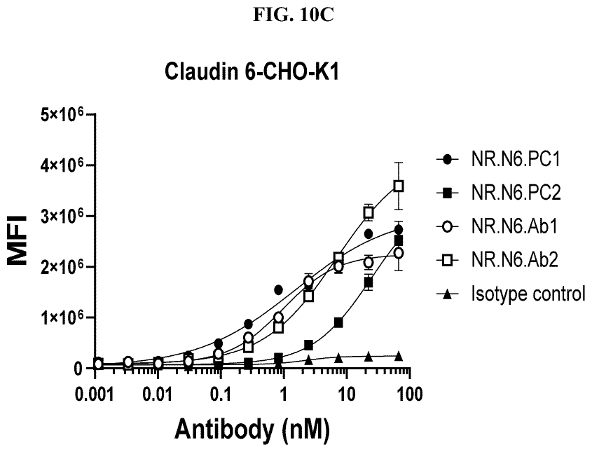

FIGS. 10A, 10B and 10C showed NR.N6.Ab1 and NR.N6.Ab2 bound to these tested cell lines in dose-dependent manner. The EC50 values are summarized in Table 7 below.

Results from the FACS experiments indicated that NR.N6.Ab1 bound to Claudin-6 with high affinity (EC50 0.55 nM on Claudin 6-HEK293 cell and 0.97 nM on Claudin-6-CHO cells). It bound to Claudin-9-HEK293 cells with lower affinity (6.72 nM) compared to binding to Claudin-6-HEK293 cells. NR.N6.Ab1 binding pattens on these tested cell lines are similar to the positive control NR.N6.PC1.

The anti-Claudin-6 antibody NR.N6.Ab2 bound to Claudin-6 and Claudin-9 with similar affinity, EC50 of 1.00 nM on Claudin-6-HEK293 cells and 1.49 nM on Claudin-9-HEK293 cells, respectively. It bound to Claudin-6-CHO cells with low nM of EC50 (6.88 nM). The binding patterns and affinity on these cell lines are similar to the positive control NR.N6.Ab2.

TABLE 7
Summary of NR.N6.Ab1 and NR.N6.Ab2 binding (EC₅₀ values)
on Claudin-6 and Claudin-9 expressing cell lines

| | Claudin-6- | Claudin-9- | Claudin-6- |
|---|---|---|---|
| | HEK293 cells, | HEK293 cells, | CHO cells |
| | EC₅₀ | EC₅₀ | EC₅₀ |
| Antibody | (nM) | (nM) | (nM) |
| NR.N6.Ab1 | 0.55 | 6.72 | 0.97 |
| NR.N6.Ab2 | 1.00 | 1.49 | 6.88 |
| NR.N6.PC1 | 1.51 | 16.36 | 1.52 |
| NR.N6.PC2 | 8.73 | 7.57 | 24.51 |

### Example 3: Antibody-Dependent Cellular Cytotoxicity (ADCC) in Tumor Cells Endogenously Expressing Claudin-6

The ADCC activity of the anti-CLDN6 antibodies NR.N6.Ab1 and NR.N6.Ab2 bound to various human Claudin-6 positive cells was measured by a bioluminescence assay. Briefly, anti-CLDN6 antibodies were serially diluted in assay buffer containing RPMI+4% low IgG FBS and added to a mixture of individual target cell line and ADCC effector cells. The ADCC effector cells are Jurkat cells expressing CD16A which were activated upon recognition of the Fc portion of the bound Claudin-6 antibodies. The activation of the effector cells was detected using a Promega bioluminescence assay following the manufacturer's instruction (Promega, cat #E6130).

ADCC activity was measured on NEC8 cell line, which has endogenous levels of Claudin-6 expression, and lacks other Claudin family members such as Claudins 3, 4, and 9.

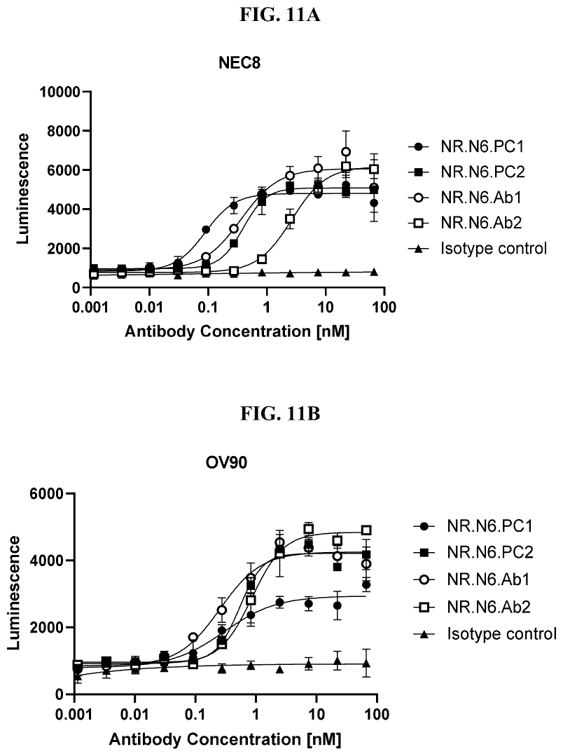

As shown in FIG. 11A and Table 8, NR.N6.Ab1 and NR.N6.Ab2 both enhanced ADCC activity on NEC8 cells. NR.N6.Ab1 and NR.N6.Ab2 exhibited EC₅₀ values of 0.64 nM and 2.77 nM, respectively.

TABLE 8
ADCC activity of anti-CLDN6 antibodies in NEC8 cell line

EC₅₀
[nM]

| | | Std. |
|---|---|---|
| mAb | Mean | Dev. |
| NR.N6.PC1 | 0.17 | 0.08 |
| NR.N6.PC2 | 0.57 | 0.18 |
| NR.N6.Ab1 | 0.64 | 0.27 |
| NR.N6.Ab2 | 2.77 | 0.12 |

As described in the above example, Claudin-6 has a lower expression in OV90 cells. As shown in FIG. 11B and table 9, ADCC activity was also observed for NR.N6.Ab1 and NR.N6.Ab2 on OV90 cells. NR.N6.Ab1 and NR.N6.Ab2 exhibited EC₅₀ levels of 0.3 nM and 0.75 nM, respectively, which is comparable to the EC₅₀ values of NR.N6.PC1 and NR.N6.PC2, 0.28 nM and 0.52 nM.

TABLE 9
ADCC activity of anti-human Claudin-6
antibodies in OV90 cell line

EC₅₀
[nM]

| | | Std. |
|---|---|---|
| mAb | Mean | Dev. |
| NR.N6.PC1 | 0.28 | 0.02 |
| NR.N6.PC2 | 0.52 | 0.02 |
| NR.N6.Ab1 | 0.30 | 0.03 |
| NR.N6.Ab2 | 0.75 | 0.11 |

### Example 4: Antibody-Mediated Endocytosis (ADC)

Endocytosis of the disclosed Claudin-6-specific antibodies bound to Claudin-6 positive cells was measured by a cytotoxicity-based endocytosis assay that used the co-internalization of the target bound antibody together with an anti-Human IgG Fc-MMAF Antibody.

NEC8, OV90, and HEK-Claudin-6 cells were cultured in growth media (RPMI1640+10% FBS, MCDB with Media 199 (1:1)+15% FBS, DMEM+10% FBS with 0.5 ug/mL Puromycin, respectively). The cells were harvested and resuspended in their respective growth media and plated into the assay plate. The cells were incubated overnight at 37° C. Anti-CLDN6 antibodies were pre-incubated with MMAF-conjugated Fab anti-hFc fragment (Moradec, Cat #AH-202AF-50), then added to cell plates and incubated for additional 96 h. CellTiter-Glo (Promega, Cat #G7570) was added to assess cell viability in each well. The signal was quantified using Neo2 plate reader (BioTek).

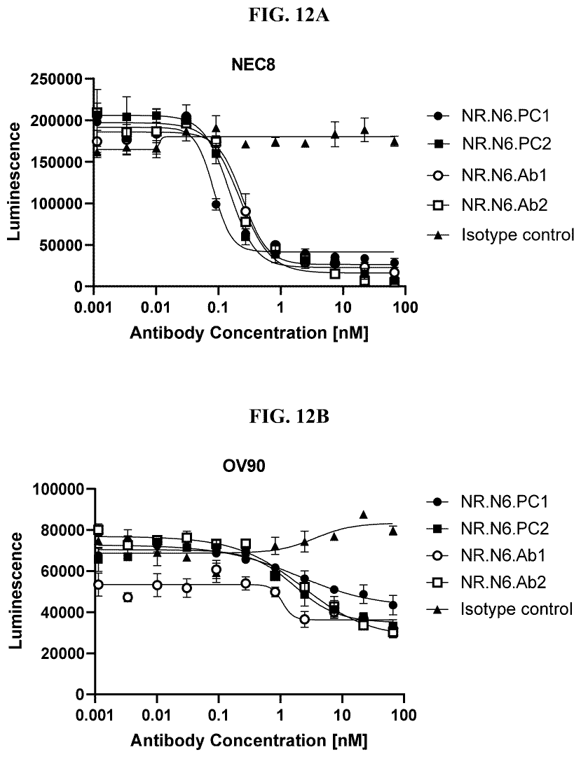

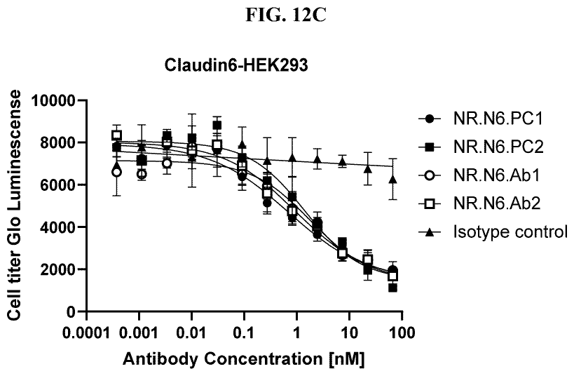

As shown in Table 10 and FIG. 12A NR.N6.Ab1 and NR.N6.Ab2 and the in-house positive control antibodies induced endocytosis-mediated cell cytotoxicity in NEC8 cells endogenously expressing Claudin-6 with EC₅₀ values ranging from 0.1 to 0.2 nM.

TABLE 10
EC₅₀ values of antibody dependent endocytosis of on NEC8 cells

EC₅₀
[nM]

| | | St. |
|---|---|---|
| mAb | Mean | Dev. |
| NR.N6.PC1 | 0.05 | 0.03 |
| NR.N6.PC2 | 0.14 | 0.04 |
| NR.N6.Ab1 | 0.20 | 0.11 |
| NR.N6.Ab2 | 0.10 | 0.07 |

OV90 is a cell line that endogenously expresses Claudin-6, albeit at a lower expression level than on NEC8 cells. As shown in Table 11 and FIG. 12B, endocytosis-mediated cell cytotoxicity was similar across all test antibodies. NR.N6.Ab1 and NR.N6.Ab2 exhibit EC₅₀ values of 1.08 and 2.32 nM, respectively.

TABLE 11
EC₅₀ values of antibody dependent endocytosis on OV90 cells

| | EC₅₀ |
|---|---|
| mAb | [nM] |
| NR.N6.PC1 | 1.96 |
| NR.N6.PC2 | 1.71 |
| NR.N6.Ab1 | 1.08 |
| NR.N6.Ab2 | 2.32 |

A HEK-293 cell line generated to recombinantly express Claudin-6 was also used to test the endocytosis-mediated cell cytotoxicity. As shown in FIG. 12C and Table 12 NR.N6.Ab1 and NR.N6.Ab2 and the in-house positive control antibodies all direct endocytosis-mediated cell cytotoxicity. The EC₅₀ values ranged from 1.73 to 2.19 nM, respectively.

TABLE 12
EC₅₀ values of antibody dependent
endocytosis on CLDN6-HEK293 cells

EC₅₀
[nM]

| | | Std. |
|---|---|---|
| Antibody | Mean | Dev. |
| NR.N6.PC1 | 1.05 | 0.35 |
| NR.N6.PC2 | 2.27 | 0.83 |
| NR.N6.Ab1 | 2.19 | 0.36 |
| NR.N6.Ab2 | 1.73 | 0.79 |

### Example 5: Binding Specificity of Anti-CLDN6 Antibodies

Anti-Claudin-6 antibodies, NR.N6.Ab3, NR.N6.Ab4, NR.N6.Ab5 and NR.N6.Ab6 were assessed for binding to Claudin-6-HEK293 (GenScript, Item #U3288DL180_3) and Claudin9-HEK293 cells (GenScript, Item #U3288DL180_4) and negative HEK293 (ATCC, CRL1573) cells by FACS. Briefly, Claudin-6-HEK293, Claudin-9-HEK293 and HEK293 cells were incubated with the Claudin-6 antibodies, NR.N6.Ab3, NR.N6.Ab4 and NR.N6.Ab5 recombinant antibodies, and NR.N6.Ab6 (purified from hybridoma supernatant) for 2 hours at 4° C. Cells were fixed with 2% PFA (Alfa Aesar, cat #: J61899) for 15 minutes at 4° C. and then washed. As used herein the term “recombinant antibody” means an engineered to comprise a human IgG1 constant region. Specific antibody binding was detected with secondary antibody goat-anti-human IgG conjugated with Alexa Fluor 647 (ThermoFisher Scientific, cat #: A21445) for the recombinant antibodies (NR.N6.Ab3 to Ab5) and goat-anti-mouse IgG conjugate with Alexa Fluor 647 (ThermoFisher Scientific, cat #: A21235) for the purified antibody NR.N6.Ab6, after one-hour incubation at 4° C. Flow cytometric analyses were performed on a flow cytometry instrument (Intellicyte, IQue plus, Sartorius).

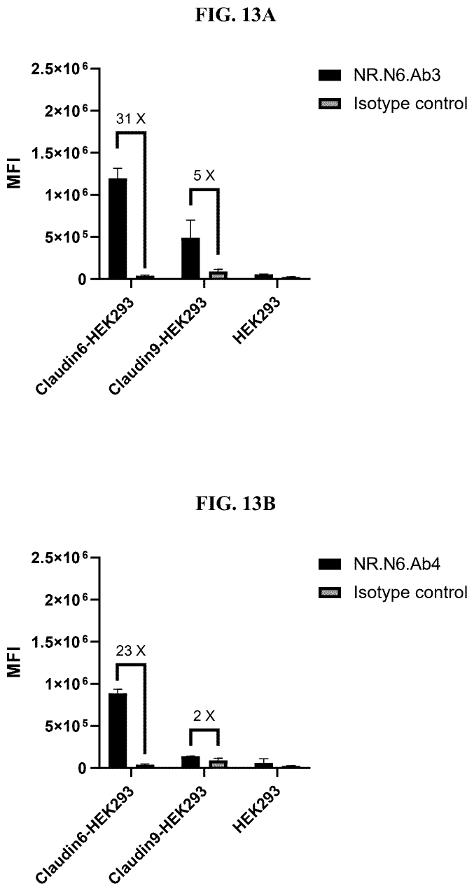

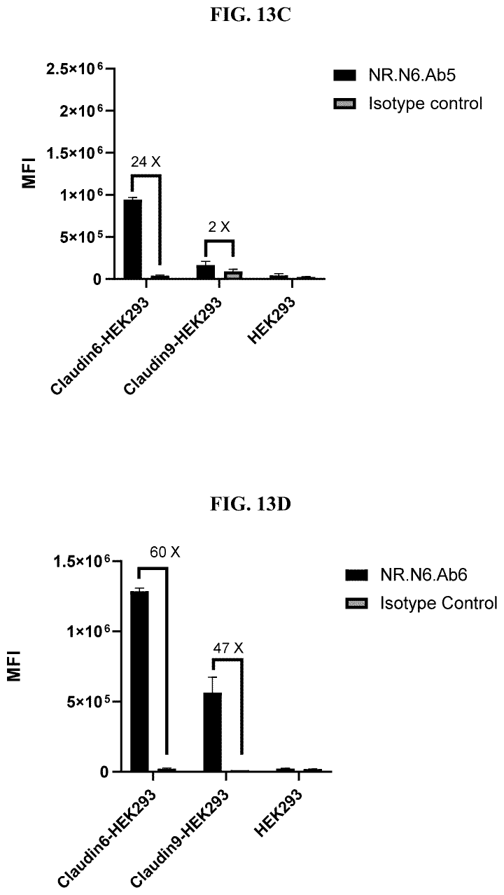

FIGS. 13A, 13B, 13C and 13D showed that the disclosed anti-Claudin-6 antibodies, NR.N6.Ab3, NR.N6.Ab4, NR.N6.Ab5 and NR.N6.Ab6, bound to Claudin-6-HEK293 transfected cells with 31-fold, 23-fold, 24-fold and 60-fold MFI, respectively, compared to the isotype control antibody staining at 5 μg/ml (NR.N6.Ab3-5) and 10 μg/ml (NR.N6.Ab6). These antibodies, NR.N6.Ab3, NR.N6.Ab4,NR.N6.Ab5 and NR.N6.Ab6, bound to Claudin-9-HEK293 transfected cells with 5-fold, 2-fold, 2-fold and 47-fold MFI, respectively, compared to the isotype control antibody staining at 5 g/ml (NR.N6.Ab3-5) and 10 μg/ml (NR.N6.Ab6).

The results indicated that the anti-CLDN6 antibodies bind preferentially to Claudin-6 over Claudin-9.

To determine whether the anti-Claudin-6 antibodies NR.N6.Ab3, NR.N6.Ab4 NR.N6.Ab5 and NR.N6.Ab6 and NR.N6.Ab1 can bind to CLDN6 expressed on NEC8 endogenously and CLDN6 gene knockout of NEC8 (NEC8 Claudin-6 KO) cells, these antibodies, along with an isotype control antibody were assessed by FACS. Briefly, NEC8 and NEC8 Claudin-6 KO cells were incubated with the Claudin-6 antibodies NR.N6.Ab3, NR.N6.Ab4, NR.N6.Ab5 (recombinant), NR.N6.Ab6 (purified from hybridoma supernatant) and NR.N6.Ab1 (recombinant) for 2 hours at 4° C. Cells were fixed with 2% PFA (Alfa Aesar, catalog number: J61899) for 15 minutes at 4° C. and then washed. Specific antibody binding was detected with secondary antibody goat-anti-human IgG conjugated with Alexa Fluor 647 (Thermo Fisher Scientific, catalog number: A21445) for the recombinant antibodies (NR.N6.Ab1, and NR.N6.Ab3 to Ab5) and anti-mouse IgG conjugate with Alexa Fluor647 (Thermo Fisher Scientific, catalog number: A21235) for NR.N6.Ab6 (purified from hybridoma supernatant) after one-hour incubation at 4° C. Flow cytometric analyses were performed on a flow cytometry instrument (Intellicyte, IQue plus, Sartorius).

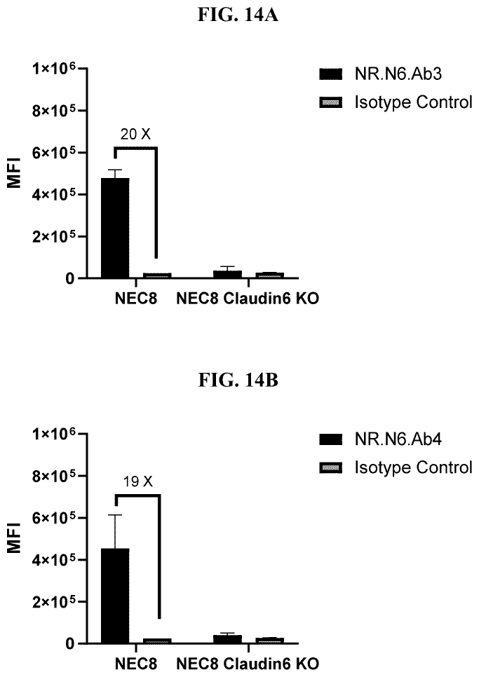

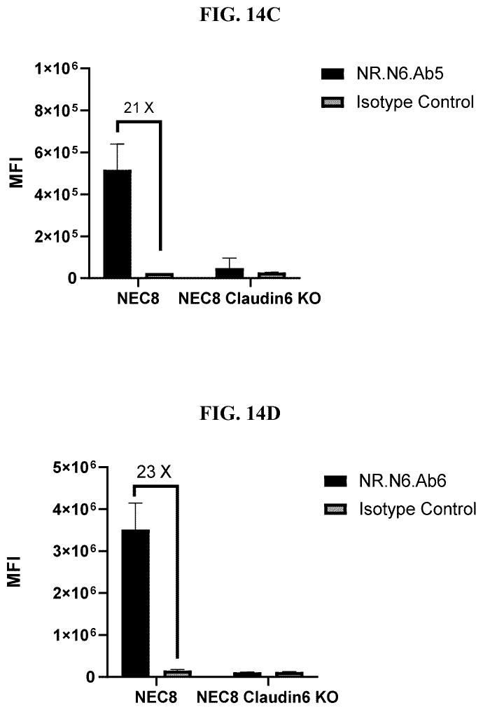

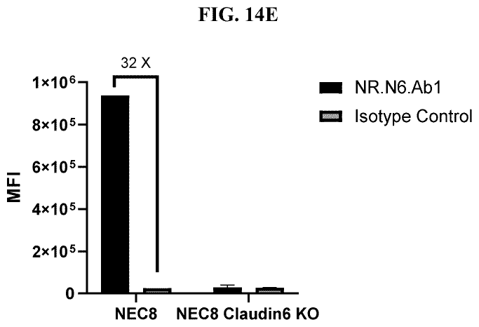

FIGS. 14A, 14B, 14C, 14D and 14E showed that the disclosed anti-CLDN6 antibodies, NR.N6.Ab3, NR.N6.Ab4, NR.N6.Ab5, NR.N6.Ab6 and NR.N6.Ab1, bound to NEC8 cells endogenously expressing Claudin-6 with 20-fold, 19-fold, 21-fold, 23-fold and 32-fold MFI, respectively. They did not bind to NEC8 CLDN6 gene knockout cells compared to the isotype control antibody staining at 5 ng/ml (for NR.N6.A3 to Ab5) or at 10 μg/ml (for NR.N6.Ab6). NR.N6.Ab1 showed similar binding pattern (14E) as previously reported in Example 2.

To further evaluate the binding characteristics of the NR.N6.Ab3, NR.N6.Ab4, NR.N6.Ab5 (recombinant antibodies), and NR.N6.Ab6 (purified from hybridoma supernatant) were tested for binding to Claudin-6-CHO-K1, Claudin-3-CHO-K1 and Claudin-4-CHO-K1 cells by FACS as described in Example 2. The anti-Claudin antibodies recognized native epitopes (mouse anti-Claudin3 IgG2a (R&D, MAB4620), mouse anti-Claudin4 IgG2a (R&D, MAB4219)). US patent 2016/0222125 A1 antibodies were used as Claudin3 and Claudin4 positive controls to confirm the expression levels of Claudin3 and Claudin4 on the CHO-K1 cells.

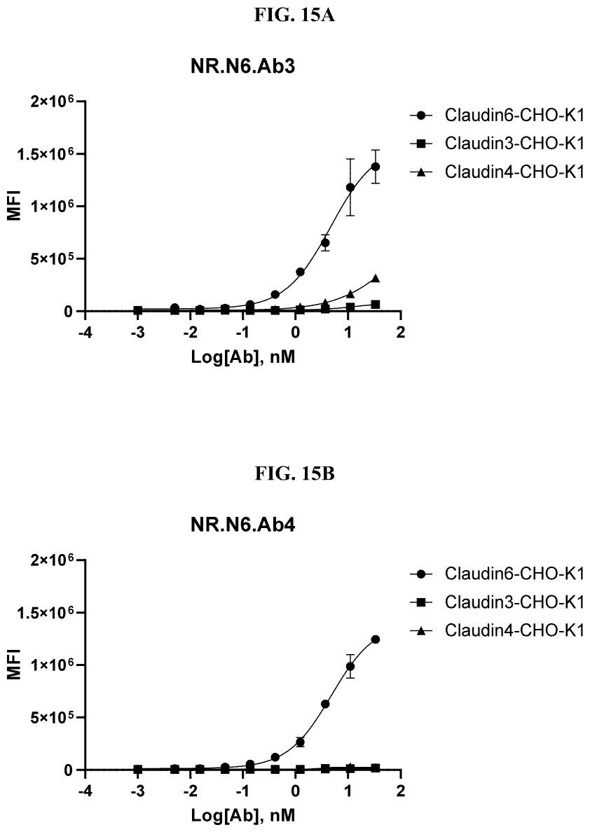

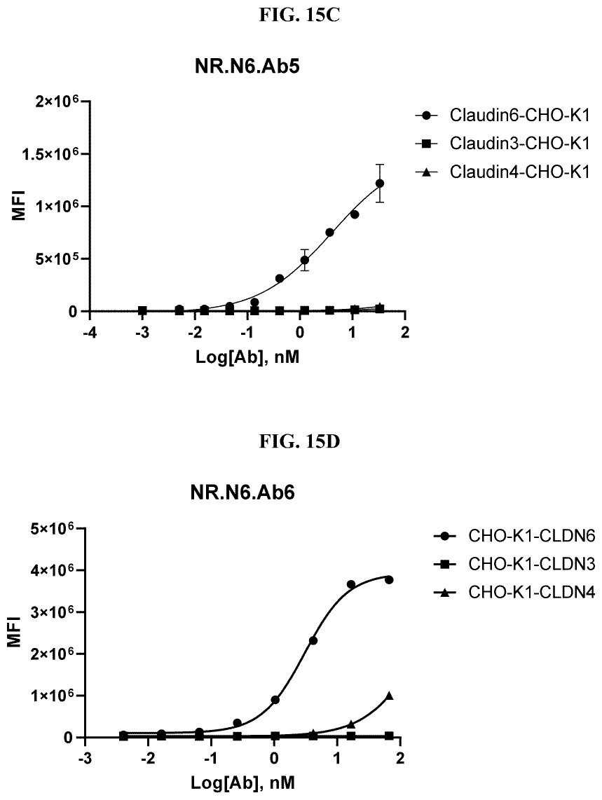

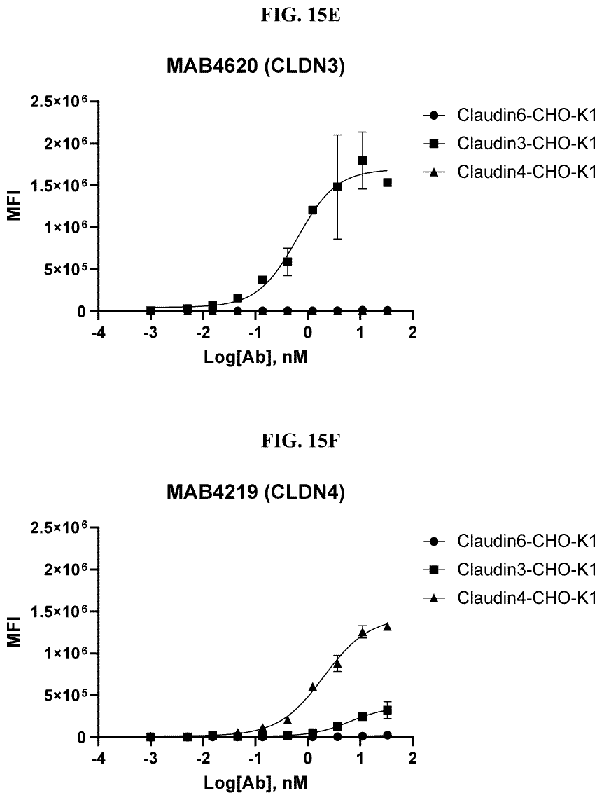

FIGS. 15A, 15B, 15C and 15D establish that, NR.N6.Ab3, NR.N6.Ab4, NR.N6.Ab5 and NR.N6.Ab6, bind to Claudin-6-CHO-K1 cells in a dose-dependent manner while they did not bind to Claudin-3-CHO-K1 and Claudin-4-CHO-K1 cells. FIGS. 15E and 15F show the positive controls antibodies, MBA4620 (anti-Claudin 3) and MBA4219 (anti-Claudin 4), bind to Claudin3-CHO-K1 and Claudin4-CHO-K1 in a dose-dependent manner, respectively.

The data further demonstrate that NR.N6.Ab3, NR.N6.Ab4, NR.N6.Ab5 and NR.N6.Ab6, bind strongly to Claudin-6 and either do not bind to Claudin-3 or Claudin-4 or are characterized by limited binding activity for Claudin-4 (NR.N6.Ab3, NR.N6.Ab6).

Table 13 summarizes the binding profiles of NR.N6.Ab3, NR.N6.Ab4, NR.N6.Ab5 and NR.N6.Ab6, and the relevant positive control antibodies on Claudin-6-HEK293 cells, Claudin-6-CHO-K1 cells, Claudin-9-HEK293 cells, Claudin-3-CHO-K1 cells, Claudin-4-CHO-K1 cells, Claudin-6 endogenously expressing cell lines of NEC8 and Claudin-6 gene knockout of NEC8 cells. Binding selectivity was determined by comparing MFI of the anti CLDN6 antibodies to MFI of the isotype control antibodies. Note: [-] denotes no binding observed compared to the isotype controls and * denotes CLDN6 gene knock-out cells.

TABLE 13
Summary of anti-CLDN6 antibody binding profiles

| | NEC8 | NEC8 | CLDN6- | CLDN9- | CHO- | | CLDN3- | CLDN4- |
|---|---|---|---|---|---|---|---|---|
| Ab | (CLDN6+) | (CLDN6−)* | HEK293 | HEK293 | K | HEK293 | CHO-K1 | CHO-K1 |
| NR.N6.Ab3 | 20- fold | — | 31-fold | 5-fold | — | — | — | —/weak+ |
| NR.N6.Ab4 | 19- fold | — | 23-fold | 2-fold | — | — | — | — |
| NR.N6.Ab5 | 21- fold | — | 24-fold | 2-fold | — | — | — | — |
| NR.N6.Ab6 | 23- fold | — | 60-fold | 47-fold | — | — | — | —/weak+ |

The anti-CLDN6 antibodies were also evaluated for their binding affinity to Claudin-6 overexpressing cell lines by FACS. Briefly, NR.N6.Ab3, NR.N6.Ab4 and NR.N6.Ab5 (recombinant antibodies), and NR.N6.Ab6 (purified from hybridoma supernatant) were serially diluted and tested by FACS as described above for binding to HEK293 overexpressing Claudin-6, HEK293 overexpressing Claudin-9, CHO overexpressing Claudin-6, and to NEC8 endogenously expressing Claudin-6.

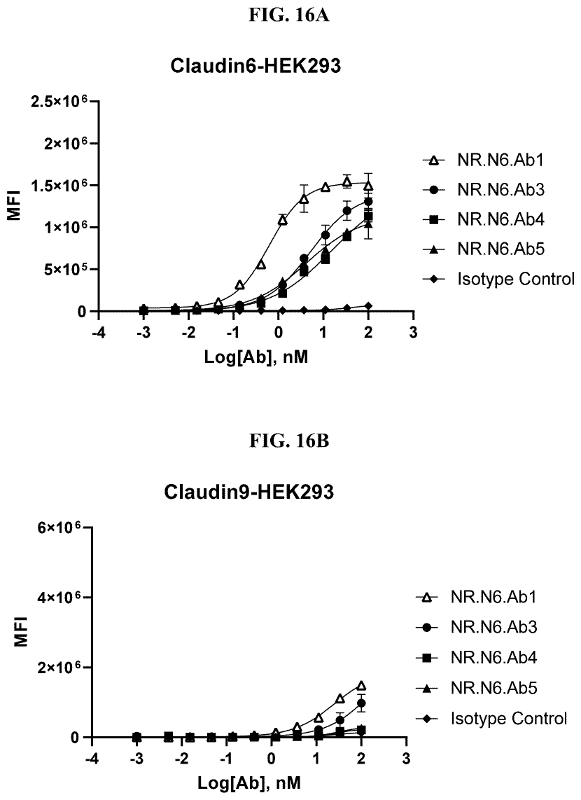

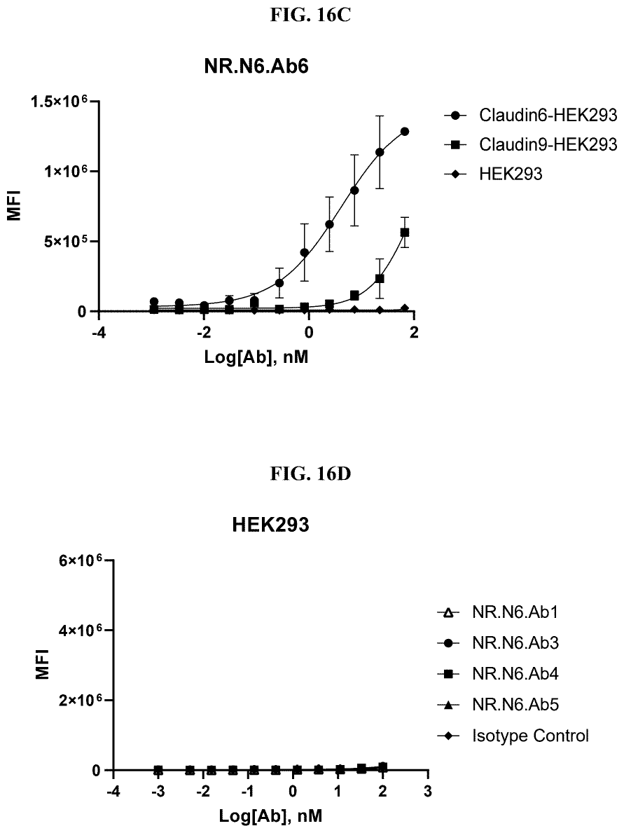

FIGS. 16A and 16C show that NR.N6.Ab3, NR.N6.Ab4, NR.N6.Ab5 and NR.N6.Ab6 bind to Claudin-6-HEK293 cells in a dose-dependent manner while they bind only minimally to Claudin-9-HEK293 cells (FIG. 16B) or weakly (16C). FIGS. 16A and 16B summarizes the binding activity of NR.N6.Ab1.

No significant binding was detected on the parental HEK293 cells (FIG. 16D). Mouse IgG isotype control was included for NR.N6.Ab6 with no binding to any of the cell lines (data not shown).

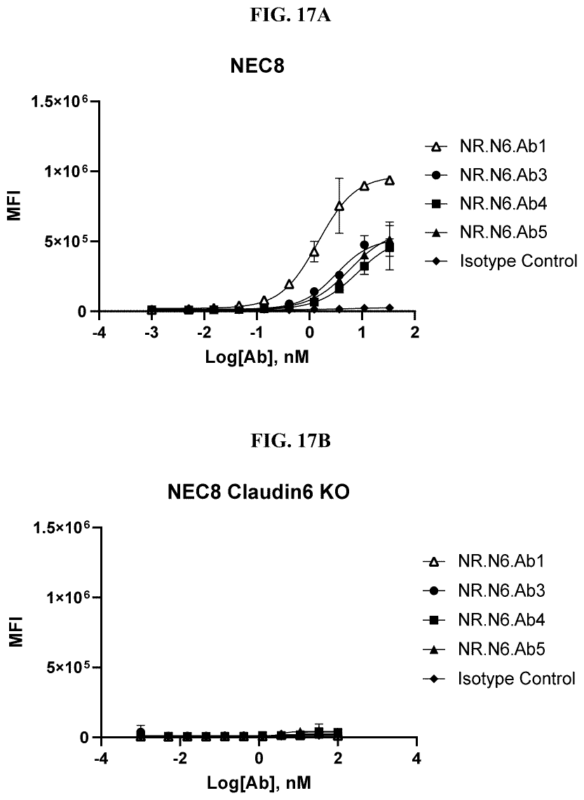

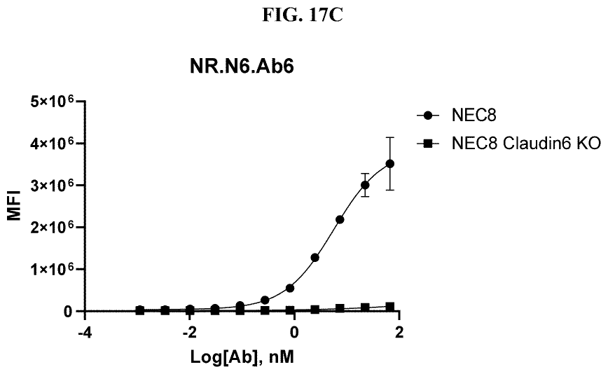

FIG. 17A establishes that the binding NR.N6.Ab3, NR.N6.Ab4 and NR.N6.Ab5, to NEC8 cells endogenously expressing Claudin-6 in a dose-dependent manner and a complete lack of binding to NEC8 Claudin-6 knockout cells (FIG. 17B). FIG. 17C showed NR.N6.Ab6 bound to NEC8 cells and did not bind to NEC8 Claudin-6 knockout cells.

These results indicated that the disclosed antibodies, NR.N6.Ab3, 4, 5 and 6, bind to Claudin-6 specifically and preferentially.

The EC₅₀ values (extracted from duplicate experiments) of these anti-Claudin-6 antibodies binding to Claudin-6-HEK293, Claudin-6-CHO-K1 and NEC8 are shown in Table 14 below Note: #denotes human cell line endogenously expressing CLDN6.

TABLE 14
Summary of EC₅₀ ValuesClaudin-6 by FACS

| | Claudin-6- | Claudin-6- | |
|---|---|---|---|
| | HEK293 cells, | CHO-K1 cells | NEC8# |
| | EC₅₀ | EC₅₀ | EC₅₀ |
| Antibody | (nM) | (nM) | (nM) |
| NR.N6.Ab3 | 5.17 | 4.69 | 3.27 |
| NR.N6.Ab4 | 15.67 | 4.79 | 8.34 |
| NR.N6.Ab5 | 4.13 | 3.99 | 5.92 |
| NR.N6.Ab6 | 4.11 | 3.06 | 5.63 |

The data establishes that NR.N6.Ab3, NR.N6.Ab4, NR.N6.Ab5 and NR.N6.Ab6, bind to Claudin-6 expressing cell lines with EC50 values ranging from: 4.11 nM to 15.67 nM on Claudin-6-HEK293; 3.06 nM to 4.79 nM on Claudin-6-CHO cells; and 3.27 nM to 8.34 nM on NEC8 cells.

Sequenced VH and VL were routinely examined for obvious liabilities including N-linked glycosylation site and additional/missing Cysteine residues. As an example, the VH of NR.N6.Ab1 (SEQ ID NO: 1) contained a N-linked glycosylation site in the FR3 (N73 counting from the N-terminus). The N-linked glycosylated was removed by mutating the Asn to Asp guided by the homologous germline sequences, resulting in SEQ ID NO: 23.

NR.N6.Ab1 variant N73D along with NR.N6.Ab1 and an isotype control antibody were assessed for their binding specificity and affinity to Claudin-6-HEK293, Claudin-9-HEK293, NEC8 cells endogenously expressing Claudin-6 and NEC8 Claudin-6 knockout cells by FACS as described above.

The EC₅₀ values of these anti-Claudin-6 antibodies, NR.N6.Ab1 N73D and NR.N6.Ab1, binding to the Claudin-6-HEK293 cells and NEC8 cells endogenously expressing Claudin-6 are extracted from duplicate experiments are summarized in Table 15.

TABLE 15
Summary of NR.N6.Ab1 and NR.N6.Ab1 N73D binding
to CLDN6 expressing cell lines by FACS

| | Claudin-6- | |
|---|---|---|
| | HEK293 cells, | NEC8# |
| | EC₅₀ | EC₅₀ |
| Antibody | (nM) | (nM) |
| NR.N6.Ab1 | 0.63 | 1.39 |
| NR.N6.Ab1 N73D | 0.42 | 0.74 |
| LALA | | |

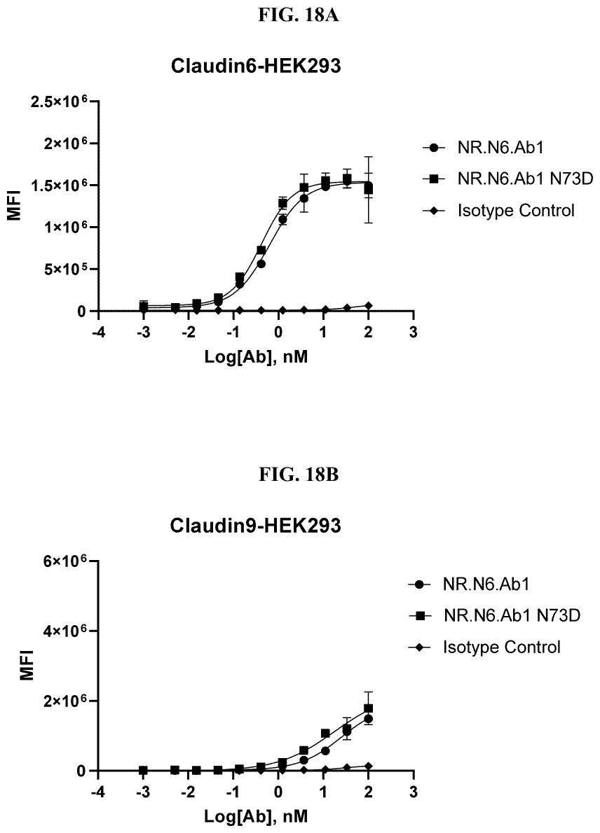

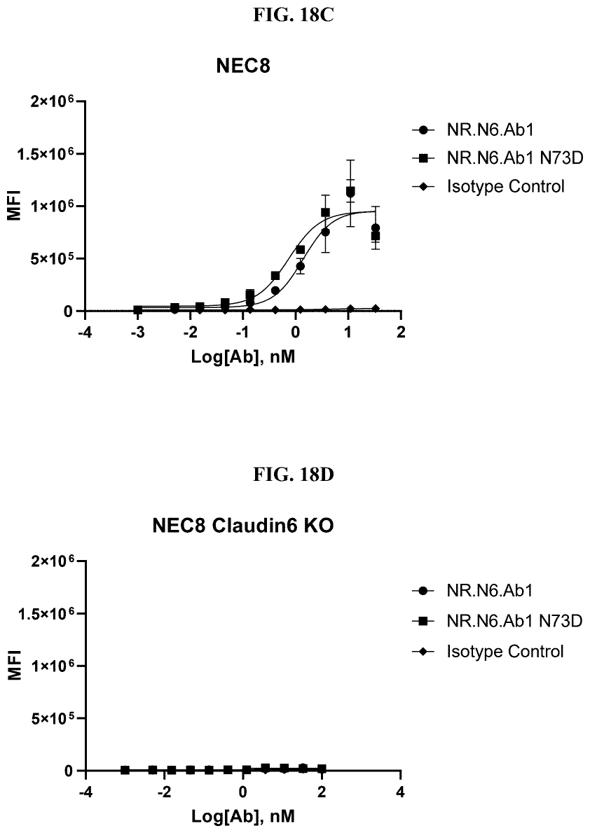

FIGS. 18A, 18B and 18C showed that NR.N6.Ab1 and NR.N6.Ab1 variant N73D bound to Claudin-6-HEK293 cells (18A) and NEC8 cells (18C) in a dose-dependent manner, and that the NR.N6.Ab1 variant N73D exhibits a similar binding profile to the parental NR.N6.Ab1. They bound to Claudin-9-HEK293 cells with much lower activities (18B) and did not bind to NEC8 Claudin-6 knockout cells (18D).

### Example 6: Antibody-Dependent Cellular Cytotoxicity (ADCC) in Tumor Cells Endogenously Expressing Claudin-6

The ADCC activity of NR.N6.Ab3, NR.N6.Ab4 and NR.N6.Ab5 and NR.N6.Ab1 was measured by a bioluminescence assay. Briefly, anti-CLDN6 antibodies were serially diluted in assay buffer containing RPMI+4% low IgG FBS and added to a mixture of individual target cell line and ADCC effector cells. The ADCC effector cells are Jurkat cells expressing CD16A which were activated upon recognition of the Fc portion of the bound Claudin-6 antibodies. The activation of the effector cells was detected using a Promega bioluminescence assay following the manufacturer's instruction (Promega, cat #E6130).

ADCC activity was measured on NEC8, which has endogenous levels of Claudin-6 expression and lacks other Claudin family members such as Claudins 3, 4, and 9 (Sahin, U. et. al. 2016), and NEC Claudin-6 KO (NEC8 Claudin-6 knockout cell line).

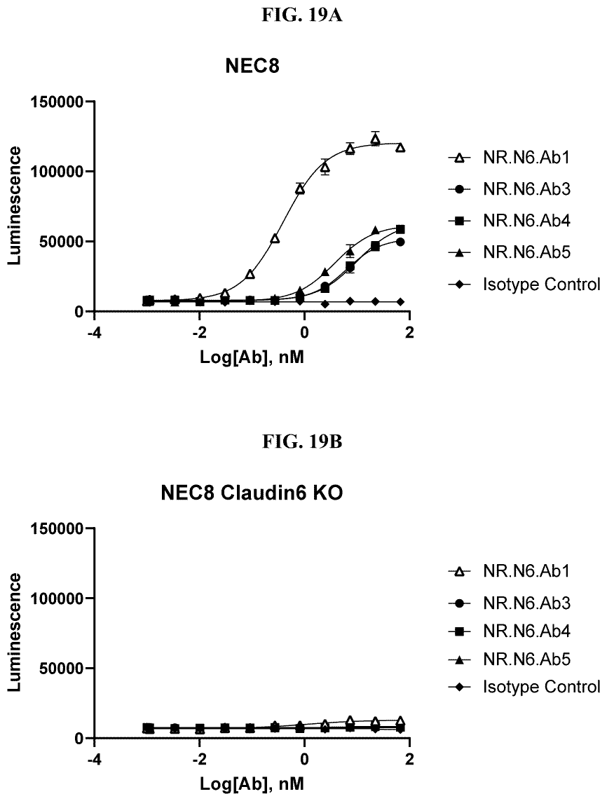

As shown in FIG. 19A and Table 16, NR.N6.Ab3, NR.N6.Ab4 and NR.N6.Ab5 induced ADCC activity on NEC8 cells with EC₅₀ values of 6.43 nM, 9.83 nM and 3.78 nM, respectively. NR.N6.Ab1 (included as a positive control) consistently exhibits ADCC activity with an EC50 value of 0.40 nM compared to the previous EC50 value of 0.64 nM (Example 3, Table 8). All the EC50 values are extracted from duplicate experiments. No ADCC activity was detected in the NEC8 Claudin-6 KO cells (19B).

TABLE 16
ADCC activity of anti-Claudin-6 Antibodies on NEC8 Cells

| | EC50 |
|---|---|
| Antibody | (nM) |
| NR.N6.Ab1 | 0.40 |
| NR.N6.Ab3 | 6.43 |
| NR.N6.Ab4 | 9.83 |
| NR.N6.Ab5 | 3.78 |

### Example 7: Internalization of Anti-CLDN6 Antibodies

Internalization of NR.N6.Ab1, NR.N6.Ab3, NR.N6.Ab4 and NR.N6.Ab5 was measured by immunofluorescence imaging assay using NEC8, or NEC8 Claudin-6 knockout cells. The cells were plated in complete media containing RPMI-1640 with 10% FBS, then incubated overnight at 37° C. The antibodies were first chemically conjugated with fluorescent dye using Alex Fluor™ 488 antibody labelling kit (ThermoFisher, A20181). Excess amount of unconjugated dye was removed using Zeba™ spin desalting columns, 40K MWCO (ThermoFisher, 87766). Cells were then incubated with 10 μg/ml of fluorescent labelled antibodies at 4° C. for 4 hours. After antibody pre-binding, the corresponding plates were incubated at 37° C. for 0, 4 and 24 hours followed by fixing cells with paraformaldehyde for 15 minutes at room temperature. The fixed cells were washed with PBS for three times followed by incubating with anti-Alexa Fluor 488 antibody (ThermoFisher, A11094) at room temperature for 1 hour to quench the extracellular cell surface signal. The fluorescent signal of internalized antibodies was assessed by imaging the cells and quantifying the fluorescence intensity using Cytation Imager (Biotek, VT).

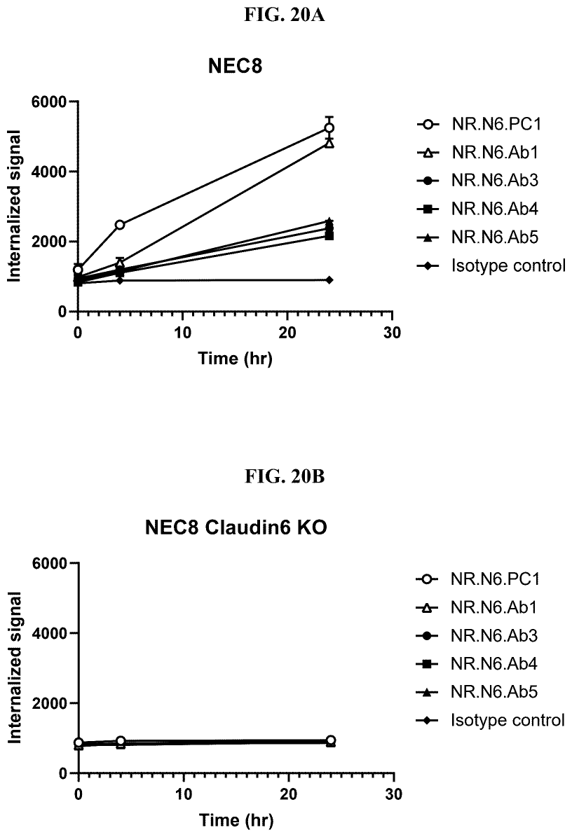

FIGS. 20A and 20B showed the disclosed antibodies internalized into NEC8 cells but not NEC8 Claudin 6 KO cells. No internalization signal was detected when human IgG1 isotype control antibody was incubated with either NEC8 cells or NEC8 Claudin-6 KO cells. This observation indicates that internalization of the disclosed antibodies was specifically through binding to Claudin-6 protein on the cell surface.

### Example 8: Antibody-Mediated Endocytosis by Anti-CLDN6 Antibodies

Endocytosis of NR.N6.Ab1, NR.N6.Ab3, NR.N6.Ab4 and NR.N6.Ab5 antibodies bound to Claudin-6 positive cells was measured by a cytotoxicity-based endocytosis assay based on the co-internalization of the target bound antibody together with an anti-human IgG Fc-MMAF Antibody.

NEC8 and NEC8 Claudin-6 knockout cells were cultured in growth media (RPMI1640+10% FBS). The cells were harvested and resuspended in the growth media and plated into the assay plate. The cells were incubated overnight at 37° C. Anti-Claudin-6 antibodies were pre-incubated with MMAF-conjugated Fab anti-hFc fragment (Moradec, Cat #AH-202AF-50), then added to cell plates and incubated for additional 72 hours. CellTiter-Glo (Promega, Cat #G7570) was added to assess cell viability in each well. The signal was quantified using Neo2 plate reader (BioTek).

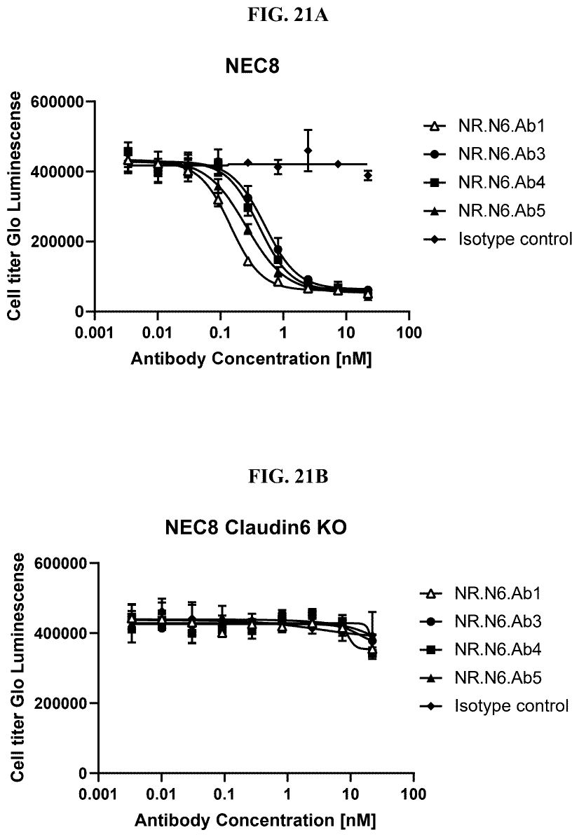

As shown in FIG. 21A and Table 17, the disclosed anti-Claudin 6 antibodies induced antibody-mediated endocytosis in the NEC8 cell line with E_{C50} values ranging from 0.14 to 0.51 nM. No endocytosis derived cell cytotoxicity was detected in the NEC8 Claudin 6 KO cell line (FIG. 21B).

TABLE 17
Summary EC₅₀ values of antibody dependent
endocytosis killing on NEC8 cells.

| | EC50 |
|---|---|
| mAb | [nM] |
| NR.N6.Ab1 | 0.14 |
| NR.N6.Ab3 | 0.51 |
| NR.N6.Ab4 | 0.41 |
| NR.N6.Ab5 | 0.26 |

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope of the disclosure otherwise claimed. No language in the specification should be construed as indicating any non-claimed element essential to the practice of the disclosure.

Groupings of alternative elements or embodiments of the disclosure disclosed herein are not to be construed as limitations. Each group member can be referred to and claimed individually or in any combination with other members of the group or other elements found herein. It is anticipated that one or more members of a group can be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this disclosure are described herein, including the best mode known to the inventors for carrying out the disclosure. Of course, variations on these described embodiments will become apparent to those of ordinary skill in the art upon reading the foregoing description. The inventor expects skilled artisans to employ such variations as appropriate, and the inventors intend for the disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

Specific embodiments disclosed herein can be further limited in the claims using “consisting of” or “consisting essentially of” language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the disclosure so claimed are inherently or expressly described and enabled herein.

It is to be understood that the embodiments of the disclosure disclosed herein are illustrative of the principles of the present disclosure. Other modifications that can be employed are within the scope of the disclosure. Thus, by way of example, but not of limitation, alternative configurations of the present disclosure can be utilized in accordance with the teachings herein. Accordingly, the present disclosure is not limited to that precisely as shown and described.

While the present disclosure has been described and illustrated herein by references to various specific materials, procedures and examples, it is understood that the disclosure is not restricted to the particular combinations of materials and procedures selected for that purpose. Numerous variations of such details can be implied as will be appreciated by those skilled in the art. It is intended that the specification and examples be considered as exemplary only, with the true scope and spirit of the disclosure being indicated by the following claims. All references, patents, and patent applications referred to in this application are herein incorporated by reference in their entirety.

What is claimed is:

1. An anti-CLDN6 antibody comprising:
(a) (i) VH: CDR1: SEQ ID NO: 5, CDR2: SEQ ID NO: 6, CDR3: SEQ ID NO: 7, VL: CDR1: SEQ ID NO: 8, CDR2: SEQ ID NO: 9, CDR3: SEQ ID NO: 10;
(b) VH: CDR1: SEQ ID NO: 11, CDR2: SEQ ID NO: 12, CDR3: SEQ ID NO: 13, VL: CDR1: SEQ ID NO: 14, CDR2: SEQ ID NO: 15, CDR3: SEQ ID NO: 16;
(c) VH: CDR1: SEQ ID NO: 32, CDR2: SEQ ID NO: 33, CDR3: SEQ ID NO: 34, VL: CDR1: SEQ ID NO: 35, CDR2: SEQ ID NO: 36, CDR3: SEQ ID NO: 37;
(d) VH: CDR1: SEQ ID NO: 38, CDR2: SEQ ID NO: 39, CDR3: SEQ ID NO: 40, VL: CDR1: SEQ ID NO: 41, CDR2: SEQ ID NO: 42, CDR3: SEQ ID NO: 43;
(e) VH: CDR1: SEQ ID NO: 38, CDR2: SEQ ID NO: 44, CDR3: SEQ ID NO: 40, VL: CDR1: SEQ ID NO: 41, CDR2: SEQ ID NO: 45, CDR3: SEQ ID NO: 43; and
(f) VH: CDR1: SEQ ID NO: 46, CDR2: SEQ ID NO: 47, CDR3: SEQ ID NO: 48, VL: CDR1: SEQ ID NO: 49, CDR2: SEQ ID NO: 50, CDR3: SEQ ID NO: 51.

2. The anti-CLDN6 antibody of claim 1, wherein the antibody comprises:
(a) a heavy chain variable region having a sequence set forth in SEQ ID NO: 1 and a light chain variable region having a sequence set forth in SEQ ID NO: 2;
(b) a heavy chain variable region having a sequence set forth in SEQ ID NO: 3 and a light chain variable region having a sequence set forth in SEQ ID NO: 4
(c) a heavy chain variable region having a sequence set forth in SEQ ID NO: 23 and a light chain variable region having a sequence set forth in SEQ ID NO: 2;
(d) a heavy chain variable region having a sequence set forth in SEQ ID NO: 24 and a light chain variable region having a sequence set forth in SEQ ID NO: 25;
(e) a heavy chain variable region having a sequence set forth in SEQ ID NO: 26 and a light chain variable region having a sequence set forth in SEQ ID NO: 27;
(f) a heavy chain variable region having a sequence set forth in SEQ ID NO: 28 and a light chain variable region having a sequence set forth in SEQ ID NO: 29; or
(g) a heavy chain variable region having a sequence set forth in SEQ ID NO: 30 and a light chain variable region having a sequence set forth in SEQ ID NO: 31.

3. The anti-CLDN6 antibody of claim 1, wherein the antibody is a human antibody.

4. The anti-CLDN6 antibody of claim 1, wherein the antibody is a chimeric antibody.

5. The anti-CLDN6 antibody according to anyone of claims 1 to 4, wherein the antibody is conjugated to a cytotoxic agent.

6. The anti-CLDN6 antibody of claim 1, wherein the antibody is a full-length antibody.

7. The anti-CLDN6 antibody of claim 1, wherein the antibody is an antibody fragment.

8. The anti-CLDN6 antibody of claim 7, wherein the antibody fragment is selected from the group consisting of: Fab, Fab, F(ab)2, Fd, Fv, scFv and scFv-Fc fragment, a single-chain antibody, a minibody, and a diabody.

9. A pharmaceutical composition comprising as an active ingredient, at least one antibody according to anyone of claims 1 to 4 and a pharmaceutically acceptable carrier.

10. A pharmaceutical composition comprising as an active ingredient, an antibody according to anyone of claim 5 and a pharmaceutically acceptable carrier.

11. The pharmaceutical composition according to any one of claim 9 or 10 for use in treating cancer.

12. A method of treating cancer comprising administering to a subject in need thereof, a pharmaceutical composition according to claim 9 or 10.

13. A method of diagnosing a cancer in a subject, the method comprising contacting a biological sample with an antibody or antibody fragment according to any one of claims 1 to 2.

14. An isolated polynucleotide comprising a sequence encoding an anti-CLDN6 antibody according to claim 1.

15. An isolated polynucleotide according to claim 14 encoding a sequence as set forth in any one of SEQ ID NOS: 1 to 4.

16. A vector comprising a polynucleotide according to claim 15.

17. A host cell comprising a polynucleotide according to claim 15, and/or a vector according to claim 16.

18. A method for the production of an anti-CLDN6 antibody according to claim 1, the method comprising culturing the host cell of claim 17.
